SEQUENCE LISTING (9 sequences)

SEQ ID NO: 1 - protein, 233 aa, Homo sapiens
MEPTAPSLTEEDLTEVKKDALENLRVYLCEKIIAERHFDHLRAKKILSREDTEEISCRTSSRKRAGKLLDYLQENPKGLDTLVESIRREKTQNFLIQKITDEVLKLRNIKLEHLKGLKCSSCEPFPDGATNNLSRSNSDESNFSEKLRASTVMYHPEGESSTTPFFSTNSSLNLPVLEVGRTENTIFSSTTLPRPGDPGAPPLPPDLQLEEEGTCANSSEMFLPLRSRTVSRQ

SEQ ID NO: 2 - DNA, 702 bp, Homo sapiens
  source 1..702: mol_type = other DNA
atggagcccaccgcaccgtccctcaccgaggaggacctcactgaagtgaagaaggacgccttagaaaatttacgtgtatacctgtgtgagaaaatcatagctgagagacattttgatcatctacgtgcaaaaaaaatactcagtagagaagacactgaagaaatttcttgtcgaacatcaagtagaaaaagggctggaaaattgttagactacttacaggaaaacccaaaaggtctggacacccttgttgaatctattcggcgagaaaaaacacagaacttcctgatacagaagattacagatgaagtgctgaaacttagaaatataaaactagaacatctgaaaggactaaaatgtagcagttgtgaaccttttccagatggagccacgaacaacctctccagatcaaattcagatgagagtaatttctctgaaaaactgagggcatccactgtcatgtaccatccagaaggagaatccagcacgacgccctttttttctactaattcttctctgaatttgcctgttctagaagtaggcagaactgaaaataccatcttctcttcaactacacttcccagacctggggacccaggggctcctcctttgccaccagatctacagttagaagaagaaggaacttgtgcaaactctagtgagatgtttcttcccttaagatcacgtactgtttcacgacaatga

SEQ ID NO: 3 - DNA, 2833 bp, Homo sapiens
  source 1..2833: mol_type = other DNA
tgcctgcgcctgagcctctacgagagggaaggaacgctgctccgagctccgcgtcgcgtcgcgtagattcgcgtcgccgtcgacctcagaggcggggccggaagcgctacggtttgacccccgagtccctctgttcccgaaggggcggccgtctttctcccgacccgctccgcctcctctccttcttccccattacccggaggccgaagcccccagccagggcggggcggcgcagcccgagctcccggacccggaagaagcgccatctcccgcctccaccatggagcccaccgcaccgtccctcaccgaggaggacctcactgaagtgaagaaggacgccttagaaaatttacgtgtatacctgtgtgagaaaatcatagctgagagacattttgatcatctacgtgcaaaaaaaatactcagtagagaagacactgaagaaatttcttgtcgaacatcaagtagaaaaagggctggaaaattgttagactacttacaggaaaacccaaaaggtctggacacccttgttgaatctattcggcgagaaaaaacacagaacttcctgatacagaagattacagatgaagtgctgaaacttagaaatataaaactagaacatctgaaaggactaaaatgtagcagttgtgaaccttttccagatggagccacgaacaacctctccagatcaaattcagatgagagtaatttctctgaaaaactgagggcatccactgtcatgtaccatccagaaggagaatccagcacgacgccctttttttctactaattcttctctgaatttgcctgttctagaagtaggcagaactgaaaataccatcttctcttcaactacacttcccagacctggggacccaggggctcctcctttgccaccagatctacagttagaagaagaaggaacttgtgcaaactctagtgagatgtttcttcccttaagatcacgtactgtttcacgacaatgacactttattgccttttaatttttaatgatgacaaaaaatgttttaaagaatatgactttttataaaatggctgtaatcatttgtttacatttgatgcatgtcttttaaaatgcaatgtaagcatactttgtaaataggatttttagaattaaaaaagcatacttctaggatagctaactgtaaatcatgttgatcatgtactttttagtaatttctttttttcctttttaaggtctttcagtacttttttaaatattttctattttaagactgattttaatagggaatatatctctatttgagaatagacccttactaggaagaacgttttttcctcagtgcatttgtgctagaaattttcaagagtctaatagtctttgccagtcattcagcagcaaattttcagcattaagctgttcctgttcagtaataaaaccggtcactgatgggaaaactgccaatatagaaaaataaaaatctcttttccactccattgtcgtataggcatgtaaacagcctctttttgatactggaggaacacttgatggagtgtgagccacctaagatctcggtttgccaaaattcatttctaattaaccttactaattatactactttgttaggattttcacattcttggcttaatcattttcattcctaaagaaaaatatcttggcctaaacctcagttattacatgtaatttgatgaggtattttttccttttttcttttttttttttttttttttttgagacagtcttgctctatcgcccaggctggagtgcagtggcgcattctaggctcactgcaacttctgcctcccatgcttacgtgatcctctcacctcagcctctcaagtaatatagctgagactacaagtgtgtgccaccatgcctcactaatttttgtattatttttgtagagacggtgttttgccatgttggccaggctggtcttgaactcctggactcaagcaacctacccagcgtggcctcccaaagtgctgggattacagacacgagccacctcacctagcctgatgagatttttaaaaaatattttctctgtacttttcattctcttttaatgaggaccaatgtacagttgaaataactggaacaaattatttttggtgtgtgtgacaattctgtttttaatgctatttgaacaagtgggccattagccagatttgtctttttgttgtaaaacaaaatttgactaattttacatgtttataaatcttatgctctcactgtttgtttttatttaaattacaattttatctgtttcctgacattgtctcctatatatttctattattaattgcaaaaacatagaaatggaaattttgctatcaacaataaaatttttttaaagtagtgagtgctattttggagttccaaattttcagtaggaagtatctaaaactttttttaatacgtgccattatctatagaaaacattacttcaggttgtgagattgagttgcatttctggatggactgatgaatttatccgacatgaagaagattggcatattagctttaaaaatttttaaagattggattttttttagtataagcactttctaaggattatagagaaatgtttcacctccaatgcatagcaaaaatagtggtgttagaaagaaaataggttacatttaaggaaggtgctttaaaaagcagaagcagactttaaaattaaatttgtggacacctttttaaaaattgaatcaaagattataatttagatatacaataacacctatatatagataagttttaacactgagttttctttcagactgttttctaactacatagacaataaaattaagctttgcataaa

SEQ ID NO: 4 - DNA, 21 bp, synthetic construct
  source 1..21: mol_type = other DNA
caacaagatgaagagcaccaa

SEQ ID NO: 5 - DNA, 21 bp, synthetic construct
  source 1..21: mol_type = other DNA
ggacgacaactacaacttagc

SEQ ID NO: 6 - DNA, 21 bp, synthetic construct
  source 1..21: mol_type = other DNA
tggtcaagaagctgacgattc

SEQ ID NO: 7 - DNA, 21 bp, synthetic construct
  source 1..21: mol_type = other DNA
caacaagatgaagagcaccaa

SEQ ID NO: 8 - DNA, 21 bp, synthetic construct
  source 1..21: mol_type = other DNA
ggacgacaactacaacttagc

SEQ ID NO: 9 - DNA, 21 bp, synthetic construct
  source 1..21: mol_type = other DNA
tggtcaagaagctgacgattc

(19) United States
(12) Patent Application Publication
(10) Pub. No.: US 2024/0026461 A1
(43) Pub. Date: Jan. 25, 2024

(54) BCL10 MUTATIONS AS BIOMARKERS FOR TARGETED THERAPY IN B CELL LYMPHOMAS

(71) Applicants: Ari Melnick, New York, NY (US); Min Xia, New York, NY (US); Hao Wu, Brookline, MA (US); Liron David, Allston, MA (US)

(72) Inventors: Ari Melnick, New York, NY (US); Min Xia, New York, NY (US); Hao Wu, Brookline, MA (US); Liron David, Allston, MA (US)

(21) Appl. No.: 18/328,226

(22) Filed: Jun. 2, 2023

Related U.S. Application Data
(60) Provisional application No. 63/349,459, filed on Jun. 6, 2022.

(51) Int. Cl. C12Q 1/6886

(52) U.S. Cl. CPC C12Q 1/6886; C12Q 2600/106; C12Q 2600/156

(57) ABSTRACT

Methods are described herein for identifying and/or treating lymphoma patients that have one or more genomic mutations in a coding region of at least one BCL10 allele. Such patients can be resistant to commonly used cancer treatments and can benefit instead from treatment with at least one MALT1 inhibitor.

### CROSS-REFERENCE TO RELATED APPLICATIONS

This application claims the benefit of the filing date of U.S. application No. 63/349,459, filed on Jun. 6, 2022, the disclosure of which is incorporated by reference herein.

### FEDERAL FUNDING

This invention was made with government support under CA220499 and CA249843 awarded by National Institutes of Health. The government has certain rights in the invention.

### INCORPORATION BY REFERENCE OF SEQUENCE LISTING

A Sequence Listing is provided herewith as an xml file, “2339852.xml” created on Jun. 2, 2023, and having a size of 12,694 bytes. The content of the xml file is incorporated by reference herein in its entirety.

### BACKGROUND

Activated B-cell diffuse large B-cell lymphomas (ABC DLBCLs) are among the most aggressive lymphomas. ABC DLBCLs typically include frequent mutations of immune signaling pathways that culminate in activation of the CARD11-BCL10-MALT1 (CBM) complex signal amplification complex. The CARD11/BCL10/MALT1 complex plays a role in integrating signaling pathways involved in immunity and inflammation in a broad repertoire of cell types. In B-cells and T-cells, the CBM complex is activated downstream of B-cell receptor (BCR) or T-cell receptor (TCR) signaling and serves to amplify such signals leading to powerful phenotype responses conferred by critical downstream mediates.

### SUMMARY

As described herein BCL10 gain-of-function mutations have been identified in subjects with diffuse large B-cell lymphomas (DLBCLs) and in activated B-cell-DLBCLs. Through biochemical, structural and functional dissection of these mutations, two distinct classes of BCL10 mutations were identified: missense mutations in the caspase activation and recruitment domain (CARD) and truncation mutations in the C-terminal region of BCL10. Treatment with MALT1 inhibitors of subjects having such mutations is particularly beneficial, and more effective than treatment with BTK inhibitors or commonly used chemotherapy regimens for advanced DLBCL. For example, when a sample from a subject indicates that the subject has a BCL10 mutation that subject is more effectively treated with MALT1 inhibitors, and in some cases not treated with BTK inhibitors. MALT1 inhibitors are particularly useful for treatment of subjects with C-terminal deletions or C-terminal truncations of at least one BCL10 allele. However, subjects having mutations in the BCL10 CARD domain can also benefit from treatment with MALT1 inhibitors.

A method is described herein that can involve administering at least one MALT1 inhibitor to a subject having a mutation in at least one BCL10 allele.

Also described herein are methods of identifying subjects who can benefit from treatment with at least one MALT1 inhibitor, the method involving screening the subjects to identify which subjects, if any, have at least one mutation in at least one BCL10 allele.

The BCL10 mutation(s) can be missense mutations, deletions, or insertions in the BCL10 coding region of at least one BCL10 allele. In some cases, the mutation is in a BCL10 CARD domain. In some cases, the mutation is a BCL10 C-terminal truncation or BCL10 C-terminal deletion in the BCL10 coding region. Subjects with at least mutation in a coding region of at least one BCL10 allele can be treated with one or more MALT1 inhibitors.

In some cases, the mutation is in the BCL10 CARD domain is a mutation at an amino acid position equivalent to position 58 of SEQ ID NO:1, or is a mutation that includes an amino acid position equivalent to position 58. In some cases, the C-terminal truncation or C-terminal deletion in a coding region of at least one BCL10 allele is within amino acid positions equivalent to position 135-174 of SEQ ID NO:1.

Subjects can be evaluated for treatment with one or more MALT1 inhibitors by a variety of methods such as genomic sequencing, polymerase chain reaction (PCR) analysis, restriction fragment length polymorphism (RFLP) analysis, mRNA size analysis, protein sequencing, antibody detection of BCL10 mutant proteins, use of primers/probes specific for truncation mutant BCL10 proteins, and the like.

A method is also described herein that can involve administering at least one MALT1 inhibitor to a subject having one or more genomic mutations in a coding region of at least one BCL10 allele, where the one or more genomic mutations can be within:
a. a BCL10 coding region mutation at an amino acid position equivalent to position 58 of SEQ ID NO:1;
b. a BCL10 coding region truncation within amino acid positions equivalent to position 135-174 of SEQ ID NO:1, or
c. a combination thereof.

### DESCRIPTION OF THE FIGURES

FIG. 1A-IN illustrate characterization of human BCL10 mutations in DLBCL. FIG. 1A. BCL10 protein domain and the locations of all BCL10 mutations in DLBCL identified and reported from literature and open database. FIG. 1B. Proportion of mutation types among BCL10 mutant patients. FIG. 1C. Cell of origin classification of BCL10 mutant DLBCL patients. FIG. 1D. Quantile-quantile plot showing the p-values for BCL10 SNVs across 243 ABC-DLBCL patients. FIG. 1E. NF-κB activity measured by NF-κB-RE-luciferase reporter 24 h post transfection of different BCL10 mutations into 293T cells. Luciferase activity was normalized to expression level of each mutation. EV, empty vector. Q-Actin was used as internal control. ****p<0.0001. FIG. 1F. NF-κB reporter activity in lymphoma cells expressing BCL10 mutations. ****p<0.0001. FIG. 1G. Statistical comparison of nuclear p65 staining scores between BCL10 WT and mutant tumors in TMA for DLBCL patient (n=298). ****Mann-Whitney p<0.0001. FIG. 1H. Representative images of p65 immunohistochemistry staining in BCL10 WT and mutant DLBCLs in FIG. 1G. Images were taken under magnification of 400×. Black arrowheads point to examples of p65 nuclear staining. FIG. 1. Distribution of BCL10 mutant patients using LymphGen subtypes. FIG. 1J. BCL10 mutation rate in DLBCL subtypes classified by LymphGen in a cohort of 574 patients (Staudt). FIG. 1K. Expression level of BCL10 in RNAseq dataset for DLBCL patients categorized by LymphGen. FIG. 1L. Quantile-quantile plot showing the p-values for BCL10 SNVs across all the 574 DLBCL patients. FIG. 1M. Validation of ABC-DLBCL NF-κB reporter cell lines. NF-κB reporter cell lines stably expressing NF-κB response elements followed by luciferase reporter were treated with different doses of MALT1 inhibitor (C3) or BKT inhibitor (ibrutinib). NF-κB activity was assayed 24 h after treatment. Error bars indicate SEM with 4 biological replicates. ***p<0.001; ****p<0.0001. FIG. 1N. NF-κB activity in TMD8 cells expressing BCL10 mutations. Left, TMD8 NF-κB reporter cells were stably expressing either WT or mutant forms of BCL10 and collected for WB to evaluate NF-κB activation. Right, densitometry of IκBα in the left.

FIG. 2A-2D illustrate that missense and truncated BCL10 mutant proteins enabled faster BCL10 polymerization rates. FIG. 2A illustrates the domain organization of MBP-BCL10 constructs. FIG. 2B shows images illustrating the concentration for polymerization of WT, E140X and R58Q based on confocal images of BCL10 filaments formed at concentration range between 0.1 μM-1 μM, imaged 4 h after MBP cleavage. Scale bar 5 μm. FIG. 2C shows images illustrating confocal time lapse of WT, E140X and R58Q interactions imaged at 1 μM for 30 min. E140X exhibits the fastest polymerization rate in comparison to WT and R58Q. Scale bar 5 μm. FIG. 2D shows confocal images of human diffuse large B-cell lymphoma (HBL1) cells stably expressing Flag tagged WT, E140X and R58Q, visualized by IF for BCL10 and DNA (Hoechst dye, blue).

FIG. 3A-3G illustrates the structure of BCL10 filamentation. FIG. 3A shows negative stained electron microscopy (EM) micrographs of WT, R58Q and E140X filaments. R58Q formed a mixture of thin (10 nm) and thick (20 nm). Whereas E140X formed only thin filaments similar to WT. Scale bar 100 nm. FIG. 3B shows representative 2D classes of BCL10 R58Q thin and thick filaments as observed by CryoEM. FIG. 3C illustrates the CryoEM structures of BCL10 R58Q at 4.6 Å. Left, BCL10 R58Q filament fitted into the CryoEM map. Right, BCL10 R58Q 16mer filament in which each subunit is colored differently. FIG. 3D illustrates the structure of the BCL10 R58Q monomer. The 58Q residue is labeled as a stick to the left. FIG. 3E illustrates the structure of the BCL10 R58Q layer, showing 58Q residues facing each other for stabilizing Type III intrastrand Interface (left). Zoom in of BCL10 Q58 fitted into cryoEM density (right). FIG. 3F illustrates a potential hydrogen bonding network formed by the Q58 side chain of one protomer and the carbonyl oxygen of T59 from the next protomer in the helical spiral, shown on 4 consecutive R58Q subunits in the filament (left), and as a zoom-in view (right). These interactions stabilize the Type III intrastrand Interface. FIG. 3G graphically illustrates filament thermal stability assay performed by thermal shift assay for WT, E140X and R58Q purified filaments. E140X and R58Q filaments showed a significant shift of 2.1° C. and 4.2° C., respectively in comparison to WT filaments.

FIG. 4A-4M illustrate the structure of BCL10^{E140X} filamentation. FIG. 4A shows immunoblots illustrating BCL10 interactions performed by co-immunoprecipitating with anti-Flag antibody in Raji cells overexpressing either Flag-BCL10^{WT} or Flag-BCL10^{mutant} protein. Samples were blotted for anti-Flag, anti MALT1 and anti-CARD11. Input was loaded with 1% of total cell lysate used for IP. Anti-IgG antibody was used as negative control for ColP. FIG. 4B illustrates the domain organization of MBP-human BCL10 construct mapped with MALT1 previously defined and new binding sites. FIG. 4C illustrates the domain organization of human MALT1 construct. FIG. 4D shows SDS-PAGE of MALT1 (Ig1-Ig2) pulldown by His-tagged BCL10 (165-233) (left) and His-tagged BCL10 (116-164) (right). * indicates a contaminant. FIG. 4E shows SDS-PAGE of MALT1 (Ig1-Ig2) pulldown by different truncations of His-tagged BCL10. FIG. 4F shows negative stained EM micrographs of purified BCL10 WT filaments alone and with MALT1 (left) in comparison to BCL10 E140X filaments alone and with MALT1 filaments (right), resulting in similar filaments. Scale bar 100 nm. FIG. 4G illustrates CryoEM structures of BCL10 E140X-MALT1 DD filament at 4.3 Å fitted into the cryoEM density map (left). The 4.3 Å structure is similar to the previous published BCL10 WT CARD-MALT1 DD structure at 4.9 Å (right). However, BCL10 E140X-MALT1 DD shows improved density for MALT1 DD domain. FIG. 4H illustrates a CryoEM structure of BCL10 E140X CARD and MALT1 DD (cyan) filament, emphasizing EM density for MALT1 DD. FIG. 4I shows a structure of monomeric BCL10 E140X CARD-MALT1 DD (cyan) align to published monomeric BCL10 WT CARD-MALT1 DD. FIG. 4J shows Western blots of gel filtration fractions of HBL1 cells stably expressing Flag tagged BCL10 WT, E140X and R58Q. Fractions were blotted for anti-Flag and anti-MALT1. BCL10 E140X formed highly ordered oligomers co-migrated with MALT1, isolated from the void fractions. FIG. 4K graphically illustrates that MALT1 inhibits BCL10 filament formation through at least one BCL10 C-terminal binding site. Quenching polymerization was measured for purified Alexa488 labeled BCL10 WT, E140X and R58Q at 3 μM in the presence of increasing amounts of MALT1 (0 μM, 1.5 μM, 3 μM, 6 μM and 12 μM). The assay was initiated upon addition of the 3C protease, in order to remove MBP tag from BCL10 WT, E140X and R58Q for allowing filament polymerization. Quenching was monitored for 2 h with 30 sec intervals by using Neo Biotek plate reader and performed with 3 biological replicates. Titration of increasing doses of MAL T1 suppressed filament polymerization of fluorescently labeled BCL10 WT and R58Q. However, increasing doses of MALT1 had very little effect on E140X filament polymerization. FIG. 4L graphically illustrates MALT1 activity measured in Raji GloSensor reporter cells. Raji MALT1 GloSensor cell lines were stably expressing wildtype and mutant BCL10, and MALT1 protease activity was measured once the antibiotic selection was done. GloSensor activity was normalized to each WT cells. Error bars indicate SEM with 4 biological replicates. ***p<0.001; ****p<0.0001. FIG. 4M graphically illustrates MALT1 activity measured in ABC-DLBCL GloSensor reporter cells. HBL1, TMD8 and OCI-Ly10 MALT1 GloSensor cell lines were stably expressing wildtype and mutant BCL10, and MALT1 protease activity was measured once the antibiotic selection was done. GloSensor activity was normalized to each WT cells. Error bars indicate SEM with 4 biological replicates. *p<0.05; ***p<0.001; ****p<0.0001; ns, not significant.

FIG. 5A-5D illustrate that the BCL10 mutations are less dependent on upstream CARD11. FIG. 5A graphically illustrates the viability of HBL1 lymphoma cell lines transduced to express shRNA targeting CARD11 with two independent hairpins or non-targeting control. The indicated lines stably expressing wildtype and mutant BCL10, were transduced with lentiviruses expressing CARD11 shRNA along with YFP. The relative number of YFP+ live cells was plotted by normalizing them to Day 4 (the YFP+ peak). ***p<0.001; ****p<0.0001. FIG. 5B graphically illustrates the MALT1 activity using the MALT1 GloSensor reporter cells with CARD11 knockdown. The indicated MALT1 GloSensor cell lines were stably expressing wildtype and mutant BCL10, then transduced with lentiviruses expressing non-targeting or 2 independent CARD11/hairpins coexpressing YFP reporter. At day 4, cells were harvested for MALT1 activity assay. Error bars indicate SEM with 4 biological replicates. ***p<0.001; ****p<0.0001; ns, not significant. FIG. 5C graphically illustrates the NF-κB activity in lymphoma reporter cells with shCARD11. The HBL1 NF-κB reporter cells were stably expressing wildtype and mutant BCL10, then transduced with lentiviruses expressing non-targeting or 2 independent CARD11 hairpins coexpressing YFP reporter. NF-κB activity was measured 72 h post-transduction. Error bars indicate SEM with 4 biological replicates. ****p<0.0001. FIG. 5D graphically illustrates the viability of OCI-Ly10 lymphoma cell lines transduced to express shRNA targeting CARD11 with two independent hairpins or non-targeting control. The indicated lines stably expressing wildtype and mutant BCL10, were transduced with lentiviruses expressing CARD11 shRNA along with YFP. The relative number of YFP+ live cells was plotted by normalizing to Day 3 (the YFP+ peak).

FIG. 6A-6K illustrate that BCL10 gain-of-function mutant lymphomas are resistant to upstream Bruton's tyrosine kinase (BTK) inhibitors. FIG. 6A illustrates growth inhibition assays of human diffuse large B-cell lymphoma (HBL1) cells and OCI-Ly10 lymphoma cells expressing wild type or mutant BCL10 protein in response to the BTK inhibitor Acalabrutinib. X axis, concentration of compound (M); Y axis, inhibition of cell growth normalized to vehicle treated cells. *p<0.05; **p<0.01; ****p<0.0001; ns, not significant. FIG. 6B illustrates growth inhibition assays of human diffuse large B-cell lymphoma (HBL1) cells and OCI-Ly10 lymphoma cells expressing wild type or mutant BCL10 protein in response to the BTK inhibitor Zanubrutinib. X axis, concentration of compound (M); Y axis, inhibition of cell growth normalized to vehicle treated cells. *p<0.05; **p<0.01; ****p<0.0001; ns, not significant. FIG. 6C illustrates growth inhibition assays of human diffuse large B-cell lymphoma (HBL1) cells and OCI-Ly10 lymphoma cells expressing wild type or mutant BCL10 protein in response to the BTK inhibitor Ibrutinib. X axis, concentration of compound (M); Y axis, inhibition of cell growth normalized to vehicle treated cells. *p<0.05; **p<0.01; ****p<0.0001; ns, not significant. FIG. 6D illustrates luciferase activity measured in the MALT1 GloSensor reporter cell lines upon BTK inhibitor Acalabrutinib treatment. The indicated MALT1 GloSensor reporter lines were stably expressing wildtype and mutant BCL10, and then treated with BTK inhibitors of different range (10-0.3 nM). NF-κB activity was performed 24 h after treatment. Error bars indicate SEM with 4 biological replicates. *p<0.05; **p<0.01; ***p<0.001; ****p<0.0001; ns, not significant. FIG. 6E illustrates luciferase activity measured in the MALT1 GloSensor reporter cell lines upon BTK inhibitor Zanubrutinib treatment. The indicated MALT1 GloSensor reporter lines were stably expressing wildtype and mutant BCL10, and then treated with BTK inhibitors of different range (10-0.3 nM). NF-κB activity was performed 24 h after treatment. Error bars indicate SEM with 4 biological replicates. *p<0.05; **p<0.01; ***p<0.001; ****p<0.0001; ns, not significant. FIG. 6F illustrates luciferase activity measured in the MALT1 GloSensor reporter cell lines upon BTK inhibitor Ibrutinib treatment. The indicated MALT1 GloSensor reporter lines were stably expressing wildtype and mutant BCL10, and then treated with BTK inhibitors of different range (10-0.3 nM). NF-κB activity was performed 24 h after treatment. Error bars indicate SEM with 4 biological replicates. *p<0.05; **p<0.01; ***p<0.001; ****p<0.0001; ns, not significant. FIG. 6G illustrates the experimental design for evaluating different BCL10 xenografts with ibrutinib treatment. FIG. 6H graphically illustrates tumor growth (volume) for xenografts of HBL1 cells expressing WT and mutant BCL10 treated with vehicle and ibrutinib (n=5-˜6/group). Mice were treated orally with 25 mg/kg ibrutinib once per day for 24 consecutive days. FIG. 6I graphically illustrates area under the curve (AUC) of the data shown in FIG. 6H. AUC was calculated with Prism. FIG. 6J graphically illustrates tumor weight measured at the endpoint of tumors formed from xenografts of HBL1 cells expressing WT and mutant BCL10 (n=5-6/group). Mice were treated orally with 25 mg/kg ibrutinib once per day for 24 consecutive days. FIG. 6K shows representative photos of tumors harvested at endpoint from mice with xenografts of HBL1 cells expressing WT and mutant BCL10. Mice were treated orally with 25 mg/kg ibrutinib once per day for 24 consecutive days.

FIG. 7A-7K illustrate that subjects with BCL10 truncating mutant lymphomas are hypersensitive to MALT1 protease inhibitors. FIG. 7A graphically illustrates growth inhibition of human diffuse large B-cell lymphoma (HBL1) cells and OCI-Ly10 lymphoma cells expressing wild type or mutant BCL10 proteins in response to MALT1 inhibitor C3. X axis, concentration of compound (M); Y axis, inhibition of cell growth normalized to vehicle treated cells. **p<0.01; ***p<0.001; ****p<0.0001; ns, not significant. FIG. 7B graphically illustrates growth inhibition of human diffuse large B-cell lymphoma (HBL1) cells and OCI-Ly10 lymphoma cells expressing wild type or mutant BCL10 proteins in response to MALT1 inhibitor MLT-748. X axis, concentration of compound (M); Y axis, inhibition of cell growth normalized to vehicle treated cells. **p<0.01; ***p<0.001; ****p<0.0001; ns, not significant. FIG. 7C graphically illustrates growth inhibition of human diffuse large B-cell lymphoma (HBL1) cells and OCI-Ly10 lymphoma cells expressing wild type or mutant BCL10 proteins in response to MALT1 inhibitor JNJ-67690246. X axis, concentration of compound (M); Y axis, inhibition of cell growth normalized to vehicle treated cells. **p<0.01; ***p<0.001; ****p<0.0001; ns, not significant. FIG. 7D graphically illustrates luciferase activity measured in the MALT1 GloSensor reporter cell lines treated with MALT1 inhibitor C3. Indicated MALT1 GloSensor reporter lines were stably expressing wildtype and mutant BCL10, and then treated with MALT1 inhibitors of different range (10-0.3 uM). NF-κB activity was performed 24 h after treatment. Error bars indicate SEM with 4 biological replicates. FIG. 7E graphically illustrates luciferase activity measured in the MALT1 GloSensor reporter cell lines treated with MALT1 inhibitor MLT-748. Indicated MALT1 GloSensor reporter lines were stably expressing wildtype and mutant BCL10, and then treated with MALT1 inhibitors of different range (10-0.3 uM). NF-κB activity was performed 24 h after treatment. Error bars indicate SEM with 4 biological replicates. FIG. 7F graphically illustrates luciferase activity measured in the MALT1 GloSensor reporter cell lines treated with MALT1 inhibitor JNJ-67690246. Indicated MALT1 GloSensor reporter lines were stably expressing wildtype and mutant BCL10, and then treated with MALT1 inhibitors of different range (10-0.3 uM). NF-κB activity was performed 24 h after treatment. Error bars indicate SEM with 4 biological replicates. FIG. 7G illustrates the experimental design of mice bearing xenografts treated with JNJ-67690246. FIG. 7H graphically illustrates tumor growth of xenografts of HBL1 cells expressing WT and mutant BCL10 upon treatment with vehicle and JNJ-67690246 (n=3˜6/group). Mice were treated orally with 100 mg/kg twice per day for 19 consecutive days. FIG. 7I graphically illustrates area under the curve (AUC) from the data shown in FIG. 7H. AUC was calculated with Prism. *p<0.05. FIG. 7J graphically illustrates tumor weight measured at the endpoint of the studies described in FIG. 7G-7I. *p<0.05. FIG. 7K shows representative photos of tumors harvested at endpoint of the studies described in FIG. 7G-7J.

FIG. 8A-8H illustrate characterization of human BCL10 mutations in DLBCL. FIG. 8A. BCL protein domain and the locations of all BCL10 mutations in DLBCL identified and reported from literature and open database. FIG. 8B. Proportion of mutation types among BCL10 mutant patients. FIG. 8C. Cell of origin classification of BCL10 mutant DLBCL patients. FIG. 8D. Quantile-quantile plot showing the p-values for BCL10 SNVs across 243 ABC-DLBCL patients. FIG. 8E. NF-κB activity measured by NF-xB-RE-luciferase reporter 24 h post transfection of different BCL10 mutations into 293T cells. Luciferase activity was normalized to expression level of each mutation. EV, empty vector. Q-Actin was used as internal control. ****p<0.0001. FIG. 8F. NF-κB reporter activity in lymphoma cells expressing BCL10 mutations. ****p<0.0001. FIG. 8G. Statistical comparison of nuclear p65 staining scores between BCL10 WT and mutant tumors in TMA for DLBCL patient (n=298). ****Mann-Whitney p<0.0001. FIG. 8H. Representative images of p65 immunohistochemistry staining in BCL10 WT and mutant DLBCLs in (G). Images were taken under magnification of 400×. Black arrowheads point to examples of p65 nuclear staining.

FIG. 9A-9D illustrate representative missense and truncating mutant BCL10 enabled faster polarization rate. FIG. 9A. Domain organization of MBP-BCL10 construct. FIG. 9B. Concentration determination of WT, E140X and R58Q based on confocal images of BCL10 filaments formed at concentration range between 0.1 μM-1 M, imaged 4 h after MBP cleavage. Scale bar 5 μm. FIG. 9C. Confocal time lapse of WT, E140X and R58Q imaged at 1 M for 30 min. E140X exhibits the fastest polymerization rate in comparison to WT and R58Q. Scale bar 5 μm. FIG. 9D. Confocal images of HBL1 cells stably expressing Flag tagged WT, E140X and R58Q, visualized by IF for BCL10 and DNA (Hoechst dye, blue).

FIG. 10A-10G illustrate cryoEM structure of BCL10^{R58Q} filamentation. FIG. 10A. Negative stained EM micrographs of WT, R58Q and E140X filaments. R58Q formed a mixture of thin (10 nm) and thick (20 nm). Whereas E140X formed only thin filaments similar to WT. Scale bar 100 nm. FIG. 10B. Representative 2D classes of BCL10 R58Q thin and thick filaments. FIG. 10C. CryoEM structure of BCL10 R58Q at 4.6 A. Left, BCL10 R58Q filament fitted into the CryoEM map. Right, BCL10 R58Q 16mer filament in which each subunit is colored differently. FIG. 10D. BCL10 R58Q monomer. 58Q residue is labeled as stick. FIG. 10E. BCL10 R58Q layer, showing 58Q residues facing each other for stabilizing Type III 9 intrastrand Interface (left). Zoom in of BCL10 058 fitted into cryoEM density (right). FIG. 10F. Potential hydrogen bonding network formed by the Q58 side chain of one protomer and the carbonyl oxygen of T59 from the next protomer in the helical spiral, shown on consecutive R58Q subunits in the filament (left), and as a zoom-in view (right). These interactions stabilize the Type III intrastrand Interface. FIG. 10G. Filament thermal stability assay performed by thermal shift assay for WT, E140X and R58Q purified filaments. E140X and R58Q filaments showed a significant shift in 2.1° C. and 4.2° C., respectively in comparison to WT filaments.

FIG. 11A-11K illustrate cryoEM structure of BCL10^{E140X} filamentation. FIG. 11A. Immunoblot analysis of BCL10 interactors performed by co-immunoprecipitating with anti-Flag antibody in Raji cells overexpressing either Flag-BCL10^{WT} or Flag-BCL10^{mutant} protein. Samples were blotted for anti-Flag, anti MALT1 and anti-CARD11. Input was loaded with 1% of total cell lysate used for IP. Anti-IgG antibody was used as negative control for ColP. FIG. 11B. Domain organization of MBP-human BCL10 construct mapped with MALT1 previously defined and new binding sites. FIG. 11C. Domain organization of human MALT1 construct. FIG. 11D. SDS-PAGE of MALT1 (Ig1-Ig2) pulldown by His-tagged BCL10 (165-233) (left) and His-tagged BCL10 (116-164) (right). * indicates a contaminant. FIG. 11E. SDS-PAGE of MALT1 (Ig1-Ig2) pulldown by different truncations of His-tagged BCL10. FIG. 11F. Negative stained EM micrographs of purified BCL10 WT filaments alone and with MALT1 (left) in comparison to BCL10 E140X filaments alone and with MALT1 filaments (right), 31 resulted in similar filaments. Scale bar 100 nm. FIG. 11G. CryoEM structure of BCL10 E140X-MALT1 DD filament at 4.3 Å fitted into the cryoEM density map (left). The 4.3 Å structure is similar to the previous published BCL10 WT CARD-MALT1 DD structure at 4.9 Å (right). However, BCL10 E140X-MALT1 DD shows improved density for MALT1 DD domain. FIG. 11H. CryoEM structure of BCL10 E140X CARD and MALT1 DD (cyan) filament, emphasizing EM density for MALT1 DD. FIG. 11I. Monomeric BCL10 E140X CARD-MALT1 DD (cyan) align to published monomeric BCL10 WT CARD-MALT1 DD. FIG. 11J. Western blot for gel filtration fractions of HBL1 cells stably expressing Flag tagged BCL10 WT, E140X and R58Q. Fractions were blotted for anti-Flag and anti-MALT1. BCL10 E140X formed highly ordered oligomers co-migrated with MALT1, isolated from the void fractions. FIG. 11K. MALT1 inhibits BCL10 filament formation through BCL10 C-terminal binding site. Quenching polymerization was measured for purified Alexa488 labeled BCL10 WT, E140X and R58Q at 3 μM in the presence of increasing amounts of MALT1 (0 μM, 1.5M, 3M, 6M and 12 μM). The assay was initiated upon addition of the 3C protease, in order to remove MBP tag from BCL10 WT, E140X and R58Q for allowing filament polymerization. Quenching was monitored for 2 h with 30 sec intervals by using Neo Biotek plate reader and performed with 3 biological replicates. Titration of increasing doses of MALT1 suppressed filament polymerization of fluorescently labeled BCL10 WT and R58Q. However, increasing doses of MALT1 had very little effect on E140X filament polymerization.

FIG. 12A-12C illustrate BCL10 mutations are less dependent on upstream CARD11. FIG. 12A. Viability of HBL1 lymphoma cell lines transduced to express shRNA targeting CARD11 with two independent hairpins or non-targeting control. The indicated lines stably expressing wildtype and mutant BCL10, were transduced with lentiviruses expressing CARD11 shRNA along with YFP. The relative number of YFP+ live cells was plotted by normalizing them to Day 4 (the YFP+ peak). ***p<0.001; ****p<0.0001. FIG. 12B. MALT1 activity using the MALT1 GloSensor reporter cells with CARD11 knockdown. The indicated MALT1 GloSensor cell lines were stably expressing wildtype and mutant BCL10, then transduced with lentiviruses expressing non-targeting or 2 independent CARD11 hairpins coexpressing YFP reporter. At day 4, cells were harvested for MALT1 activity assay. Error bars indicate SEM with 4 biological replicates. ***p<0.001; ****p<0.0001; ns, not significant. FIG. 12C. NF-xB activity in lymphoma reporter cells with shCARD11. The HBL1 NF-xB reporter cells were stably expressing wildtype and mutant BCL10, then transduced with lentiviruses expressing non-targeting or 2 independent CARD11 hairpins coexpressing YFP reporter. NF-κB activity was measured 72 h post-transduction. Error bars indicate SEM with 4 biological 7 replicates. ****p<0.0001.

FIG. 13A-13K illustrate BCL10 Gain-of-function mutant lymphomas are resistant to upstream BTK inhibitors. FIG. 13A-13C. Growth inhibition assay of lymphoma cells expressing WT or mutant BCL10 in response to BTK inhibitors. X axis, concentration of compound (M); Y axis, inhibition of cell growth normalized to vehicle treated cells. *p<0.05; **p<01; ****p<0.0001; ns, not significant. FIG. 13D-13F. Luciferase activity measured in the MALT1 GloSensor reporter cell lines with BTK inhibitor treatment. Indicated MALT1 GloSensor reporter lines were stably expressing wildtype and mutant BCL10, and then treated with BTK inhibitors of different range (10-0.3 nM). NF-κB activity was performed 24 h after treatment. Error bars indicate SEM with 4 biological replicates. *p<0.05; **p<0.01; ***p<0.001; ****p<0.0001; ns, not significant. FIG. 13G. Experimental design of xenografts with ibnrtinib treatment. FIG. 13H. Tumor growth curve for xenografts of HBL1 cells expressing WT and mutant BCL10 treated with vehicle and ibrutinib (n=5-6/group). Mice were treated orally with 25 mg/kg ibrutinib once per day for 24 consecutive days. FIG. 1. Analysis of area under the curve (AUC) in h. AUC is calculated with Prism. FIG. J. Tumor weight measured at the endpoint. FIG. K. Representative photos of tumors harvested at endpoint.

FIG. 14A-14K illustrate BCL10 truncating mutant lymphomas are hypersensitive to MALT1 protease inhibitors. FIG. 14A-14C. Growth inhibition assay of lymphoma cells expressing WT or mutant BCL10 in response to MALT1 inhibitors. X axis, concentration of compound (M); Y axis, inhibition of cell growth normalized to vehicle treated cells. **p<0.01; ***p<0.001; ****p<0.0001; ns, not significant. FIG. 14D-14F. Luciferase activity measured in the MALT1 GloSensor reporter cell lines with MALT1 inhibitor treatment. Indicated MALT1 GloSensor reporter lines were stably expressing wildtype and mutant BCL10, and then treated with MALT1 inhibitors of different range (10-0.3 uM). NF-xB activity was performed 24 h after treatment. Error bars indicate SEM with 4 biological replicates. FIG. 14G. Experimental design of xenografts with JNJ-67690246 treatment. FIG. 14H. Tumor growth curve for xenografts of HBL1 cells expressing WT and mutant BCL10 treated with vehicle and JNJ-67690246 (n=3-6/group). Mice were treated orally with 100 mg/kg twice per day for 19 consecutive days. FIG. 14I. Analysis of area under the curve (AUC) in h. AUC is calculated with Prism. *p<0.05. FIG. 14J. Tumor weight measured at the endpoint. *p<0.05. FIG. 14K. Representative photos of tumors harvested at endpoint.

FIG. 15A-15G illustrate mutation and gene expression analysis of BCL10 in DLBCL patients. FIG. 15A. BCL10 mutation rate in DLBCL subtypes classified by cell-of-origin in a cohort of 574 patients (Staudt). FIG. 15B. Distribution of BCL10 mutant patients using LymphGen subtypes. FIG. 15C. BCL10 mutation rate in DLBCL subtypes classified by LymphGen in a cohort of 574 patients (Staudt). FIG. 15D. Expression level of BCL10 in RNAseq dataset for DLBCL patients categorized by LymphGen. FIG. 15E. Quantile-quantile plot showing the p-values for BCL10 SNVs across all the 574 DLBCL patients. FIG. 15F. Validation of ABC-DLBCL NF-κB reporter cell lines. NF-κB reporter cell lines stably expressing NF-κB response elements followed by luciferase reporter were treated with different doses of MALT1 inhibitor (C3) or BKT inhibitor (ibrutinib). NF-κB activity was assayed 24 h after treatment. Error bars indicate SEM with 4 biological replicates. ***p<0.001; ****p<0.0001. FIG. 15G. NF-κB activity in TMD8 cells expressing BCL10 mutations. Left, TMD8 NF-κB reporter cells were stably expressing either WT or mutant forms of BCL10 and collected for WB to evaluate NF-κB activation. Right, densitometry of 1B in the left.

FIG. 16 shows SDS-PAGE void and monomer gel filtration fractions of purified MBP tagged WT, R58Q and E140X.

FIG. 17A-17E show CryoEM data processing and structure of the BCL10^{R58Q} filament. FIG. 17A. Raw cryoEM micrograph of BCL10 R58Q, showing thick (red) and thin (blue) filaments. Each thick filament is composed of two thin filaments bundled together. FIG. 17B. Gold standard Fourier shell correlation (FSC) of BCL10 R58Q filament, which gave an overall resolution of 4.6 Å. FIG. 17C. The BCL10 WT CARD structure (6BZE) fitted into its cryoEM density map at 4.0 Å showing the poor density in the region of R58. FIG. 17D. Negative staining EM micrographs of WT, R58Q and R58E filaments. R58E formed similar filaments as the WT. Scale bar 100 nm. FIG. 10E Approximate concentration determination of R58E based on confocal images of BCL10 filaments formed at a concentration range between 0.1 M-1 M, imaged 4 h after MBP cleavage. Scale bar 5 μm.

FIG. 18A-18G illustrate BCL10^{E140X} is mostly present in oligomerized fraction, with correlated MALT1 enrichment and MALT1 protease activity in lymphoma cells. FIG. 18A. Gel filtration profile of MALT1 (207-309)-BCL10 (165-233) complex. FIG. 18B. Overexpression efficiency of WT and mutant BCL10 in HBL1 lymphoma cells. FIG. 18C. Western blot for sucrose gradient fractions of HBL1 cells stably expressing Flag tagged BCL10 WT, R58Q and E140X. Fractions were blotted for anti-Flag and anti-MALT1. Consistent with gel filtration results, BCL10 E140X formed highly ordered oligomers comigrated with MALT1. FIG. 18D. MALT1 activity measured in Raji GloSensor reporter cells. Raji MALT1 GloSensor cell lines were stably expressing wildtype and mutant BCL10, and MALT1 protease activity was measured once the antibiotic selection was done. GloSensor activity was normalized to each WT cells. Error bars indicate SEM with 4 biological replicates. ***p<0.001; ****p<0.0001. FIG. 18E. Overexpression efficiency of WT and mutant BCL10 in Raji GloSensor reporter cells. FIG. 18F. MALT1 activity measured in ABC-DLBCL GloSensor reporter cells. HBL1, TMD8 and OCI-Ly10 MALT1 GloSensor cell lines were stably expressing wildtype and mutant BCL10, and MALT1 protease activity was measured once the antibiotic selection was done. GloSensor activity was normalized to each WT cells. Error bars indicate SEM with 4 biological replicates. *p<0.05; ***p<0.001: ****p<0.0001; ns, not significant. FIG. 18G. Overexpression efficiency of WT and mutant BCL10 in HBL1, TMD8 and OCI-Ly10 GloSensor reporter cells.

FIG. 19A-19C illustrate BCL10 mutations showed consistently less CARD11 dependency in ABC-DLBCL cells. FIG. 19A. Viability of OCI-Ly10 lymphoma cell lines transduced to express shRNA targeting CARD11 with two independent hairpins or non-targeting control. The indicated lines stably expressing wildtype and mutant BCL10, were transduced with lentiviruses expressing CARD11 shRNA along with YFP. The relative number of YFP+ live cells was plotted by normalizing to Day 3 (the YFP+ peak). FIG. 19B. Knockdown efficiency of CARD11 in HBL1 Glosensor cells expressing BCL10 and shCARD11-YFP. FIG. 19C. Knockdown efficiency of CARD11 in OCI-Ly10 cells expressing BCL10 and shCARD11-YFP.

FIG. 20A-20G illustrate BCL10 mutations confer BTK resistance in both MALT1 activity and NF-κB reporter assays. FIG. 20A. Table for IC50s of three different BTK inhibitors measured in HBL1 and OCI-Ly10 cells expressing WT and mutant BCL 10. FIG. 20B-D. MALT1 activity measured in OCI-Ly10 GloSensor reporter cells treated with BTK inhibitors. OCI-Ly10 MALT1 GloSensor cell lines were stably expressing wildtype and mutant BCL10, then treated with BTK inhibitors of different range (10-0.3 nM). Error bars indicate SEM with 4 biological replicates. *p<0.05; **p<0.01; ****p<0.0001; ns, not significant. FIG. 20E-G. NF-κB activity measured in NF-κB-RE-luciferase reporter cell lines treated with BTK inhibitors. HBL1 NF-κB reporter cells lines were stably expressing wildtype and mutant BCL10, and then treated with BTK inhibitor of different range (10-0.3 nM). NF-κB activity was performed 24 h after treatment. Error bars indicate SEM with 4 biological replicates. ***p<0.001; ****p<0.0001; ns, not significant.

FIG. 21A-211 illustrate MALT1 enzymatic activity, but not NF-κB or cell growth can be equally inhibited by different MALT1 protease inhibitors. FIG. 21A. Chemical structure of JNJ-67690246. FIG. 21B. IC50 values for JNJ-67690246 in biochemical enzymatic and cellular cytokine secretion assays. MALT1 proteolytic IC₅₀: purified recombinant MALT1 was preincubated for 50 min at RT with JNJ-67690246, substrate Ac-LRSR-AMC was then incubated for 4 h. MALT1 proteolytic activity was determined by measuring the increase of AMC fluorescence. IL-6 and IL-10 secretion IC₅₀: OCI-Ly3 cells were treated for 24 h with JNJ-67690246 or solvent (DMSO), and levels of IL-6 and IL-10 in the culture supernatant were assessed by MSD. FIG. 21C. Table for IC50 values of three different MALT1 inhibitors measured in HBL1 and OCI-Ly10 cells expressing WT and mutant BCL10. D-F. MALT1 activity measured in OCI-Ly10 GloSensor reporter cells treated with MALT1 inhibitors. OCI-Ly10 MALT1 GloSensor cells were stably expressing wildtype and mutant BCL10, then treated with MALT1 inhibitors of different range (10-0.3 uM). FIG. 21G-I. NF-κB activity measured in NF-κB-RE-luciferase reporter cell lines treated with MALT1 inhibitors. HBL1 NF-κB reporter cells lines were stably expressing wildtype and mutant BCL10, and then treated with MALT1 inhibitors of different range (10-0.3 uM). NF-xB activity was performed 24 h after treatment. Error bars indicate SEM with 4 biological replicates. p<0.05; “p<0.01; “<0.001; ****p<0.0001; ns, not significant.

### DETAILED DESCRIPTION

As described herein, subjects having lymphomas who also have BCL10 mutations can be treated more effectively with MALT1 inhibitors than other types of treatments. In some cases, the subjects are resistant to BTK inhibitors. Treatment with MALT1 inhibitors can therefore be significantly more effective than other types of treatments. Genome sequencing studies identified BCL10 gain-of-function mutations in diffuse large B-cell lymphomas (DLBCLs), and mostly within the ABC-DLBCLs. Through biochemical, structural and functional dissection of these mutations, two distinct classes of BCL10 mutations were identified: a missense (CARD) mutation and C-terminal truncations of BCL10.

A method is described herein that can involve administering at least one MALT1 inhibitor to a subject having a mutation in at least one BCL10 allele.

Also described herein are methods of identifying subjects who can benefit from treatment with at least one MALT1 inhibitor, the method involving screening the subjects to identify which subjects, if any, have at least one mutation in at least one BCL10 allele.

The BCL10 mutation(s) can be missense mutations, deletions, or insertions in the BCL10 coding region of at least one BCL10 allele. In some cases, the mutation is in a BCL10 CARD domain. In some cases, the mutation is a BCL10 C-terminal truncation or BCL10 C-terminal deletion in the BCL10 coding region. Subjects with at least mutation in a coding region of at least one BCL10 allele can be treated with one or more MALT1 inhibitors.

In some cases, at least one of the mutations is in the BCL10 CARD domain such as at an amino acid position equivalent to position 58 of SEQ ID NO:1, or the mutation includes an amino acid position equivalent to position 58. In some cases, the C-terminal truncation or C-terminal deletion in a coding region of at least one BCL10 allele is within amino acid positions equivalent to position 135-174 of SEQ ID NO:1. The BCL10 mutations can be at positions 58 and 140 et seq.

Subjects can be evaluated for treatment with one or more MALT1 inhibitors by a variety of methods such as genomic sequencing, polymerase chain reaction (PCR) analysis, restriction fragment length polymorphism (RFLP) analysis, mRNA size analysis, protein sequencing, antibody detection of BCL10 mutant proteins, use of primers/probes specific for truncation mutant BCL10 proteins, and the like.

### B-Cell Lymphoma/Leukemia 10 (BCL10)

BCL10 is composed of an N-terminal caspase activation and recruitment domain (CARD) domain, and a long C-terminal unstructured region containing a distal Ser and Thr rich region. Structure guided studies have shown that the BCL10 filament polymerizes in a unidirectional manner through CARD-CARD interactions, providing a surface for cooperative binding of MALT1 through its N-terminal Death Domain. Upon BCL10 filament binding, MALT1 immediately dimerizes and incorporates TRAF6 to form a higher ordered assembly leading to all-or-none activation of downstream pathways including NF-κB and JNK. Binding to BCL10 also activates MALT1 paracaspase activity and cleavage of substrate proteins. BCL10 filament formation is dynamic in activated T lymphocytes and precisely regulated by disassembly and degradation through BCL10 K63 polyubiquitination and p62-dependent selective autophagy-lysosomal proteolysis system. Hence dynamic BCL10 filament turnover is needed to precisely tune its effect on downstream signaling pathways such as NF-κB.

Chronic active NF-κB signaling is a hallmark of highly aggressive activated B cell-like diffuse large B-cell lymphomas (ABC-DLBCLs), due to somatic mutations of B-cell receptors (BCRs) and Toll-like receptor (TLR) subunits such as CD79b and MYD88 (Young et al. SeminHematol 52:77-85 (2015); Davis et al. Nature 463:88-92 (2010); Ngo et al., Nature 470:115-119 (2011)), as well as activating mutations of CARD11 and amplifications of MALT1 (Lenz et al., Science 319:1676-1679 (2008); Sanchez-Izquierdo et al., Blood 101:4539-4546 (2003); Vicente-Duenas et al., Proc Natl Acad Sci USA 109:10534-10539 (2012)). Collectively these mutations induce chronic activation of the CARD11-BCL10-MALT1 (CBM) complex to maintain robust and sustained NF-κB and other downstream pathway activation. The involvement of these signaling pathways in highly aggressive tumors has inspired development of targeted therapies disrupting oncogenic BCR/TLR activity.

However, the position where mutations happen in the BCR pathway may be important for assigning potential precision therapy to patients. For example, mutations in the most upstream BCR proteins like CD79B confer sensitivity to BTK inhibitors, whereas downstream mutations like PLCγ2 and CARD11 confer resistance (Wilson et al. Nat Med 21:922-926 (2015); Hendricks et al., Nat Rev Cancer 14:219-232 (2014); Woyach et al., N Engl J Med 370:2286-2294 (2014); Caeser et al., JCO Precis Oncol 5:145-152 (2021)). Hence mechanistic study of oncogenic mutations is beneficial to guide targeted therapy in B cell lymphomas.

Aberrant CBM function has been shown to play roles in diseases such as B-cell lymphoma and auto-immunity. Upon antigen receptor engagement, the CARD11 subunit is phosphorylated by protein kinase C (PKC), which activates its function by reducing interaction of its auto-inhibitory coiled coil domain to its CARD domain. The activated form CARD11 then can interact with BCL10 and facilitate formation of large macromolecular filaments, providing a large scaffold for binding and activation of MALT1, which is the enzymatic paracaspase subunit of the CBM complex that results in further downstream activation of a variety of effector molecules. Like other supramolecular organizing center (SMOC) mediated signaling transduction, such as toll-like receptor (TLR) triggering Myddosome, RIG-1 like receptor sensing intracellular viral RNA and activating mitochondrial antiviral signaling protein (MAVS) filament formation, the BCL10 filament formation is also important for BCR/TCR signaling amplification and robust downstream NF-κB activation.

The human BCL10 gene is located on chromosome 1 (location 1p22.3; NC_000001.11 (85265776 . . . 85276632, complement; NC_060925.1 (85106896 . . . 85117748, complement).

A sequence for isoform 1 of human BCL10 is shown below (NCBI NP_003912.1; SEQ ID NO:1).

| | |
|---|---|
| 1 | MEPTAPSLTE EDLTEVKKDA LENIRVYLCE KIIAERHFDH |
| 41 | LRAKKILSRE DTEEISCRTS SRKRAGKLLD YLQENPKGLD |
| 81 | TLVESIRREK TQNFLIQKIT DEVLKLRNIK LEHLKGLKCS |
| 121 | SCEPFPDGAT NNLSRSNSDE SNFSEKLRAS TVMYHPEGES |
| 161 | STTPFFSTNS SLNLPVLEVG RTENTIFSST TLPRPGDPGA |
| 201 | PPLPPDLQLE EEGTCANSSE MFLPLRSRTV SRQ |

A cDNA encoding human BCL10 protein is shown below as SEQ ID NO:2.

| | |
|---|---|
| 1 | ATGGAGCCCA CCGCACCGTC CCTCACCGAG GAGGACCTCA |
| 41 | CTGAAGTGAA GAAGGACGCC TTAGAAAATT TACGTGTATA |
| 81 | CCTGTGTGAG AAAATCATAG CTGAGAGACA TTTTGATCAT |
| 121 | CTACGTGCAA AAAAAATACT CAGTAGAGAA GACACTGAAG |
| 161 | AAATTTCTTG TCGAACATCA AGTAGAAAAA GGGCTGGAAA |
| 201 | ATTGTTAGAC TACTTACAGG AAAACCCAAA AGGTCTGGAC |
| 241 | ACCCTTGTTG AATCTATTCG GCGAGAAAAA ACACAGAACT |
| 281 | TCCTGATACA GAAGATTACA GATGAAGTGC TGAAACTTAG |
| 321 | AAATATAAAA CTAGAACATC TGAAAGGACT AAAATGTAGC |
| 361 | AGTTGTGAAC CTTTTCCAGA TGGAGCCACG AACAACCTCT |
| 401 | CCAGATCAAA TTCAGATGAG AGTAATTTCT CTGAAAAACT |
| 441 | GAGGGCATCC ACTGTCATGT ACCATCCAGA AGGAGAATCC |
| 481 | AGCACGACGC CCTTTTTTTC TACTAATTCT TCTCTGAATT |
| 521 | TGCCTGTTCT AGAAGTAGGC AGAACTGAAA ATACCATCTT |
| 561 | CTCTTCAACT ACACTTCCCA GACCTGGGGA CCCAGGGGCT |
| 601 | CCTCCTTTGC CACCAGATCT ACAGTTAGAA GAAGAAGGAA |
| 641 | CTTGTGCAAA CTCTAGTGAG ATGTTTCTTC CCTTAAGATC |
| 681 | ACGTACTGTT TCACGACAAT GA |

Another cDNA encoding this human BCL10 protein is shown below as SEQ ID NO:3.

| | |
|---|---|
| 1 | TGCCTGCGCC TGAGCCTCTA CGAGAGGGAA GGAACGCTGC |
| 41 | TCCGAGCTCC GCGTCGCGTC GCGTAGATTC GCGTCGCCGT |
| 81 | CGACCTCAGA GGCGGGGCCG GAAGCGCTAC GGTTTGACCC |
| 121 | CCGAGTCCCT CTGTTCCCGA AGGGGCGGCC GTCTTTCTCC |
| 161 | CGACCCGCTC CGCCTCCTCT CCTTCTTCCC CATTACCCGG |
| 201 | AGGCCGAAGC CCCCAGCCAG GGCGGGGCGG CGCAGCCCGA |
| 241 | GCTCCCGGAC CCGGAAGAAG CGCCATCTCC CGCCTCCACC |
| 281 | ATGGAGCCCA CCGCACCGTC CCTCACCGAG GAGGACCTCA |
| 321 | CTGAAGTGAA GAAGGACGCC TTAGAAAATT TACGTGTATA |
| 361 | CCTGTGTGAG AAAATCATAG CTGAGAGACA TTTTGATCAT |
| 401 | CTACGTGCAA AAAAAATACT CAGTAGAGAA GACACTGAAG |
| 441 | AAATTTCTTG TCGAACATCA AGTAGAAAAA GGGCTGGAAA |
| 481 | ATTGTTAGAC TACTTACAGG AAAACCCAAA AGGTCTGGAC |
| 521 | ACCCTTGTTG AATCTATTCG GCGAGAAAAA ACACAGAACT |
| 561 | TCCTGATACA GAAGATTACA GATGAAGTGC TGAAACTTAG |
| 601 | AAATATAAAA CTAGAACATC TGAAAGGACT AAAATGTAGC |
| 641 | AGTTGTGAAC CTTTTCCAGA TGGAGCCACG AACAACCTCT |
| 681 | CCAGATCAAA TTCAGATGAG AGTAATTTCT CTGAAAAACT |
| 721 | GAGGGCATCC ACTGTCATGT ACCATCCAGA AGGAGAATCC |
| 761 | AGCACGACGC CCTTTTTTTC TACTAATTCT TCTCTGAATT |
| 801 | TGCCTGTTCT AGAAGTAGGC AGAACTGAAA ATACCATCTT |
| 841 | CTCTTCAACT ACACTTCCCA GACCTGGGGA CCCAGGGGCT |
| 881 | CCTCCTTTGC CACCAGATCT ACAGTTAGAA GAAGAAGGAA |
| 921 | CTTGTGCAAA CTCTAGTGAG ATGTTTCTTC CCTTAAGATC |
| 961 | ACGTACTGTT TCACGACAAT GACACTTTAT TGCCTTTTAA |
| 1001 | TTTTTAATGA TGACAAAAAA TGTTTTAAAG AATATGACTT |
| 1041 | TTTATAAAAT GGCTGTAATC ATTTGTTTAC ATTTGATGCA |
| 1081 | TGTCTTTTAA AATGCAATGT AAGCATACTT TGTAAATAGG |
| 1121 | ATTTTTAGAA TTAAAAAAGC ATACTTCTAG GATAGCTAAC |
| 1161 | TGTAAATCAT GTTGATCATG TACTTTTTAG TAATTTCTTT |
| 1201 | TTTTCCTTTT TAAGGTCTTT CAGTACTTTT TTAAATATTT |
| 1241 | TCTATTTTAA GACTGATTTT AATAGGGAAT ATATCTCTAT |
| 1281 | TTGAGAATAG ACCCTTACTA GGAAGAACGT TTTTTCCTCA |
| 1321 | GTGCATTTGT GCTAGAAATT TTCAAGAGTC TAATAGTCTT |
| 1361 | TGCCAGTCAT TCAGCAGCAA ATTTTCAGCA TTAAGCTGTT |
| 1401 | CCTGTTCAGT AATAAAACCG GTCACTGATG GGAAAACTGC |
| 1441 | CAATATAGAA AAATAAAAAT CTCTTTTCCA CTCCATTGTC |
| 1481 | GTATAGGCAT GTAAACAGCC TCTTTTTGAT ACTGGAGGAA |
| 1521 | CACTTGATGG AGTGTGAGCC ACCTAAGATC TCGGTTTGCC |
| 1561 | AAAATTCATT TCTAATTAAC CTTACTAATT ATACTACTTT |
| 1601 | GTTAGGATTT TCACATTCTT GGCTTAATCA TTTTCATTCC |
| 1641 | TAAAGAAAAA TATCTTGGCC TAAACCTCAG TTATTACATG |
| 1681 | TAATTTGATG AGGTATTTTT TCCTTTTTTC TTTTTTTTTT |
| 1721 | TTTTTTTTTT TTGAGACAGT CTTGCTCTAT CGCCCAGGCT |
| 1761 | GGAGTGCAGT GGCGCATTCT AGGCTCACTG CAACTTCTGC |
| 1801 | CTCCCATGCT TACGTGATCC TCTCACCTCA GCCTCTCAAG |
| 1841 | TAATATAGCT GAGACTACAA GTGTGTGCCA CCATGCCTCA |
| 1881 | CTAATTTTTG TATTATTTTT GTAGAGACGG TGTTTTGCCA |
| 1921 | TGTTGGCCAG GCTGGTCTTG AACTCCTGGA CTCAAGCAAC |
| 1961 | CTACCCAGCG TGGCCTCCCA AAGTGCTGGG ATTACAGACA |
| 2001 | CGAGCCACCT CACCTAGCCT GATGAGATTT TTAAAAAATA |
| 2041 | TTTTCTCTGT ACTTTTCATT CTCTTTTAAT GAGGACCAAT |
| 2081 | GTACAGTTGA AATAACTGGA ACAAATTATT TTTGGTGTGT |
| 2121 | GTGACAATTC TGTTTTTAAT GCTATTTGAA CAAGTGGGCC |
| 2161 | ATTAGCCAGA TTTGTCTTTT TGTTGTAAAA CAAAATTTGA |
| 2201 | CTAATTTTAC ATGTTTATAA ATCTTATGCT CTCACTGTTT |
| 2241 | GTTTTTATTT AAATTACAAT TTTATCTGTT TCCTGACATT |
| 2281 | GTCTCCTATA TATTTCTATT ATTAATTGCA AAAACATAGA |
| 2321 | AATGGAAATT TTGCTATCAA CAATAAAATT TTTTTAAAGT |
| 2361 | AGTGAGTGCT ATTTTGGAGT TCCAAATTTT CAGTAGGAAG |
| 2401 | TATCTAAAAC TTTTTTTAAT ACGTGCCATT ATCTATAGAA |
| 2441 | AACATTACTT CAGGTTGTGA GATTGAGTTG CATTTCTGGA |
| 2481 | TGGACTGATG AATTTATCCG ACATGAAGAA GATTGGCATA |
| 2521 | TTAGCTTTAA AAATTTTTAA AGATTGGATT TTTTTTAGTA |
| 2561 | TAAGCACTTT CTAAGGATTA TAGAGAAATG TTTCACCTCC |
| 2601 | AATGCATAGC AAAAATAGTG GTGTTAGAAA GAAAATAGGT |
| 2641 | TACATTTAAG GAAGGTGCTT TAAAAAGCAG AAGCAGACTT |
| 2681 | TAAAATTAAA TTTGTGGACA CCTTTTTAAA AATTGAATCA |
| 2721 | AAGATTATAA TTTAGATATA CAATAACACC TATATATAGA |
| 2761 | TAAGTTTTAA CACTGAGTTT TCTTTCAGAC TGTTTTCTAA |
| 2801 | CTACATAGAC AATAAAATTA AGCTTTGCAT AAA |

Other isoforms of human BCL10 exist and have sequences with NCBI accession numbers NP_001307644.1 (GI: 1002639417), XP-011540699.1 (GI: 767906661), XP_011540700.1 (GI: 767906663), XP_011540701.1 (GI: 767906666).

Hence, various isoforms and variants of the human BCL10 proteins and nucleic acids can be present in populations of subjects. Any such isoforms and variants can also be detected and subjects with such isoforms can be treated using the methods described herein. Such isoforms and variants of the human BCL10 proteins and nucleic acids can have sequences with between 55-100% sequence identity to a reference sequence, for example to any of the human BCL10 sequences described herein. For example, the isoforms and variants of the human BCL10 protein and nucleic acids can have at least 55% sequence identity, at least 60%, at least 70%, at least 80%, at least 90%, at least 95%, at least 96%, at least 97% sequence, at least 98%, at least 99% identity to any of the sequences described herein. The sequence comparisons can be over a specified comparison window. Optimal alignment may be ascertained or conducted, for example, using the homology alignment algorithm of Needleman and Wunsch, J. Mol. Biol. 48:443-53 (1970).

However, as described herein, mutations at position 58 and truncation at or beyond position 135 of the BCL10 protein (e.g., positions 135-174, highlighted in SEQ ID NO:1) are correlated with aggressive forms of diffuse large B-cell lymphomas (DLBCLs), and such lymphomas can benefit more from treatment with MALT1 inhibitors than some other types of treatment. Accordingly subjects with mutations of their BCL10 protein at amino acid position equivalent to position 58 and/or a truncation at or beyond an amino acid position equivalent to position 135 of SEQ ID NO:1 can be diagnosed and treated even when those subjects have any BCL10 isoform or variant. In some cases the C-terminal BCL10 truncation can be with positions equivalent to amino acid positions 135 to 174 of SEQ ID NO:1.

BCL10 mutations can be detected in the genomes or mRNA samples from subjects by a variety of methods. Examples of methods that can be used include genomic sequencing, cDNA sequencing, mRNA sequencing, RFLP analysis, SNP analysis, PCR amplification, Northern blot analysis, Southern blot analysis, probe hybridization, or a combination thereof. Mutant BCL10 proteins can be detected in samples from subjects by a variety of methods, including antibody detection, PAGE analysis, Western blot analysis, BCL10 filament formation (or missing/aberrant filament formation), or combinations thereof.

Antibodies that specifically bind to BCL10 protein, and/or probes/primers that bind specifically to BCL10 DNA or BCL10 mRNA can be used to detect BCL10 mutations. Antibodies that bind to BCL10 protein are available, for example from Abcam, CellSignal, OriGene, Rockland, and ThermoFisher Scientific. Probes and primers that bind specifically to BCL10 mRNA can include segments of about 15-100 nucleotides that have at least 90% sequence identity or complementarity to a BCL10 coding region (e.g., to a BCL10 cDNA with the SEQ ID NO:2 sequence).

### Mucosa-Associated Lymphoid Tissue Lymphoma Translocation Protein 1 (MALT1) Inhibitors

A variety of MALT1 inhibitors can be used for treatment of subjects having BCL10 mutations at amino acid positions equivalent to position 58 of SEQ ID NO:1, and/or truncations of BCL10 at positions equivalent to 140 and beyond in the BCL10 C-terminus. The subjects can have diagnosed lymphoma and/or symptoms of lymphoma such as enlarged lymph nodes, night sweats, unusual weight loss, loss of appetite, tiredness/fatigue, fever, itchiness, or a combination thereof.

Examples of MALT1 inhibitors that can be used include MLT-748, JNJ-67690246, MI-2, Mepazine, MLT-943, JNJ-67856633, MLT-985, (R)-MALT1-IN-7, MLT-231, Z-VRPR-FMK (TFA), MLT-747, (S)-MALT1-IN-5, (R)-MALT I-IN-3, MALT1-IN-7, or combinations thereof. Such inhibitors can reduce tumor weight or tumor volume by at least 10%, or 20% or 30%, or 40%, or 50%, or 60%, or 75%, or 100% compared to an untreated control. In some cases, inhibitors can reduce tumor weight or tumor volume by at least 1.2-fold, or 1.5-fold, or 2-fold, or 3-fold, or 5-fold, or 7-fold, or 10-fold compared to an untreated control. Such a control can be a subject with untreated lymphoma.

MLT-748 is a reversible allosteric compound that binds MALT1 Trp580 side chain to lock the protease into an inactive form (Quancard et al., Nat Chem Biol 15:304-313 (2019)). A structure for MLT-748 is shown below.

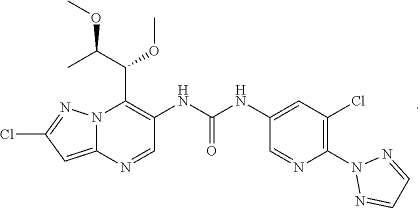 [SMILES: CO[C@H](c1c(NC(=O)Nc2cnc(-n3nccn3)c(Cl)c2)cnc2cc(Cl)nn12)[C@@H](C)OC]

JNJ-67690246 is an allosteric MALT1 inhibitor, having the structure shown below.

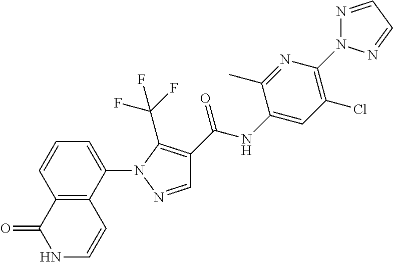 [SMILES: Cc1nc(-n2nccn2)c(Cl)cc1NC(=O)c1cnn(-c2cccc3c(=O)[nH]ccc23)c1C(F)(F)F]

MI-2 is an irreversible MALT1 inhibitor with the structure shown below.

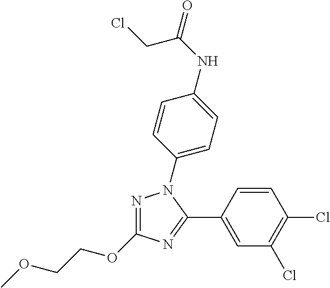 [SMILES: COCCOc1nc(-c2ccc(Cl)c(Cl)c2)n(-c2ccc(NC(=O)CCl)cc2)n1]

Mepazine (Pecazine) is a potent and selective MALT1 protease inhibitor with the structure shown below.

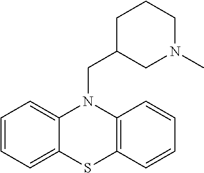 [SMILES: CN1CCCC(CN2c3ccccc3Sc3ccccc32)C1]

MLT-943 is a potent, selective and orally active MALT1 protease inhibitor with the structure shown below.

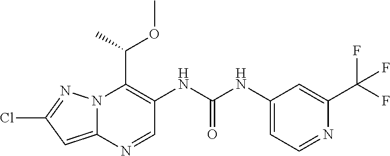 [SMILES: CO[C@@H](C)c1c(NC(=O)Nc2ccnc(C(F)(F)F)c2)cnc2cc(Cl)nn12]

JNJ-67856633 is an orally active, first-in-class, potent, selective and allosteric MALT1 protease inhibitor with the structure shown below.

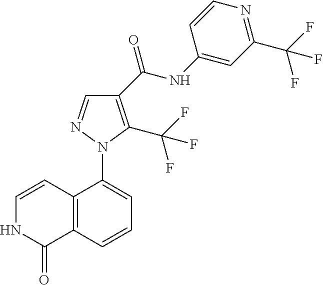 [SMILES: O=C(Nc1ccnc(C(F)(F)F)c1)c1cnn(-c2cccc3c(=O)[nH]ccc23)c1C(F)(F)F]

MLT-985 is a highly selective allosteric MALT1 inhibitor with the structure shown below.

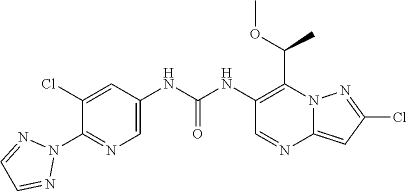 [SMILES: CO[C@@H](C)c1c(NC(=O)Nc2cnc(-n3nccn3)c(Cl)c2)cnc2cc(Cl)nn12]

(R)-MALT1-IN-7 (compound 142a) is a potent MALT1 protease inhibitor with the structure shown below.

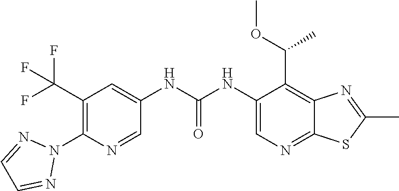 [SMILES: CO[C@H](C)c1c(NC(=O)Nc2cnc(-n3nccn3)c(C(F)(F)F)c2)cnc2sc(C)nc12]

(R)-MLT-985 (compound 11) is a potent MALT1 protease inhibitor with the structure shown below.

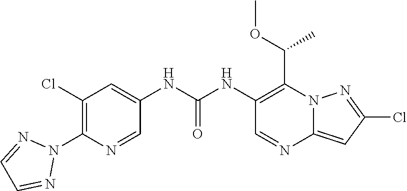 [SMILES: CO[C@H](C)c1c(NC(=O)Nc2cnc(-n3nccn3)c(Cl)c2)cnc2cc(Cl)nn12]

(R)-MALT1-IN-7 (compound 142a) is a potent MALT1 protease inhibitor with the structure shown below.

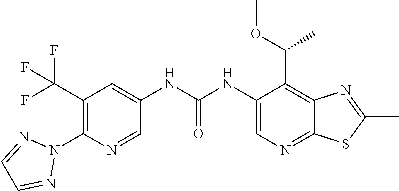 [SMILES: CO[C@H](C)c1c(NC(=O)Nc2cnc(-n3nccn3)c(C(F)(F)F)c2)cnc2sc(C)nc12]

(R)-MLT-985 (compound 11) is a potent MALT1 protease inhibitor with the structure shown below.

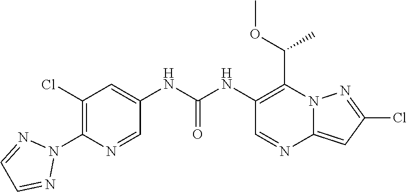 [SMILES: CO[C@H](C)c1c(NC(=O)Nc2cnc(-n3nccn3)c(Cl)c2)cnc2cc(Cl)nn12]

MLT-231 is a potent, highly selective allosteric MALT1 Inhibitor with the structure shown below.

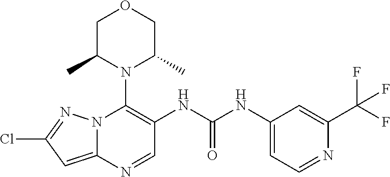 [SMILES: C[C@H]1COC[C@H](C)N1c1c(NC(=O)Nc2ccnc(C(F)(F)F)c2)cnc2cc(Cl)nn12]

Z-VRPR-FMK (TFA) (VRPR), a tetrapeptide, is a selective and irreversible MALT1 with the structure shown below.

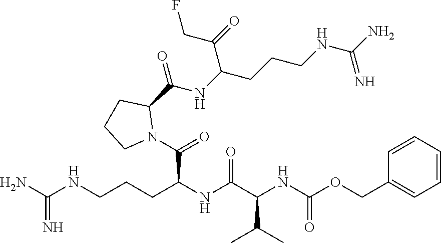 [SMILES: CC(C)[C@H](NC(=O)OCc1ccccc1)C(=O)N[C@@H](CCCNC(=N)N)C(=O)N1CCC[C@H]1C(=O)NC(CCCNC(=N)N)C(=O)CF]

MLT-747 is a potent, selective, allosteric inhibitor of MALT1, binds MALT1 in the allosteric Trp580 pocket, with the structure shown below.

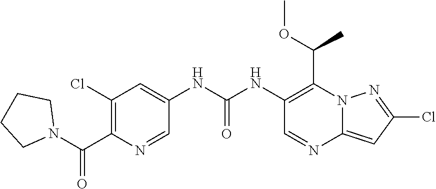 [SMILES: CO[C@@H](C)c1c(NC(=O)Nc2cnc(C(=O)N3CCCC3)c(Cl)c2)cnc2cc(Cl)nn12]

(S)-MALT1-IN-5 is a potent inhibitor of MALT1 protease, with the structure shown below.

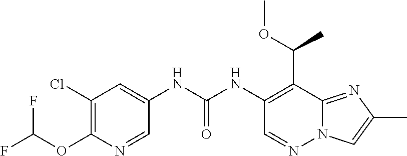 [SMILES: CO[C@@H](C)c1c(NC(=O)Nc2cnc(OC(F)F)c(Cl)c2)cnn2cc(C)nc12]

(R)-MALT1-IN-3 (compound 121) is a potent MALT1 protease inhibitor with the structure shown below.

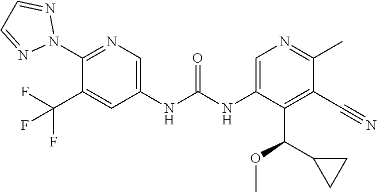 [SMILES: CO[C@@H](c1c(NC(=O)Nc2cnc(-n3nccn3)c(C(F)(F)F)c2)cnc(C)c1C#N)C1CC1]

MALT1-IN-7 (compound 142b) is a potent MALT1 protease inhibitor with the structure shown below.

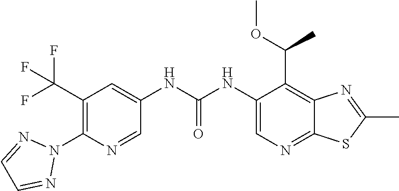 [SMILES: CO[C@@H](C)c1c(NC(=O)Nc2cnc(-n3nccn3)c(C(F)(F)F)c2)cnc2sc(C)nc12]

### Bruton's Tyrosine Kinase (BTK) Inhibitors

BTK is member of the Tec family that is important for the growth, differentiation and activation of myeloid-, mast- and B-cells. BTK is initially activated by membrane localization which is stimulated by the generation of PIP₃. As illustrated herein, subjects having BCL10 mutations at amino acid positions equivalent to position 58 of SEQ ID NO:1, and/or truncations of BCL10 at positions equivalent to 140 and beyond in the BCL10 C-terminus can benefit from treatment with a variety of MALT1 inhibitors. The subjects can have diagnosed lymphoma and/or symptoms of lymphoma such as enlarged lymph nodes, night sweats, unusual weight loss, loss of appetite, tiredness/fatigue, fever, itchiness, or a combination thereof.

However, some lymphoma patients may already have received one or more BTK inhibitors, and in some cases those patients are subjects who may be resistant to BTK inhibitors. Such subjects may be treated more effectively with MALT1 inhibitors.

Examples of BTK inhibitors that may have been used for various subjects include Ibrutinib, Zanubrutinib, Acalabrutinib, CGI 1746, LCB 03-0110, LFM-A13, PCI 29732, PF 06465469, (−)-Terreic acid, DD 03-171, or a combination thereof.

Ibrutinib is a potent and selective BTK inhibitor with the following structure:

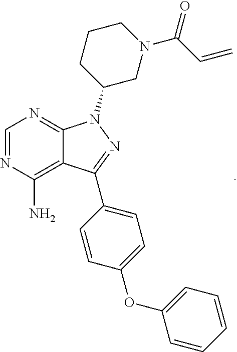 [SMILES: C=CC(=O)N1CCC[C@@H](n2nc(-c3ccc(Oc4ccccc4)cc3)c3c(N)ncnc32)C1]

Zanubrutinib is classified as a BTK) inhibitor with the following structure.

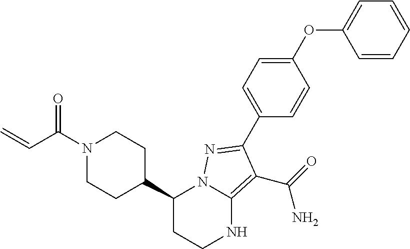 [SMILES: C=CC(=O)N1CCC([C@@H]2CCNc3c(C(N)=O)c(-c4ccc(Oc5ccccc5)cc4)nn32)CC1]

Acalabrutinib, sold under the brand name Calquence, is a BTK inhibitor with the following structure:

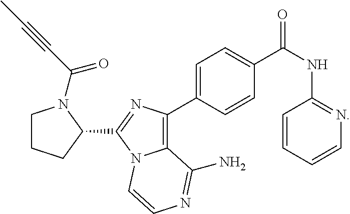 [SMILES: CC#CC(=O)N1CCC[C@H]1c1nc(-c2ccc(C(=O)Nc3ccccn3)cc2)c2c(N)nccn12]

CGI 1746 is a potent, reversible inhibitor of BTK with the following structure:

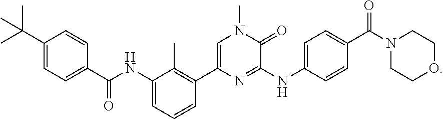 [SMILES: Cc1c(NC(=O)c2ccc(C(C)(C)C)cc2)cccc1-c1cn(C)c(=O)c(Nc2ccc(C(=O)N3CCOCC3)cc2)n1]

LCB 03-0110 salts are potent c-Src kinase inhibitors, as well as potent inhibitors of BTK. LCB 03-0110 has the following structure:

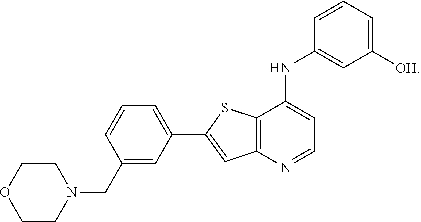 [SMILES: Oc1cccc(Nc2ccnc3cc(-c4cccc(CN5CCOCC5)c4)sc23)c1]

LFM-A13 is a potent and selective inhibitor of BTK, and has the following structure:

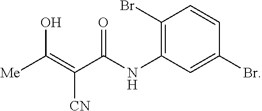 [SMILES: C/C(O)=C(\C#N)C(=O)Nc1cc(Br)ccc1Br]

PCI 29732 is a potent BTK inhibitor with the following structure:

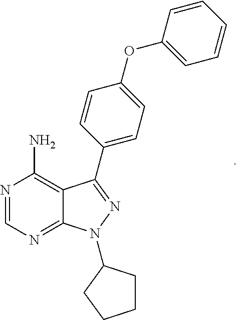 [SMILES: Nc1ncnc2c1c(-c1ccc(Oc3ccccc3)cc1)nn2C1CCCC1]

PF 06465469 is a potent inhibitor of interleukin-2 inducible T cell kinase (ITK) (IC₅₀=2 nM), and also exhibits inhibitory activity against BTK. PF 06465469 with the following structure:

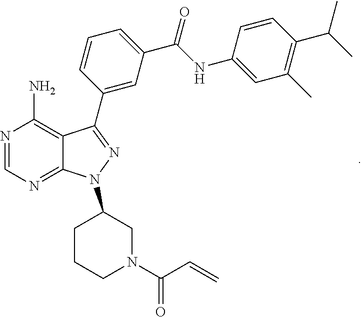 [SMILES: C=CC(=O)N1CCC[C@@H](n2nc(-c3cccc(C(=O)Nc4ccc(C(C)C)c(C)c4)c3)c3c(N)ncnc32)C1]

(−)-Terreic acid is a selective inhibitor of BTK with the following structure:

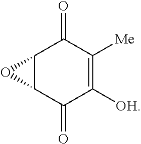 [SMILES: CC1=C(O)C(=O)[C@@H]2O[C@@H]2C1=O]

DD 03-171 is a potent and selective BTK Degrader (PROTAC®) with the following structure:

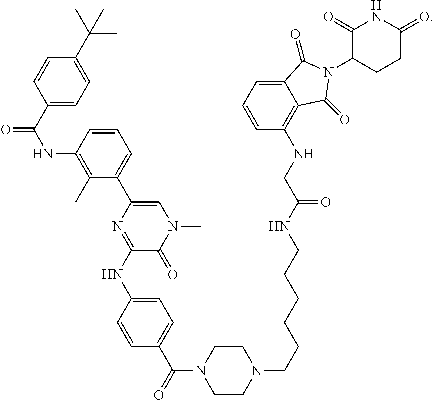 [SMILES: Cc1c(NC(=O)c2ccc(C(C)(C)C)cc2)cccc1-c1cn(C)c(=O)c(Nc2ccc(C(=O)N3CCN(CCCCCCNC(=O)CNc4cccc5c4C(=O)N(C4CCC(=O)NC4=O)C5=O)CC3)cc2)n1]

### Pharmaceutical Compositions

MALT1 inhibitors may be administered neat or, preferably, as pharmaceutical compositions. Pharmaceutical compositions of the invention include an appropriate amount of the MALT1 inhibitors in combination with an appropriate carrier as well as other useful ingredients.

MALT1 inhibitors can include the compounds described herein, and wherein applicable, acceptable salts thereof. Acceptable salts include, but are not limited to, those prepared from the following acids: alkyl, alkenyl, aryl, alkylaryl and alkenylaryl mono-, di- and tricarboxylic acids of 1 to 20 carbon atoms, optionally substituted by 1 to 4 hydroxyls; alkyl, alkenyl, aryl, alkylaryl and alkenylaryl mono-, di- and trisulfonic acids of 1 to 20 carbon atoms, optionally substituted by 1 to 4 hydroxyls; and mineral acids. Examples include hydrochloric: hydrobromic; sulphuric; nitric; phosphoric; maleic; acetic; salicyclic; p-toluenesulfonic; tartaric; citric; methanesulphonic; formic; malonic; succinic; naphthalene-2-sulphonic; and benzenesulphonic acid. Also, pharmaceutically-acceptable salts may be prepared as amine salts, ammonium salts, or alkaline metal or alkaline earth salts, such as sodium, potassium or calcium salts of the carboxylic acid group. These are formed from alkaline metal or alkaline earth metal bases or from amine compounds. In addition, analogs of the foregoing compounds that act as functional equivalents also are intended to be embraced as equivalents and within the scope of the invention.

Pharmaceutical compositions of MALT1 suitable for oral administration may be in the form of (1) discrete units such as capsules, cachets, tablets or lozenges each containing a predetermined amount of the MALT1; (2) a powder or granules; (3) a bolus, electuary or paste; (4) a solution or a suspension in an aqueous liquid or a non-aqueous liquid; or (4) an oil-in-water liquid emulsion or a water-in-oil liquid emulsion. Thus, compositions suitable for topical administration in the mouth, for example buccally or sublingually, include lozenges. Compositions suitable for parenteral administration include aqueous and non-aqueous sterile suspensions or injection solutions. Compositions suitable for rectal administration may be presented as a suppository.

Thus, pharmaceutical compositions of MALT1 inhibitors may be formulated using a solid or liquid carrier. The solid or liquid carrier would be compatible with the other ingredients of the formulation and not deleterious to the recipient. If the pharmaceutical composition is in tablet form, then MALT1 inhibitor(s) are mixed with a carrier having the necessary compression properties in suitable proportions and compacted in the shape and size desired. If the composition is in powder form, the carrier is a finely divided solid in admixture with the finely divided active ingredient. The powders and tablets may contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins. A solid carrier may include one or more substances that may act as flavoring agents, lubricants, solubilizers, suspending agents, fillers, glidants, compression aids, binders or tablet-disintegrating agents. A suitable carrier may also be an encapsulating material.

If the composition is a solution, suspension, emulsion, syrup, elixirs or pressurized compositions, then liquid carriers may be used. In this case, the MALT1 inhibitors are dissolved or suspended in a pharmaceutically acceptable liquid carrier. Suitable examples of liquid carriers for oral and parenteral administration include (1) water, (2) alcohols, e.g. monohydric alcohols and polyhydric alcohols such as glycols, and their derivatives, and (3) oils, e.g. fractionated coconut oil and arachis oil. For parenteral administration, the carrier may also be an oily ester such as ethyl oleate and isopropyl myristate. Liquid carriers for pressurized compositions include halogenated hydrocarbon or other pharmaceutically acceptable propellent. The liquid carrier may contain other suitable pharmaceutical additives such as solubilizers; emulsifiers; buffers; preservatives; sweeteners; flavoring agents; suspending agents; thickening agents; colors; viscosity regulators; stabilizers; osmo-regulators; cellulose derivatives such as sodium carboxymethyl cellulose; anti-oxidants; and bacteriostats. Other carriers include those used for formulating lozenges such as sucrose, acacia, tragacanth, gelatin and glycerin as well as those used in formulating suppositories such as cocoa butter or polyethylene glycol.

If the composition is to be administered intravenously or intraperitoneally by infusion or injection, solutions of the MALT1 inhibitors may be prepared in a solvent (e.g. water), optionally mixed with a nontoxic surfactant. Dispersions can also be prepared in water, glycerol, liquid polyethylene glycols, triacetin, oils, and mixtures thereof. Under ordinary conditions of storage and use, these preparations contain a preservative to prevent the growth of microorganisms. The composition suitable for injection or infusion may include sterile aqueous solutions or dispersions or sterile powders comprising the active ingredient, which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium as described above. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin. Sterile injectable solutions are prepared by incorporating the MALT1 inhibitors in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the MALT1 inhibitors, plus any additional desired ingredient present in the previously sterile-filtered solutions.

Pharmaceutical compositions of the invention may be in unit-dose or multi-dose form or in a form that allows for slow or controlled release of the MALT1 inhibitors. Each unit-dose may be in the form of a tablet, capsule or packaged composition such as, for example, a packeted powder, vial, ampoule, prefilled syringe or sachet containing liquids. The unit-dose form also may be the appropriate number of any such compositions in package form. Pharmaceutical compositions in multi-dose form may be in packaged in containers such as sealed ampoules and vials. In this case, the MALT1 inhibitors may be stored in a freeze-dried (lyophilized) condition requiring only the addition of a sterile liquid carrier immediately prior to use. In addition, extemporaneous injection solutions and suspensions may be prepared from sterile powders, granules and tablets of the kind previously described.

In general, dosage forms of the invention comprise an amount of at least one of the MALT1 inhibitors effective to treat or prevent the clinical symptoms of a disease (e.g. a lymphoma). Any statistically significant attenuation of one or more symptoms of a lymphoma is considered to be a treatment thereof.

The absolute weight of a given MALT1, or other therapeutic agent that is included in a unit dose can vary widely. For example, about 0.01 to about 2 g, or about 0.1 to about 500 mg, of at least one MALT1, or other therapeutic agent can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of a MALT1, or other therapeutic agent(s) can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

### Definitions

The term “about” as used herein when referring to a measurable value such as an amount, a length, and the like, is meant to encompass variations of ±20% or ±10%, more preferably ±5%, even more preferably ±1%, and still more preferably ±0.1% from the specified value.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

A “coding region” or a sequence which “encodes” a selected polypeptide or a selected RNA, is a nucleic acid molecule which is transcribed (in the case of DNA templates) into RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, ncRNAs, tracrRNAs, ncRNAs modified to include heterologous sequences, cDNA from viral, prokaryotic or eukaryotic ncRNA, mRNA, viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding region is capable of effecting the expression of the encoded sequence when the proper polymerases are present. The promoter need not be contiguous with the coding region, so long as it functions to direct the expression thereof. Thus, for example, intervening untranslated yet transcribed sequences can be present between the promoter sequence and the coding region and the promoter sequence can still be considered “operably linked” to the coding region.

“Encoded by” refers to a nucleic acid sequence that codes for a polypeptide or RNA. For example, a polypeptide sequence or a portion thereof is encoded by the nucleic acid sequence. The RNA sequence or a portion thereof contains a nucleotide sequence that is encoded by a DNA (or other nucleic acid) sequence.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein, DNA, or RNA or cause other adverse consequences. That is, a nucleic acid or peptide can be purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are available and include, for example, ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Mammalian cell” refers to any cell derived from a mammalian subject. The mammalian cells can in some cases be suitable for transfection with vector systems. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats: farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals. In some cases, the subject is a human.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “donor polynucleotide” or “donor DNA” refers to a nucleic acid or polynucleotide that provides a nucleotide sequence of an intended edit to be integrated into the genome at a target locus by HDR or recombineering.

“Administering” a nucleic acid, such as an expression cassette, comprises transducing, transfecting, electroporating, translocating, fusing, phagocytosing, shooting or ballistic methods, etc., i.e., any means by which a nucleic acid can be transported across a cell membrane.

The subject matter disclosed herein is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

The invention will be further described by the following non-limiting examples.

### Example 1: Materials and Methods

This Example describes some of the materials and methods used in the development of the invention.

### Cell Culture

Raji, HBL1, TMD8 and RIVA were cultured in RPMI supplemented with 10% FBS, 2 mM L-glutamine and 10 mM HEPES. OCI-Ly10 was cultured in Iscove's medium supplemented with 20% FBS, 2 mM L-glutamine. 293T was cultured in Dulbecco's Modified Eagle Medium with 10% FBS. All cell lines were authenticated by University of Arizona Genetic Core, grown in presence of 1% penicillin G and streptomycin and at 37° C. in a humidified atmosphere of 5% CO₂. HBL1 and RIVA was obtained from Jose A. Martinez-Climent (Universidad de Navarra, Pamplona, Spain); TMD8 was obtained from Louis M. Staudt (National Cancer Institute, Bethesda, Maryland, USA); OCI-Ly10 cell lines were obtained from the Ontario Cancer Institute (OCI).

### Virus Production and Transduction

Lentiviruses were produced in 293T cells by co-transfecting shRNA or overexpression vectors with packaging vectors psPax2 (Addgene #12260, RRID: Addgene_12260) and psMD2.g (Addgene #12259, RRID: Addgene_12259) at the 4:3:1 ratio in serum free media. The sequences of the short hairpin sequences used were:

shNonTargeting:
(SEQ ID NO: 4)
CAACAAGATGAAGAGCACCAA;
shCARD11#1:
(SEQ ID NO: 5)
GGACGACAACTACAACTTAGC;
and
shCARD11#3:
(SEQ ID NO: 6)
TGGTCAAGAAGCTGACGATTC.

The supernatant containing virus particles were harvested 48 h and 72 h after transfection, filtered through 0.45 μm filter and then concentrated with PEG-it according to manufacturer's instructions (LV825A-1, System Biosciences). Virus was resuspended with PBS containing 25 μM HEPES and added to cells for overnight infection. Cells were selected 24 h post transfection by adding puromycin (Sigma), blasticidin (Invivogen) or G418 (Life Technologies) for at least 48 h.

### Xenograft

All mice experiments were approved by Institutional Animal Care & Use Committee (IACUC) at Weill Cornell Medicine and were performed following the IACUC guidelines. Eight to ten weeks of female NOD.Cg-prkdc^{scid} Il2rg^{tm1Wj1}/SzJ (NSG, RRID: IMSR_JAX: 005557) mice were obtained from The Research Animal Resource Center (RARC) at Weill Cornell Medicine. 5×10⁶ HBL1 or 10⁷ million OCI-Ly10 and their derived engineered cells were resuspended with PBS/Matrigel (1:1) and subcutaneously injected to the right flank of mice. Treatments were started when tumor volume reached an average of 100 mm³. Ibrutinib was prepared in corn oil with 10% (v/v) DMSO or 0.5% methylcellulose in water and administrated p.o. with 25 or 37.5 mg/kg once per day. JNJ-67690246 was prepared in PEG400 with 10% (w/v) PVPVA64 and administrated p.o. with 100 mg/kg twice per day. Tumor volume was monitored 2˜3 times/week with digital caliper and calculated using the following formula. smallest diameter²×largest diameter×0.5.

### Growth Inhibition Assay

DLBCL cell lines were cultured in exponential condition and the cell growth was determined by CellTiter Glo (Promega). 3000-5000 cells were seeded and cultured in each well of 384 well plate for 96 h and treated with compounds every 48 h. Luminescence was read at the endpoint with the Synergy NEO microplate reader (BioTek). The value of compound treated cells was normalized to their vehicle treated controls and then used to calculate IC50 in GraphPad Prism (RRID:SCR_002798).

### NF-κB Reporter Assay

For NF-κB reporter assay in 293T, the plasmids expressing different BCL10 mutations were transiently transfected into 293T cells using Lipofectamine (Invitrogen). Renilla luciferase plasmid was co-transfected as an internal control. 24 h after transfection, cells were collected and luciferase activities were measured in Synergy NEO microplate reader (BioTek) with the Dual Luciferase Reporter Assay System (Promega) according to the manufacturer's instructions and normalized to Renilla luciferase activity. To generate stable NF-κB reporter cells, the lentivirus expressing 3×NF-κB response element followed by a luciferase firefly was made and infect the parental cells. Puromycin was then added for antibiotic selection 24 h after infection. Reporter cells were further validated by BTK inhibitor and MALT1 protease inhibitor treatment and PMA/IO stimulation. NF-κB reporter cells expressing BCL10 were generated by infecting reporter cells with different lentiviral BCL10 isoforms (co-expressing GFP), followed by sorting out GFP+ cells. Stable NF-κB reporter cells were harvested at the indicated conditions and lysed with 1× passive lysis buffer at room temperature for 20 min. The lysate was briefly centrifuged and the supernatant was collected for luciferase activity. All the assays were presented as mean±SEM of three independent experiments.

### MALT1 GloSensor Assay

The generation of Raji MALT1 GloSensor reporter cell has been previously described⁴⁴. All other GloSensor reporter cells were generated by infecting parental cells with lentiviral MALT1-GloSensor (pLex306 backbone), followed by antibiotic (blasticidin) selection. All derived GloSensor cells were further validated by MALT1 protease inhibitor treatment and PMA/IO stimulation.

### Immunoprecipitation

Lymphoma cells (10⁸) were collected, washed with cold PBS and resuspended with lysis buffer (1% NP40, 10% glycerol, 150 mM NaCl, 20 mM Tris-HCL pH 7.5, and freshly added protease inhibitors). The lysates were centrifuged at 15,000 g, 4° C. for 15 min and the supernatant was then collected and incubated with 50 μL equilibrated anti-Flag magnetic beads (Sigma-Aldrich Cat #M8823, RRID:AB_2637089) at 4° C. for 3 h. The beads were washed 3 times with lysis buffer and followed by 3 times washing with the lysis buffer without NP40. SDS loading buffer without non-reducing reagent was added and boiled at 95° C. for 5 min. The elution was added for 0-mercaptoethanol (final 10%), and ready to run western blot after boiling at 95° C. for 5 min.

### Western Blotting

Whole cell lysates extracted with RIPA buffer or IP elution were separated by SDS-PAGE gels and followed by transferring to PVDF membranes. Membranes were incubated with indicated primary antibodies: anti-Flag (Sigma-Aldrich Cat #F3165, RRID:AB_259529), anti-MALT1 (Santa Cruz Biotechnology Cat #sc-46677, RRID:AB_627909), anti-CARD11 (Abcam Cat #ab113409, RRID:AB_10861854), anti-O-Actin (AC-15, Sigma-Aldrich), and then mouse/rabbit peroxidase-conjugated secondary antibodies (Cell Signaling Technology). Protein intensity was detected with enhanced chemiluminescence using ChemiDoc imaging system (Bio Rad).

### Driver Mutation Analysis

The Driver mutation analysis is performed using Fishhook (see website at github.com/mskilab/fishHook) on a total of 243 ABC-DLBCL cases from NCI cohort. Fishhook is a model built with mutational calls, a set of hypothesis intervals, eligible genomic ranges and a set of genomic covariates that identifies the depletion and enrichment of genomic interval statistically. The model used a gamma-Poisson regression to implement the maximum likelihood approximation with consideration of user assigned covariates and expected mutation density to the hypothesis. With this approach, the model helps us to identify enriched mutations with consideration like chromatin features, sequence context composition and gene expression.

Eligible region is defined using genecode v19 and fractional coverage of hg19 positions provided by Agilent exome coverage. The model was also fed covariates that defines B cell specific transcriptional states and chromatin state information for the model. The covariates of ABC specific transcriptional states are generated by number of the overlap between the TSS site of genes TPM >2 in the half the ABC-DLBCL cases from the same NCI cohort within 10 kb the eligible regions. The covariates of B-cell specific chromatin states are generated by number of the overlap between H3K27Ac Peaks that previously reported in the B cell within 100 kb of the eligible regions and the ATAC peaks of the B cells within 10 kb of the eligible. A total of 3 covariates was fed to the fishhook model. Here we noted genes of FDR <0.05 and BCL10 have an FDR of 1.4e⁻¹⁰. QQ-plot is plotted by pairing observed −log 10 transformed quantiles of observed P values (y-axis) with their corresponding −log 10 transformed quantiles from the uniform distribution (x-axis).

### Biochemical Evaluation of MALT1 Protease Activity

MALT1 protease activity was assessed in vitro using full-length MALT1 protein (Strep-MALT1(1-824)-His) purified from baculovirus-infected insect cells. The tetrapeptide LRSR is coupled to 7-amino-4-methylcoumarin (AMC) and provides a quenched, fluorescent substrate for the MALT1 protease (SM Biochemicals). Cleavage of AMC from the arginine residue results in an increase in coumarin fluorescence measured at 460 nm (excitation 355 nm). Diluted compounds were pre-incubated with MALT1 enzyme for 50 minutes at room temperature (RT). Substrate was added and the reaction was then incubated for 4 h at RT, after which fluorescence was measured.

### IL-6/10 Secretion Assay Using DLBCL Cell Line

Secretion of the IL-6 and IL-10 cytokines by OCI-Ly3 ABC-DLBCL cells was measured using a Mesoscale assay (MSD). MALT1 inhibition results in a decrease of IL-6/10 secretion. OCI-Ly3 cells were treated with diluted compounds for 24 h at 37° C. and 5% CO2. After 24 h of incubation, 50 μL of the supernatant was transferred to an MSD plate (V-Plex Proinflammation Panel 1 [human] kit) and incubated for 2 h at RT followed by a 2 h incubation with IL-6/10 antibody solution. Plates were read on a SECTOR imager.

### Protein Expression and Purification

all Constructs of BCL10, MALT1 are from Human Sequences. Full-Length WT and mutant BCL10 constructs with N-terminal MBP tag were generated in vector pDB-His-MBP with a 3C protease site between MBP and BCL10. Full length His-tagged MALT1 cloned into pET29b was purchased from Addgene (RRID:Addgene_48968) and was expressed in E. coli.

All proteins were purified by either Ni-NTA resin (Qiagen) or Amylose resin followed by gel filtration chromatography (Superdex 200 10/300 GL, GE Healthcare). BCL10 FL and mutant filaments were purified by MBP affinity column in binding buffer containing 25 mM Tris at pH 7.5, 300 mM NaCl, 1 mM TCEP, followed by Superdex 200 gel filtration chromatography in buffer containing 20 mM Tris at pH 7.5, 150 mM NaCl and 1 mM TCEP, resulted in isolation of a monomeric fraction of BCL10. Then monomeric MBP-BCL10 was cleaved by 3C protease and incubated at RT for 2 hours in order to allow filaments formation. This step was followed by another Superdex 200 gel filtration chromatography and BCL10 filaments were isolated at the void peak for structure determination and thermostability assay. Isolation of BCL10 (1-140)-MALT1 complex was performed in a similar way in which BCL10 and MALT1 were purified separately. BCL10 pre-formed filaments after 3C cleavage were added mixed together with FL MALT1 to form a complex.

### Negative Stained Electron Microscopy

Copper grids coated with layers of plastic and thin carbon film were glow-charged before 5 μl of purified complexes were applied. Samples were left on the grids for 1 minute followed by negative staining with 1% uranyl formate for 30 seconds and air dried. In vitro BCL10 WT and mutants, BCL10/MALT1 and CBM were imaged with JEOL 1200EX or Tecnai G² Spirit BioTWIN at Harvard Medical School EM facility operating at 80 keV.

### Cryo-Electron Microscopy (Cryo-EM) Data Collection

Cryo grids for BCL10 R58Q and BCL10 E140X/MALT1 filaments were prepared by applying 3 μl of protein sample on a c-flat (1.2/1.3) 300 mesh grids. Grids were plunged by using vitrobot (FEI) at 4° c. with 3 sec blotting and force 4. For BCL10 R58Q data collection, 3439 movies were collected at super resolution mode with using Arctica microscope at UMASS facility, operated at 200 kv facility with k2 camera. The movies were collected automatically using SerialEM data collection at a nominal magnification of 36,000 and a pixel size of 0.435 Å, with a total dose of 38 e/A2 which was fractionated into 40 movie frames, with defocus range of −1-2.5 μm. For BCL10 E140X-MALT1 collection, 700 movies were collected at super resolution mode with using 300 kV FEI Titan Krios microscope equipped with FEI Falcon II detector at PNCC cryo-EM facility, operated at 300 kv with Falcon3 camera. The movies were collected automatically using SerialEM data collection at a nominal magnification of 47,000 and a pixel size of 0.4 Å, with a total dose of 55 e/Å2 which was fractionated into movie frames.

### Cryo Electron Data Processing

For helical reconstruction of BCL10 R58Q and BCL10 E140X/MALT1, Motioncor2 was used for drift correction and Micrographs were CTF corrected by using CTFFIND4. Data was processed with using Relion (3.1). The resolutions of the reconstruction were determined by FSC to 4.6 Å and 4.3 Å, respectively. Model building was performed in program Coot36. Refinement was performed against the using Phenix refine50 Structural presentations were generated using Pymol (DeLano Scientific) and Chimera (Pettersen et al. J Comput Chem 25:1605-1612 (2004).

### Confocal Imaging

Time lapsed movies of full length labeled Alexa488-BCL10 FL, BCL10 R58Q and BCL10 E140X were recorded with using Nikon spinning disk confocal microscope at Harvard Micron facility for periods of 30 min-1 hr with 1 minute interval, with using ×100 objective. 3C was added at a sub-molar ratio to allow MBP cleavage to occur within 2-3 minutes in order to provide ample time for setting up the microscope and starting the recording.

For concentration determination, labeled Alexa488-BCL10 FL, R58Q and E140X filaments were formed at increasing concentrations ranging between 0.1 μM-1 μM. 4 h after cleavage with 3C and incubation at RT, samples were placed on a 35 mm bottom glass dish and imaged with using spinning disk confocal microscope with using ×100 objective.

### Fluorescence Quenching Assay

Purified full length MBP-BCL10, BCL10 R58Q and BCL10 E140X were mixed with 5-fold molar excess of Alexa-488-C5-maleimide (Invitrogen) and incubated at 4° C. temperature for O/N. Gel filtration chromatography (Superdex 200, GE Healthcare) was used to remove free dyes. Fluorescence polarization assay was performed at 18° C. in buffer containing 20 mM Tris at pH 7.5, 150 mM NaCl, and 0.5 mM TCEP and in 20 p0 volume. 3 μM of labeled MBP-BCL10 were cleaved with 3C in the presence of increasing amount of MALT1. The fluorescence quenching was measured right after 3C addition for 2 h by using NEO plate reader (Biotek) using excitation/emission wavelengths of 495 nm/519 nm.

### Protein Stability

Purified BCL10 FL, R58Q and E140X filaments purified from the void peak of Superdex200 were mixed with 1-fold protein thermal shift dye (Thermo Fisher Scientific). Thermal scanning (25 to 95° C. at 1° C./min) was performed and melting curves were recorded on a StepOne RT-PCR machine. Data analysis was done by Protein Thermal Shift™ Software (Thermo Fisher Scientific).

### Immunofluorescence

HBL1 cells cultured in fresh media were mixed in 1:1 ratio with cytospin. Cells were spined at 800×g for 5 min. Cell pellets were resuspended with cytospin and plated on CELLview 4-compartment dishes (Greiner Bio-One). Cells were left at RT overnight and were fixed with 100% cold methanol for 5 minutes at −20° C., followed by cell permeabilization with 0.1% Triton X-100 in PBS-Tween (PBST) for 10 minutes. Cells were incubated with blocking buffer containing 3% BSA for 3 h, in order to minimize non-specific binding. After blocking, Cells were incubated overnight at 4° C. with FLAG primary antibody (Sigma-Aldrich Cat #F1804, RRID:AB_262044). After incubation, cells were washed with PBST 3 times and incubated with AlexaFluor488-conjugated anti-mouse IgG (Abcam Cat #ab1l50113, RRID:AB_2576208) for 1 h at room temperature. After incubation, cells were washed with PBS and then stained with Hoechst for 10 minutes (1:500, Immunochemistry Technologies, Cat #639). Cells were imaged using spinning disk confocal microscope with using ×100 objective.

### IHC Staining of p65

Formalin-fixed paraffin-embedded (FFPE) tissue sections of 4 μm thickness were cut from a tissue microarray composed of duplicate 0.6 mm cores from 298 cases of de novo DLBCL. Slides were processed using standard immunohistochemistry protocols and stained with an antibody against NF-κB p65 (Cell Signaling Technology Cat #8242, RRID:AB_10859369, 1:500 dilution). Appropriate staining was verified in sections of benign tonsil, heart and liver. Stained slides were assessed by an expert hematopathologist for nuclear expression of p65 in DLBCL tumor cells, scored as a percentage of tumor cell nuclei.

### Example 2: BCL10 Mutations are Genetic Drivers and Occur in Two Broad Classes

As a first approach to explore structure-function of BCL10 mutations, the inventors merged and evaluated DLBCL sequencing databases (Chapuy et al. Nat Med 24:679-690 (2018); Schmitz et al., N Engl J Med 378:1396-1407 (2018); Lacy et al., Blood 135: 1759-1771 (2020); Karube et al., Leukemia 32:675-684 (2018); Morin et al. Blood 122:1256-1265 (2013) (n=2255). Seventy-five (75) BCL10 mutant patients were identified.

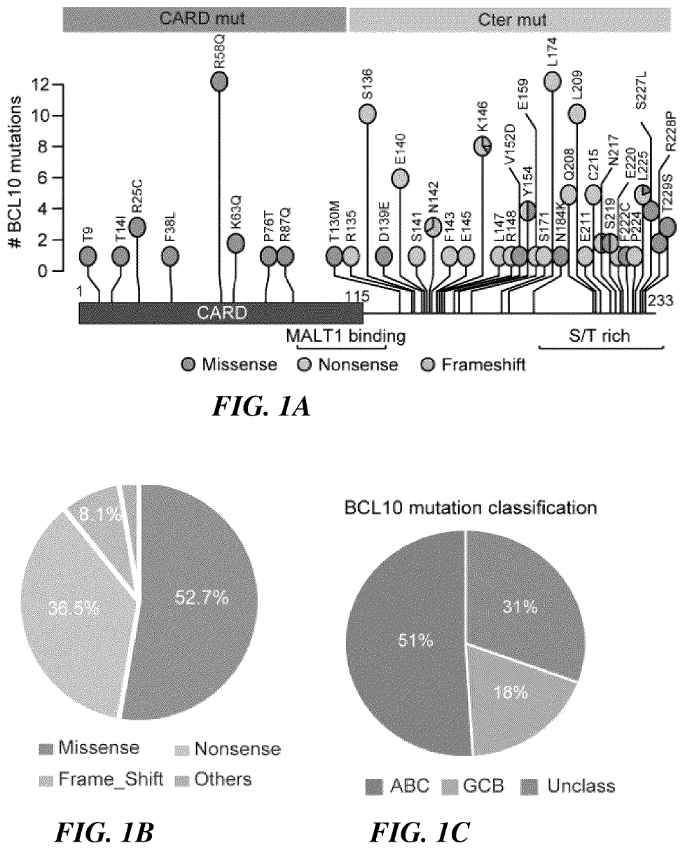

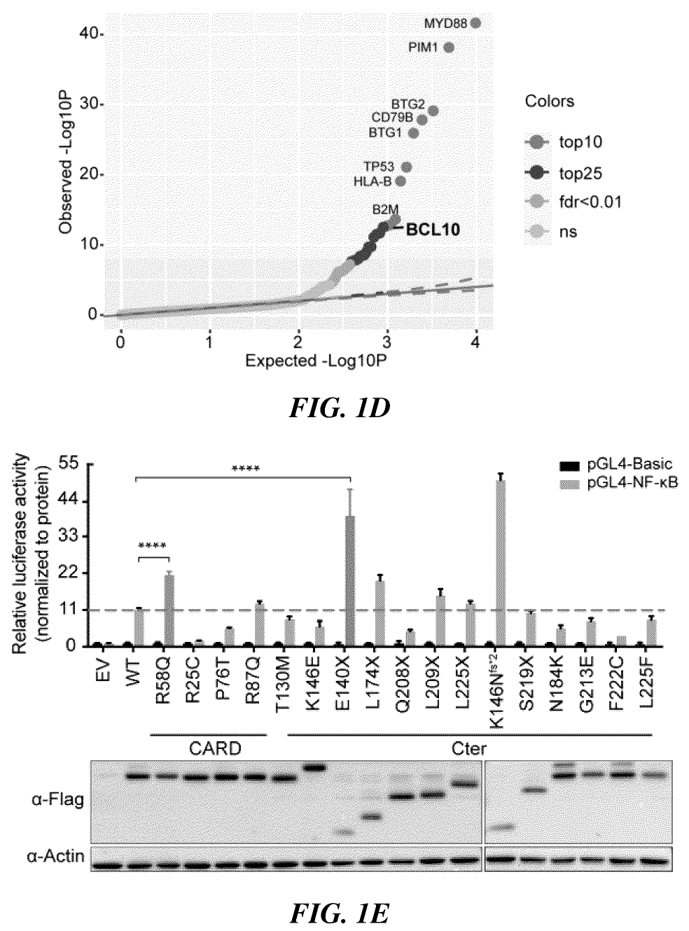

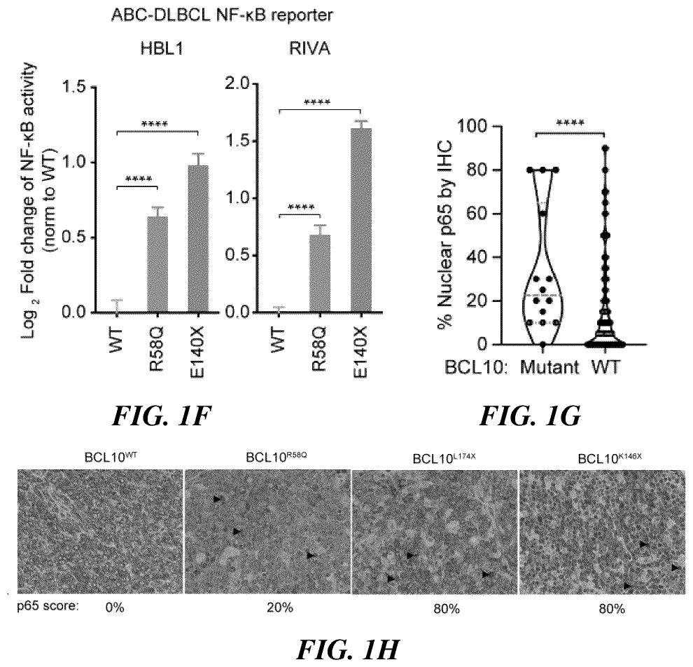

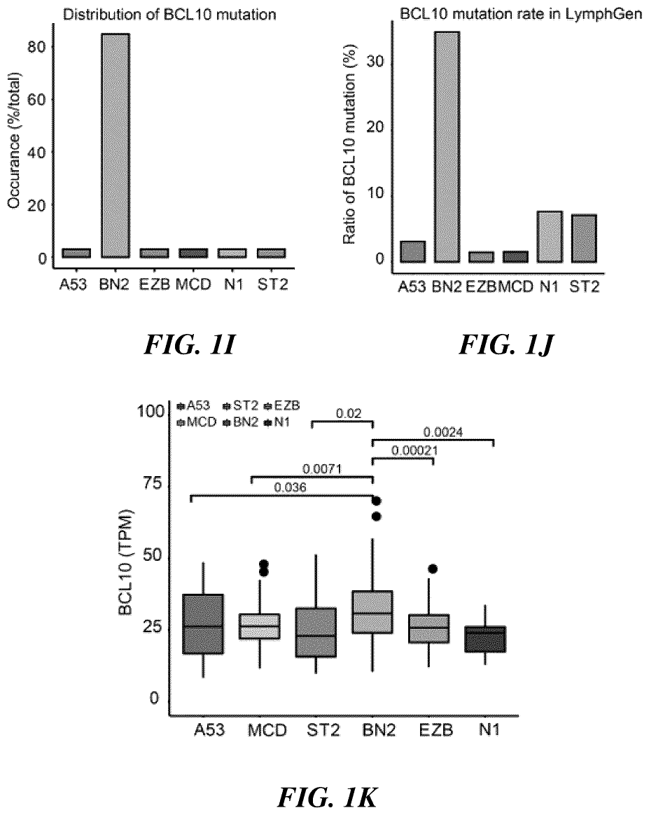

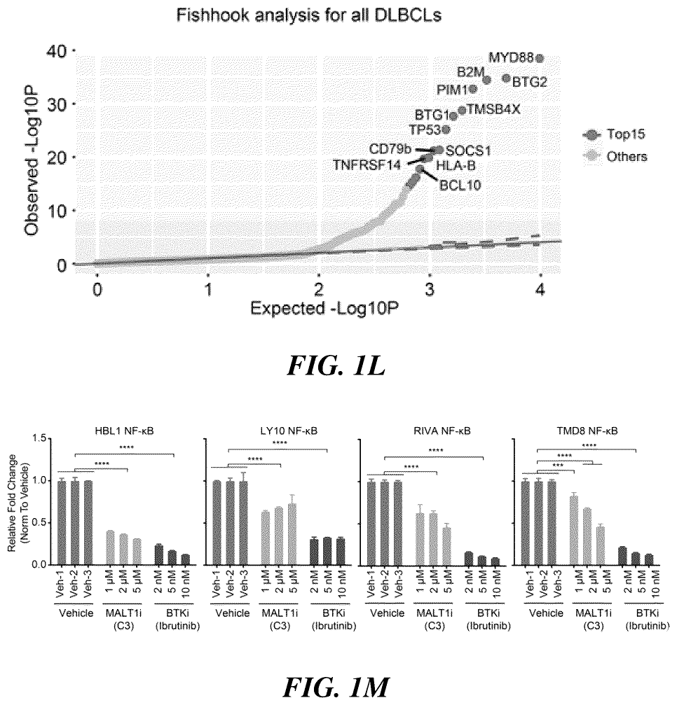

BCL10 contains a structured caspase activation and recruitment domain (CARD) at its N-terminal half that mediates interaction with other CARD proteins and polymerization of BCL10 into fibrils. The C-terminal region of BCL10 is unstructured and contains serine and threonine residues targeted for post-translational modifications (Qiao et al., Mol Cell 51:766-779 (2013); Thys et al. Front Oncol 8:498 (2018)). BCL10 mutations affected both regions. Although mutations in the CARD were all missense mutations with a prominent hotspot at Arginine 58, in contrast a majority of those in the C-terminal region were truncating (nonsense or frameshift) mutations with a number of hotspot residues observed (FIG. 1A-1B).

The BCL10 mutations in a cohort of patients were examined with rigorous cell of origin and genetic cluster information (Schmitz et al. N Engl J Med 378:1396-1407 (2018); Alizadeh et al., Nature 403.503-511 (2000)). The inventors discovered that 51% of the mutations occurred in ABC-DLBCLs, 31% of the mutations were unclassifiable cases, and 18% of mutations occurred in GCB-DLBCLs (FIG. 1C). However, the incidence of BCL10 mutation was highest in the unclassifiable patients, followed by the ABC-DLBCLs. Using the LymphGen classification system, most BCL10 mutations were observed in the BN2 class of DLBCLs (FIG. 1I), which had the highest incidence of these lesions (35% of patients, FIG. 1J). BCL10 expression was also highest among the BN2 cases (FIG. 1K).

To determine whether BCL10 somatic mutations were likely to be robust genetic drivers of ABC-DLBCLs, we performed a rigorous genomic co-variate “Fish-hook” analysis³⁶ controlling for gene size, as well as GC B-cell gene expression profiles, activating promoter histone marks, chromatin accessibility profiles and others. This analysis captured BCL10 as one of the top 10 driver mutations in ABC-DLBCL, along with genes such as MYD88, CD79B, PIM1 and TP53 (FDR<0.01, FIG. 1D). BCL10 mutations were still among the top 15 drivers when considering all DLBCLs (FIG. 1L).

To survey whether the different classes of BCL10 mutations had a functional impact on NF-κB signaling, a panel of CARD and C-terminal mutants were expressed, as well as wild-type BCL10, together with an NF-κB luciferase reporter in 293T cells. The wild type BCL10 was able to induce NF-κB activity (FIG. 1E). Most of the CARD and C-terminal mutations also induced NF-κB activity. However, the hotspot missense mutant BCL10^{R58Q} showed significantly higher NF-κB activity compared to wildtype BCL10, as did the truncating mutants at position 140 (BCL10^{E140X}) and position 146 (BCL10^{K146Nfs*2}) (FIG. 1E). When considering representative CARD and C-terminal mutations, markedly increased NF-κB reporter induction occurred in ABC-DLBCL cells that already exhibited chronic BCR activation including cell types MCD (HBL1) and BN2-DLBCL (RIVA) (FIG. 1F), also validating that this reporter reacts as expected to disruption of NF-κB signaling (FIG. 1M). Hence, both missense and truncating BCL10 mutations yield a significant gain of function effect on NF-κB activation. When an additional MCD cell line, TMD8, was evaluated a reduction in IκBα abundance was observed in cells expressing BCL10^{E140X} truncating mutant (FIG. 1N).

To determine if such findings could be validated in primary human DLBCLs, immunohistochemistry staining of p65 was performed in a set of tissue microarrays containing biopsies from 298 genetically annotated DLBCL patients. We found that BCL10 mutant DLBCLs manifested significantly increased p65 nuclear staining scores compared to BCL10 WT cases (Mann-Whitney p<0.0001, FIG. 1G). BCL10 truncating mutations associated with the highest nuclear p65 scores, whereas BCL10^{R58Q} CARD mutants less abundance of nuclear p65 that was still higher than in BCL10 WT cases (FIG. 1H).

### Example 3: BCL10 Polymerization is Greatly and Moderately Enhanced Respectively by E140X and R58Q Mutants

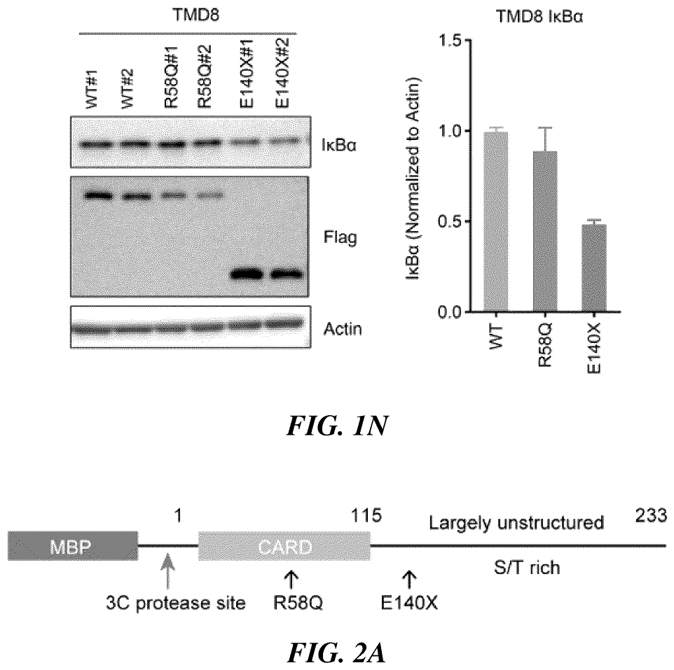

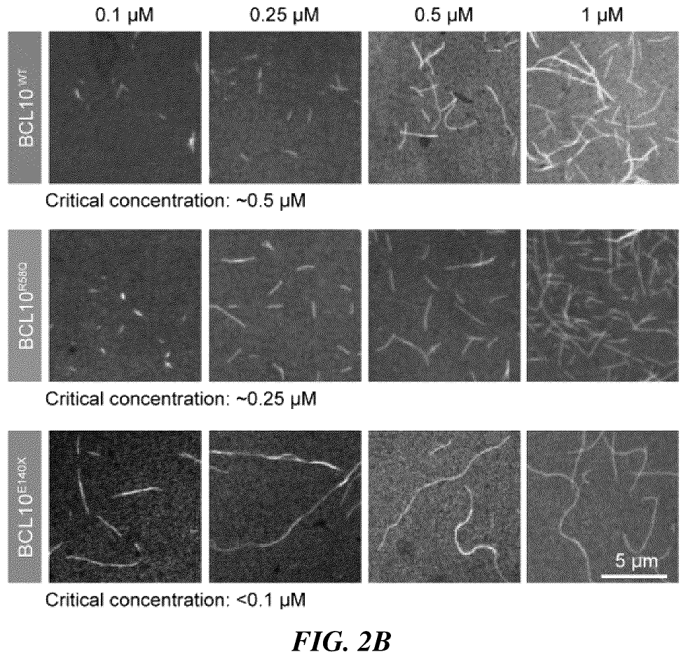

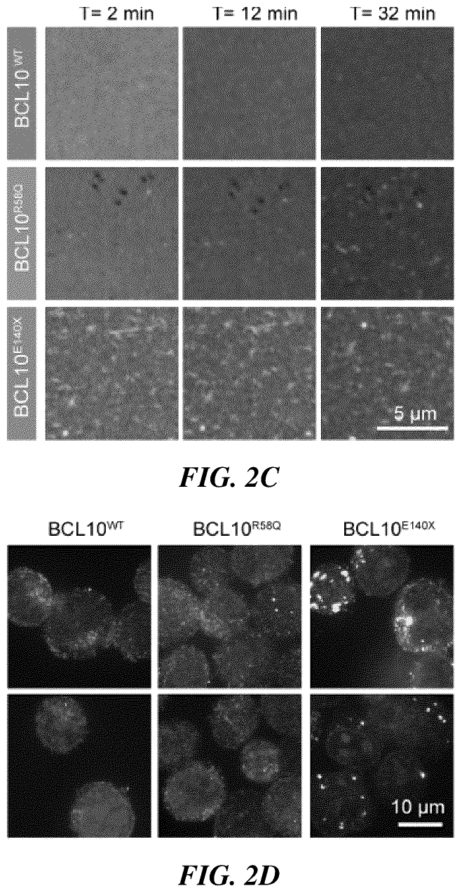

To determine the mechanism through which BCL10 mutants might confer a biochemical gain of function, purified full-length BCL10^{WT}, BCL10^{E140X} and BCL10^{R58Q} proteins were fused to a 3C protease-cleavable maltose binding protein (MBP) at the N-terminus were expressed to keep the proteins in a monomeric state (FIG. 2A). The purified proteins were labeled with Alexa488 through cysteine residues in the unstructured C-terminal region. Spontaneous filament formation was then assessed by confocal fluorescence microscopy as a function of BCL10 concentration 4 h after mixing with the 3C protease to remove the MBP tag.

Judging from the images obtained (some shown in FIG. 2B), BCL10^{WT} had a concentration of polymerization at ˜0.5 μM while BCL10^{R58Q} initiated filament formation at a somewhat lower concentration of ˜0.25 μM. Strikingly, BCL10^{E140X} started to form filaments even at the lowest concentration tested of 0.1 μM, indicating greatly enhanced ability to polymerize spontaneously (FIG. 2B). Of note, even MBP-fused BCL10^{E140X} in which the maltose binding protein (MBP) should sterically suppress filament formation, exhibited a significant polymerized fraction during protein purification. In contrast, we did not observe this phenomenon for BCL10^{WT} or BCL10^{R58Q}.

The kinetics of filament formation by these proteins was further evaluated at 1 μM concentrations, which are above the concentration of polymerization for WT and these mutant BCL10 proteins, using time lapse confocal fluorescence microscopy. As illustrated in FIG. 2C, the BCL10^{E140X} protein was already extensively polymerized at 2 min post-mixing with 3C protease, the earliest time point that could be imaged. While filament formation kinetics of BCL10^{WT} and BCL10^{R58Q} were both slower than that of BCL10^{E140X}, BCL10^{R58Q} showed more apparent filament formation at 32 min after addition of the 3C protease than BCL10^{WT} (FIG. 2C). These results indicate that BCL10^{E140X} and to a lesser extent BCL10^{R58Q} accelerated BCL10 polymerization threshold and kinetics.

Activated CARD11-BCL10-MALT1 (CBM) complexes manifest as puncta when visualized through confocal microscopy of living cells (Lenz et al., Science 319:1676-1679 (2008); Rossman et al., Mol Biol Cell 17:2166-2176 (2006); Traver et al., Methods Mol Biol 1584:101-127 (2017)). Therefore, ABC-DLBCL cells (HBL1) were imaged that had been engineered for constitutive expression of FLAG-tagged BCL10^{WT}, BCL10^{E140X} and BCL10^{R58Q}, respectively. These experiments revealed large, striking aggregates of BCL10^{140X}, in comparison with the much smaller puncta of BCL10^{E140X} or BCL10^{R58Q} (FIG. 2D). These results support the in vitro results on the recombinant proteins. The aggregates are reminiscent of the large puncta of an oncogenic, gain-of-function CARD11 mutant (Lenz et al., Science 319:1676-1679 (2008); Stinson et al., Cell Immunol 353:104129 (2020)), indicating that these aggregates represent active CBM complexes.

### Example 4: The R58Q Mutant Forms Filaments with a Glutamine Ladder, Enhanced Stability and Tendency to Bundle

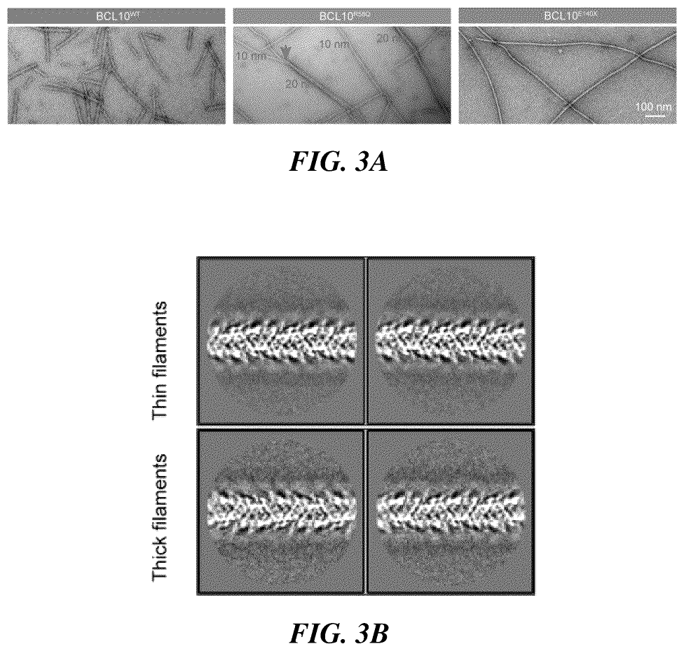

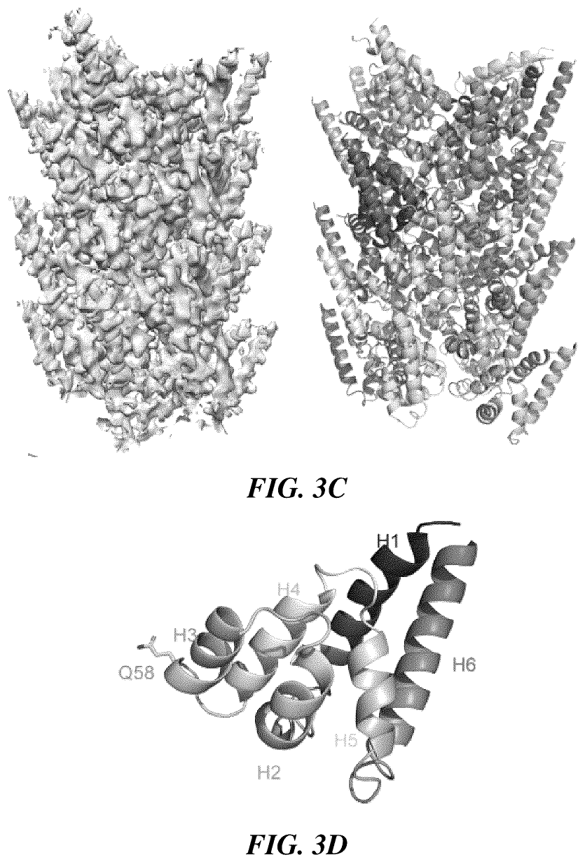

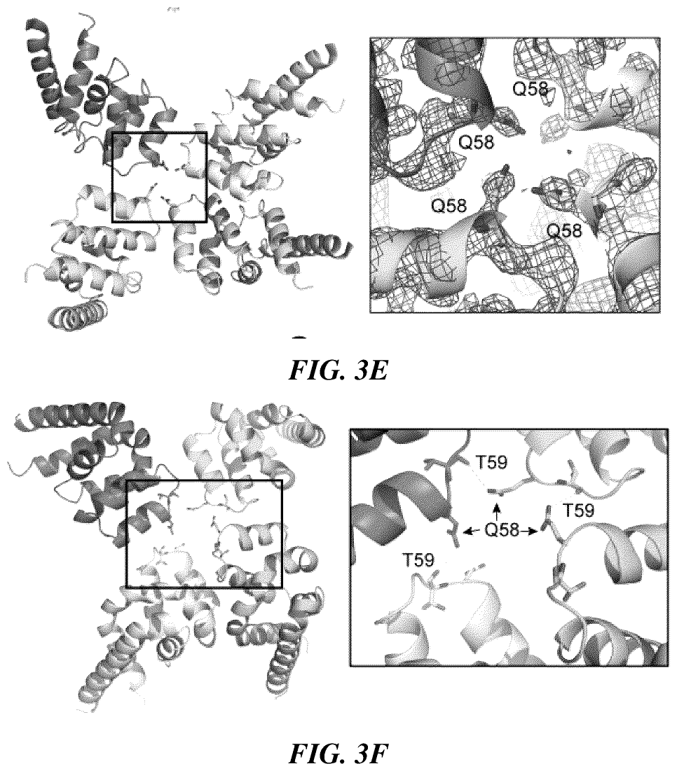

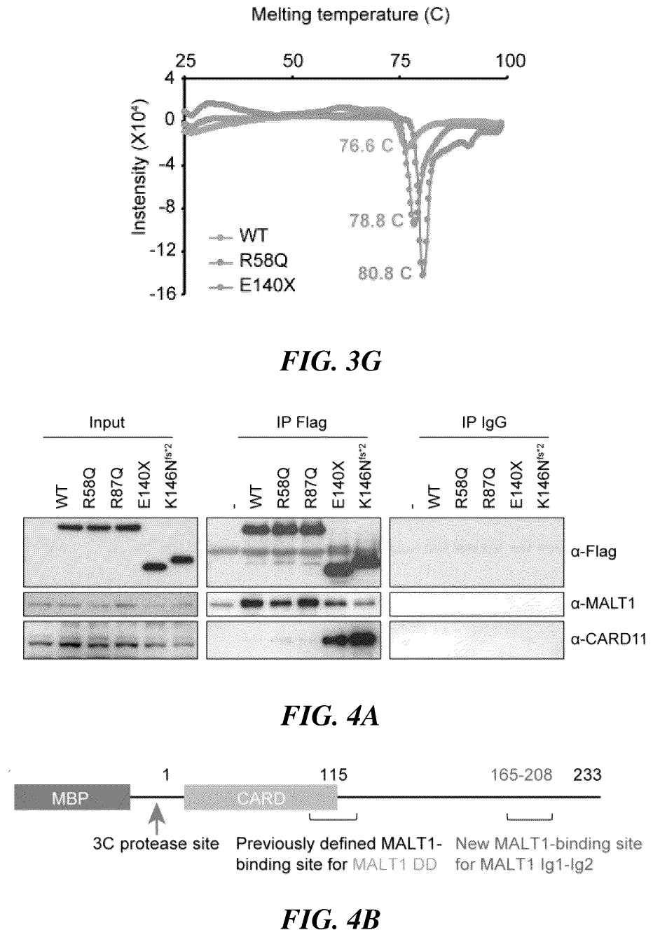

To better visualize these filaments, BCL10^{WT}, BCL10^{R58Q} and BCL10^{E140X} filaments were treated with 3C protease and purified by collecting the void fraction (i.e. polymerized BCL10) from a Superose 6 gel filtration column. The polymerized BCL10 filaments were then imaged using scanning electron microscopy (EM) (FIG. 3A). Surprisingly, whereas BCL10^{WT} and BCL10^{E140X} formed the classical 10 nM filaments of CARDs, BCL10^{R58Q} formed both 10 nm filaments and thicker 20 nM filaments (FIG. 3A). Closer inspection of the 20 nM filaments suggested that they are bundled 10 nM filaments because these thinner filaments were observed to merge into thicker filaments (FIG. 3A, arrow).

To determine if the BCL10R^{5R}Q filaments are structurally different from BCL10 or BCL10^{E140X} filaments and how the bundling occurs, cryo-EM data were collected using an Arctica microscope operating at 200 keV and a K2 electron counting direct detection camera. Thin and thick filaments were manually selected from the cryo-EM micrographs. As illustrated in FIG. 3B, such two dimensional (2D) classification revealed average filaments of similar thickness. These data indicated that the association between filaments within the bundle is not specific so that only one thin filament within each thick filament could be aligned and the other filament was averaged out. Thus, the thick filaments consist of randomly bundled thin filaments (FIG. 3B).

Using 3D reconstruction, the cryo-EM structure of the BCL10^{R58Q} filament was determined at 4.6 Å resolution as assessed by gold-standard Fourier shell correlation (FSC). The overall structure was similar to that of the BCL10^{WT} filament with only the CARD domain ordered (FIG. 3C). The Q58 residue resides in helix 3 (H3) of the six-helical bundle fold of the CARD domain (FIG. 3D). Within the BCL10^{R58Q} filament structure, Q58 localizes near the center and its side chain density is well defined (FIG. 3E), which contrasts with the poor density of the equivalent wild type R58 in the BCL10^{WT} filament structure. While the resolution of the structure was limited, Q58 residues appeared to reside at the center of the filament formed stacks of glutamine residues, with direct and potentially water-mediated interactions. In the center of the filament, the Q58 side chain (NE2 atom) of one protomer in the filament formed a potential hydrogen bond with the carbonyl oxygen of T59 of the next protomer in the helical filament (FIG. 3F).

To further demonstrate the potential role of the hydrogen bonding network in BCL10 filament assembly within the R58Q mutant, an R58E mutant was generated that does not have the NE2 atom (e.g. Q58 side chain) for hydrogen bond formation. The biochemical and biophysical properties of the R58E mutant were then characterized. However, there was no enhancement of filament formation for R58E and the concentration for R58E polymerization was about 1 μM, which was even higher than WT BCL10. These results confirm that the hydrogen bond formed by Q58 is important for its filament formation.

These Q58-mediated interactions prompted further experiments designed to determine whether there might be a difference in the stability of the BCL10^{R58Q} filament in comparison to BCL10^{WT} and BCL10^{E140X} filaments. To assess this possibility, thermal melt assays were performed on purified BCL10^{WT}, BCL10^{R58Q} and BCL10^{E140X} filaments. As shown in FIG. 3G, BCL10^{R58Q}, and to a lesser degree BCL10^{E140X}, yielded more stable filaments, with thermal melting temperatures of 80.8° C. and 78.8° C. respectively, in comparison with 76.6° C. for the BCL10w. Thus, while BCL10^{E140X} shows enhanced polymerization, BCL10^{R58Q} forms more stable filaments, which may explain its tendency to bundle.

### Example 5: Loss of Basal MALT1 Binding Promotes Spontaneous Polymerization of BCL10E14X

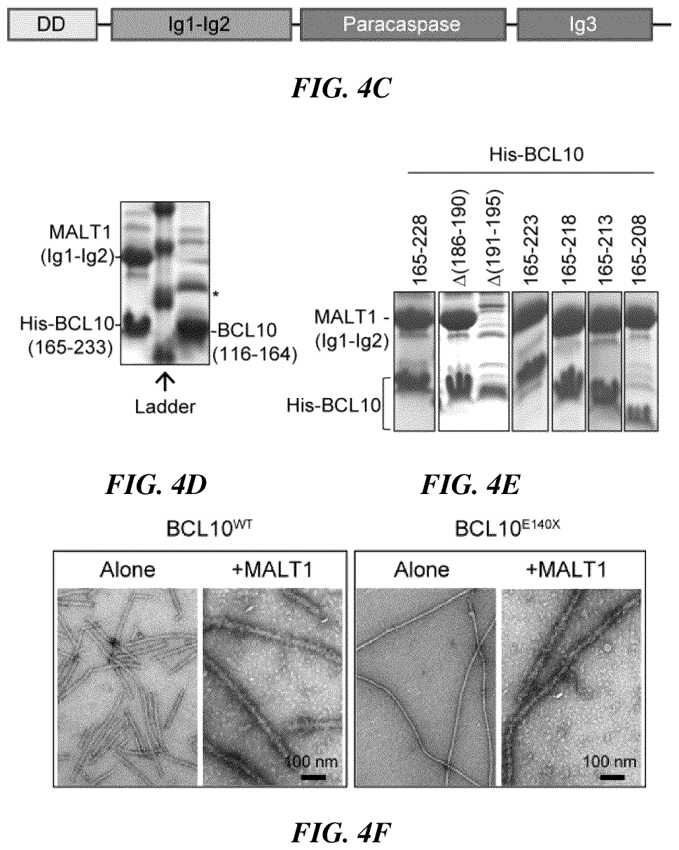

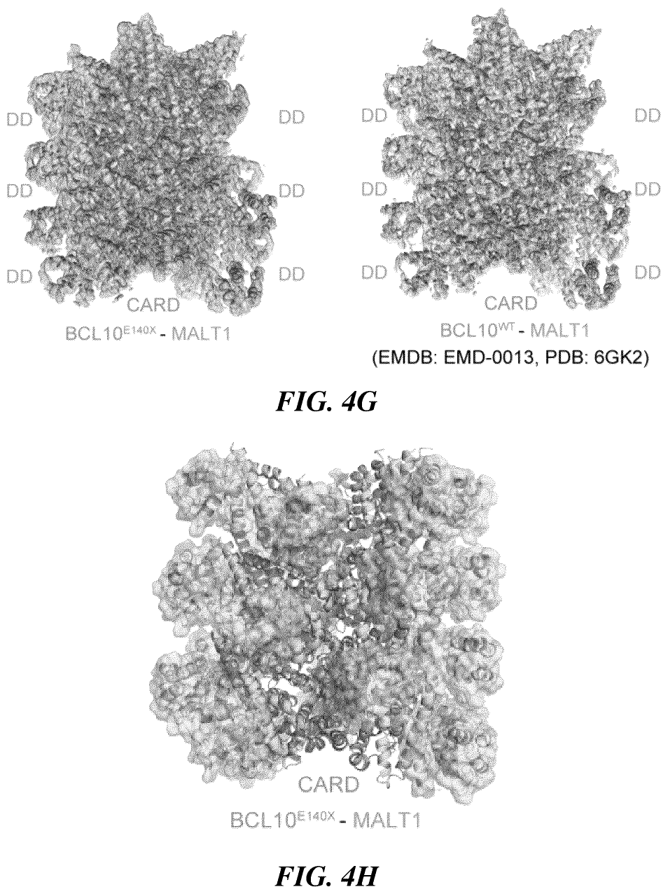

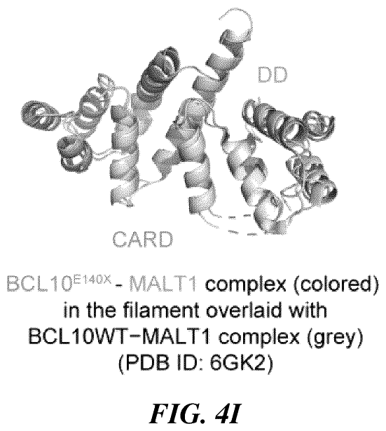

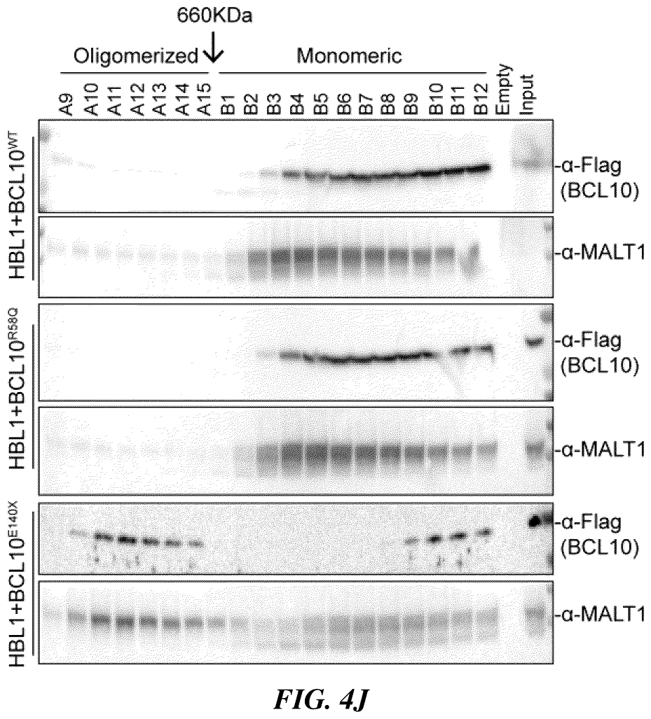

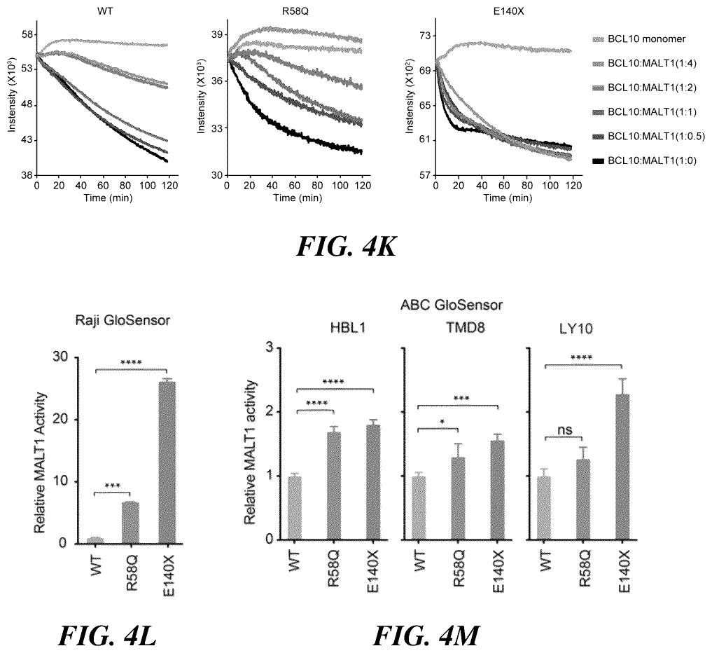

To investigate how the truncation mutants might affect CBM complex formation, Flag-tagged wild type and mutant forms of BCL10 were expressed in Raji cells, which lack constitutive B-cell receptor (BCR) signaling. Anti-Flag co-immunoprecipitations were performed on cell lysates. As illustrated in FIG. 4A, equivalent enrichment for MALT1 was observed in WT and BCL10^{R58Q} as well as in another CARD missense mutant BCL10R87Q. In contrast, there was less binding of MALT1 to BCL10^{E140X}, as well as to the similar BCL10^{K146Nfs*2} truncation mutant (FIG. 4A). While BCL10 CARD mutants interacted with CARD11 marginally better than BCL10^{WT}, the C-terminal mutants manifested much greater CARD11 interaction (FIG. 4A), which is likely due to the increased spontaneous polymerization of truncated BCL10, because BCL10 polymers have been shown to enhance interaction with CARD11 (Cheng et al. Cell Immunol 355:104158 (2020)).

The observed weaker recruitment of MALT1 by BCL10 truncation mutants (FIG. 4B) was surprising in view of previous studies that have mapped the BCL10 CARD as a MALT1 interacting domain by mutagenesis and cryo-EM structure of the BCL10-MALT1 filamentous complex (Schlauderer et al. Nat Commun 9:4041 (2018); Koseki et al. J Biol Chem 274:9955-9961 (1999)).

MALT1 has multiple domains (FIG. 4C) and in the reported cryo-EM structure, only the MALT1 death domain (DD) was ordered and interacted with the BCL10 CARD (residues 1-115), whereas the MALT1 immunoglobulin-like domains (Ig1-Ig2) and the paracaspase domain were not visible (Schlauderer et al. Nat Commun 9:4041 (2018)). A previous mapping study suggested that the Ig1-Ig2 domains of MALT1 could also interact with BCL10 (Lucas et al. J Biol Chem 276:19012-19019 (2001)).

The inventors designed experiments to determine whether there were additional MALT1-binding sites on BCL10 at its largely unstructured C-terminus. The C-terminal region was divided into two halves and MALT1 pull-down studies were performed. As shown in FIG. 4D, the second half (residues 165-233) of the BCL10 C-terminus, but not the first half (residues 116-164), pulled down the Ig1-Ig2 construct of MALT1 when co-expressed in E. coli shown by Coomassie blue-stained SDS-PAGE gel. Further truncations of the BCL10 165-233 fragment showed that a construct containing residues 165-208 was sufficient to pull down MALT1 Ig1-Ig2 (FIG. 4E), confirming this is a second MALT1-binding site (FIG. 4B). Of note, unlike the interaction between BCL10 CARD and MALT1 death domain (DD) in the filamentous form, the interaction between BCL10 C-terminal region and MALT1 Ig1-Ig2 is monomeric as assessed by gel filtration chromatography of the complex.

This new MALT1 binding domain is deleted from BCL10^{E140X}. The inventors next explored whether there was any impairment in MALT1 recruitment to BCL10 filaments, by generating BCL10^{WT} and BCL10^{E140X} filaments in vitro and incubating them with purified, full length MALT1 followed by negative staining electron microscopy (EM). These experiments showed equivalent patterns of MALT1 decorating the surface of WT and BCL10^{E140X} filaments, suggesting that MALT1 recruitment was intact in each case (FIG. 4F). However, this experiment may not provide sufficient resolution to show whether MALT1 distribution on the different BCL10 polymers was altered. Therefore, cryo-EM data were then collected on a Titan Krios microscope operating ats 300 keV and equipped with a Falcon II direct electron detector. The cryo-EM structure of BCL10^{E140X} filaments in complex with MALT1 was thus determined at 4.3 Å resolution.

The cryo-EM structure of the BCL10^{E140X} filament with MALT1 was highly similar to that of the BCL10^{WT} filament with MALT1 at 4.9 Å resolution, in which the death domains of MALT1 bind the CARD of BCL10 and decorate the outside of the core CARD filament (FIG. 4G-4I). The conserved structure confirmed that the truncation did not affect the association of BCL10 filaments with MALT1, but that BCL10^{E}l4× is nonetheless defective in interacting with monomeric MALT1.

To further investigate these associations, gel filtration analysis was performed from lysates of ABC-DLBCL cells expressing FLAG-tagged BCL10^{WT}, BCL10^{R58Q} or BCL10^{E140X}. Cell fractionation of these lysates revealed a relatively small proportion of BCL10^{WT} or BCL10^{R58Q} in high molecular weight fractions corresponding to filaments, along with a small fraction of MALT1, whereas most BCL10 and MALT1 proteins were in low molecular weight fractions (FIG. 4J). In marked contrast, BCL10^{E140X} was present at higher abundance in high molecular weight fractions, with corresponding enrichment of MALT1 (FIG. 4J). Reciprocally, there was reduced abundance of BCL10^{E140X} and a notable reduction of MALT1 in the lower molecular weight complexes. Similar findings were observed by performing sucrose gradient experiments.

The association between enhanced polymerization and lack of MALT1 monomeric interaction of BCL10^{E140X} prompted the inventors to hypothesize that MALT1 binding to the C-terminal region of BCL10 might inhibit BCL10 polymerization. To investigate whether this was the case Alexa488-labeled, MBP-fused BCL10^{WT}, BCL10^{R58Q} or BCL10^{E140X} were incubated with increasing concentrations of purified MALT1. These reaction mixtures were then treated with C3 protease to remove the MBP moiety, and the BCL10 polymerization kinetics were monitored using fluorescence quenching.

As shown in FIG. 4K, increasing doses of MALT1 suppressed BCL10^{WT} and BCL10^{R58Q} filament formation in a dose dependent manner. In contrast, there was little suppression of BCL10^{E140X} polymerization by MALT1 at any dose (FIG. 4K). Taken together these data indicate that BCL10^{E140X}, and other similar C-terminal truncation mutations, favor BCL10 polymerization in cells not only by reducing its intrinsic concentration threshold (FIG. 2B-2C), but also by abrogating a novel MALT1 inhibitory effect mediated through interaction with the novel BCL10-MALT1 binding site in the C-terminal region of wild type BCL10v (FIG. 4K). By the same token, loss of C-terminal tail binding by BCL10^{E140X}, and other similar C-terminal mutations, would increase the pool of MALT1 that is available to bind to BCL10 polymers. The end result would be potent enhancement of BCL10 filament formation and MALT1 activity.

### Example 6: Differential Activation of MALT1 by BCL10^{E140X} Vs BCL10^{R58Q}

Because MALT1 dimerization on BCL10 filaments activates its proteolytic function, the inventors designed experiments to determine whether skewing of MALT1 cellular pools towards the BCL10 polymer bound state in the BCL10^{E140X} setting might lead to higher cellular levels of MALT1 activity.

The effect of BCL10 mutants on MALT1 activity was examined within cells in the absence of basal BCR signaling. To do this, MALT1 enzymatic reporter assays were performed, using a GloSensor protein construct engineered with a specific MALT1 cleavage site (Fontan et al., J Clin Invest 128:4397-4412 (2018)) stably transduced into Raji cells expressing BCL10^{WT}, BCL10^{E140X} or BCL10^{R58Q}).

As shown in FIG. 4L, significantly greater MALT1 enzymatic activation was observed in BCL10^{E140X} cells compared to MALT1 enzymatic activation in BCL10^{WT} and BCL10^{R58Q} cells. Expression of the BCL10^{R58Q} mutant also led to greater MALT1 activity when compared to BCL10^{WT}, albeit to lesser extent than the BCL10^{E140X} mutant. These results are consistent with the slightly enhanced polymerization kinetics of BCL10⁵⁸.

Similar MALT1 protease reporter assays were performed in ABC-DLBCL cell lines expressing BCL10^{WT}, BCL10^{E140X} or BCL10^{R58Q}, where there is constitutive activation of signaling to the CBM complex. Again BCL10^{E140X} generally yielded the strongest MALT1 activation (FIG. 4M), while BCL10^{R58Q} generally yielded greater MALT1 activity than WT BCL10.

Collectively, these data indicate that both BCL10^{R58Q} and especially BCL10^{E140X}, through distinct mechanisms, lead to aberrantly increased MALT1 activity.

### Example 7: BCL10^{E140X} Confers Reduced Dependency on CARD11 for CBM Activation

Normally, active CARD11 is required to nucleate the formation of BCL10 filaments (Qiao et al., Mol Cell 51:766-779 (2013); David et al., Proc Natl Acad Sci USA 115:1499-1504 (2018)). However, the inventors hypothesized that the requirement for CARD11 could be diminished in ABC-DLBCL cells expressing BCL10^{E140X}, given its greater tendency to polymerize and loss of MALT1 inhibitory interactions.

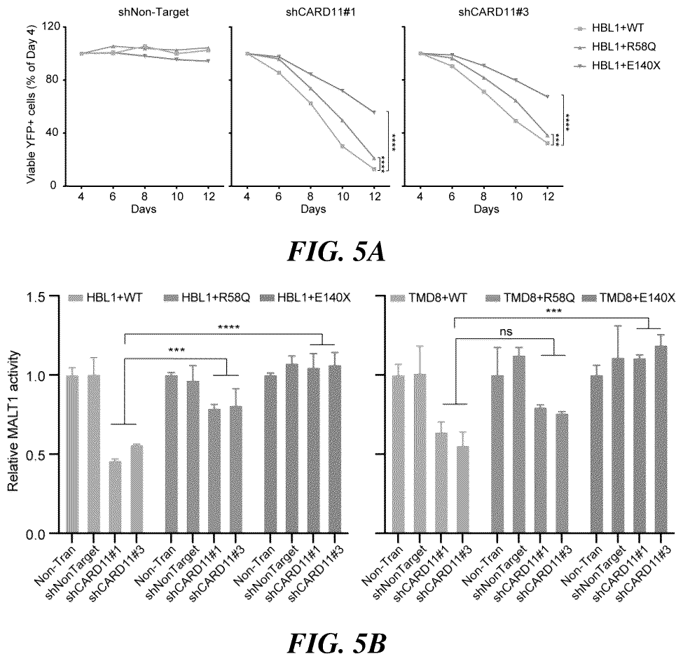

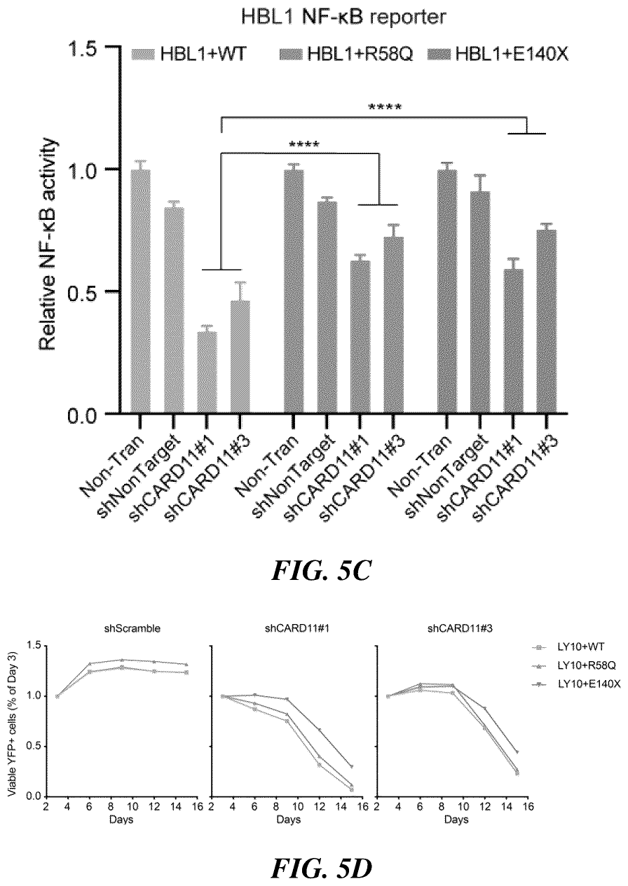

To test this hypothesis, CARD11 shRNA knockdown experiments were performed in isogenic ABC-DLBCL cells expressing BCL10^{WT}, BCL10^{R58Q} and BCL10^{E140X}. CARD11 depletion can cause proliferation arrest of ABC-DLBCL cells (Lenz et al., Science 319:1676-1679 (2008)) As shown in FIG. 5A, CARD11 knockdown induced significant growth suppression when wild type BCL10 was expressed (FIG. 5A, 5D). However, this effect was significantly blunted in when BCL10^{E140X}, and to a lesser extent when BCL10^{R58Q}, was expressed.

To determine how the effects of these mutant BCL10 proteins relate to CBM complex function, the impact of CARD11 knockdown on MALT1 activity was evaluated using GloSensor reporter assays. MALT1 activity was highly impaired after CARD11 knockdown in the presence of wild type BCL10, but this effect was completely rescued in BCL10^{E140X} cells and partially rescued by BCL10^{R58Q}(FIG. 5B). CARD11 knockdown also reduced NF-κB reporter activation in BCL10^{WT} ABC-DLBCL cells, an effect that was blunted in CARD11 depleted in ABC-DLBCL cells expressing BCL10^{R85Q} and BCL10^{E140X} (FIG. 5C).

As illustrated in the foregoing Example, BCL10^{E140X} activates MALT1, which may be linked to its reduced requirement for CARD11 to induce filament formation. These results are consistent with the data described herein showing markedly greater BCL10^{E140X} activity in unstimulated B-cells. The BCL10^{R58Q} mutant, which does not polymerize as readily as BCL10^{E140X} and is still inhibited by MALT1 monomers, retained a greater degree of CARD11 dependency.

### Example 8: BCL10^{R58Q} and BCL10^{E140X} Confer Distinct Levels of Resistance to Ibrutinib

Bruton's tyrosine kinase (BTK) inhibitors have emerged as a precision therapy modality for ABC-DLBCLs (Wilson et al., Nat Med 21:922-926 (2015); Aalipour & Advani, Ther Adv Hematol 5:121-133 (2014)). However, lymphoma cells with inherent or acquired mutations in activating proteins downstream of BTK (e.g. those CARD11 mutations that induce potent MALT1 activation) can be resistant to such treatments (Wilson et al., Nat Med 21:922-926 (2015); Caeser et al., JCO Precis Oncol 5:145-152 (2021)).

Given the distinct functional profiles, CARD11 dependencies, and MALT1 activation effects of the BCL10 CARD and truncation mutants, the inventors designed experiments to evaluate whether, and to what extent, these mutant BCL10 proteins might confer BTK inhibitor resistance. Isogenic ABC-DLBCL cells expressing BCL10WT, BCL10^{R58Q} and BCL10^{E140X} proteins were treated with escalating doses of three chemically distinct covalent BTK inhibitors: ibrutinib, acalabrutinib, or zanubrutinib. The cellular proliferation rates of the treated cells were measured using an ATP fluorescence assay after 96 hours of drug exposure.

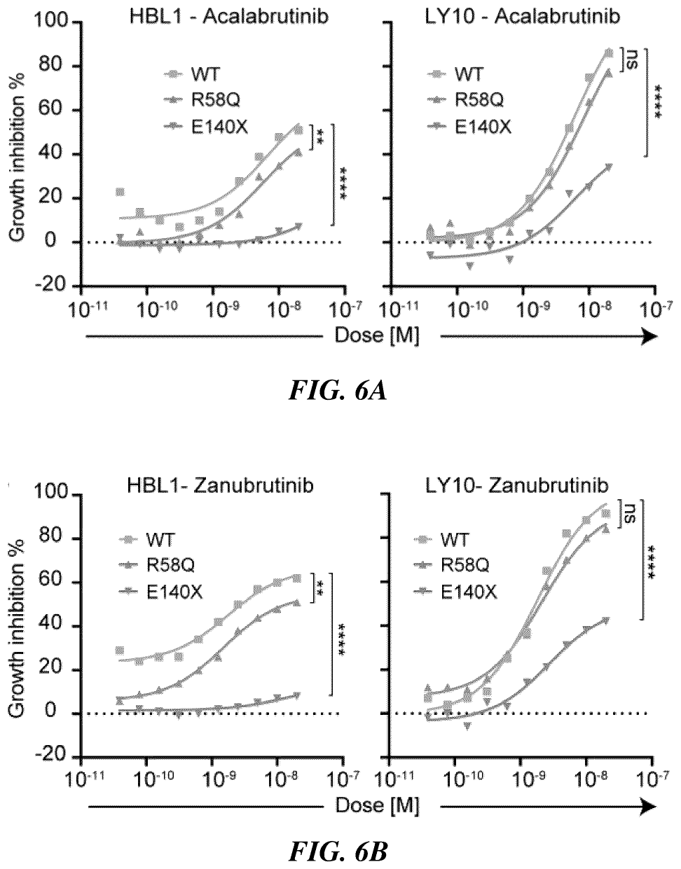

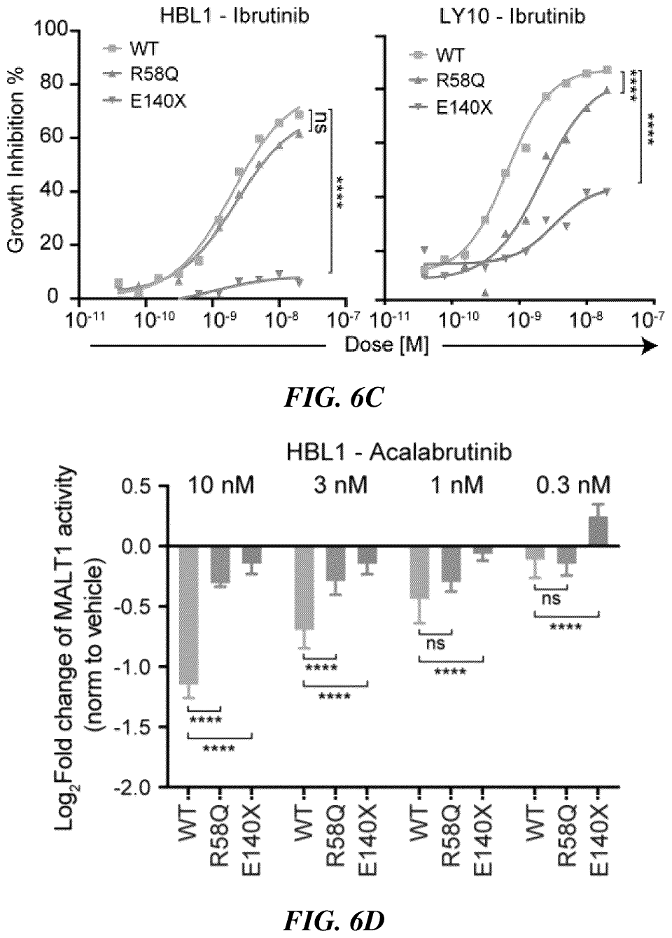

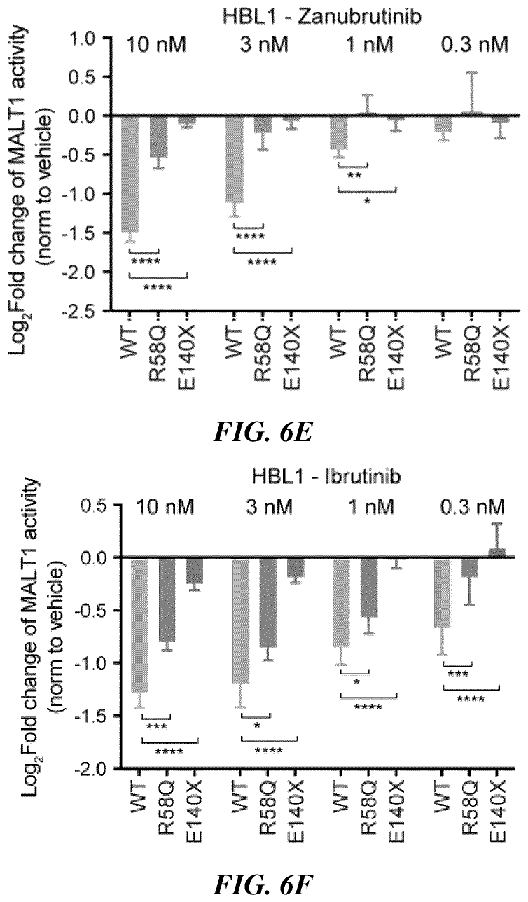

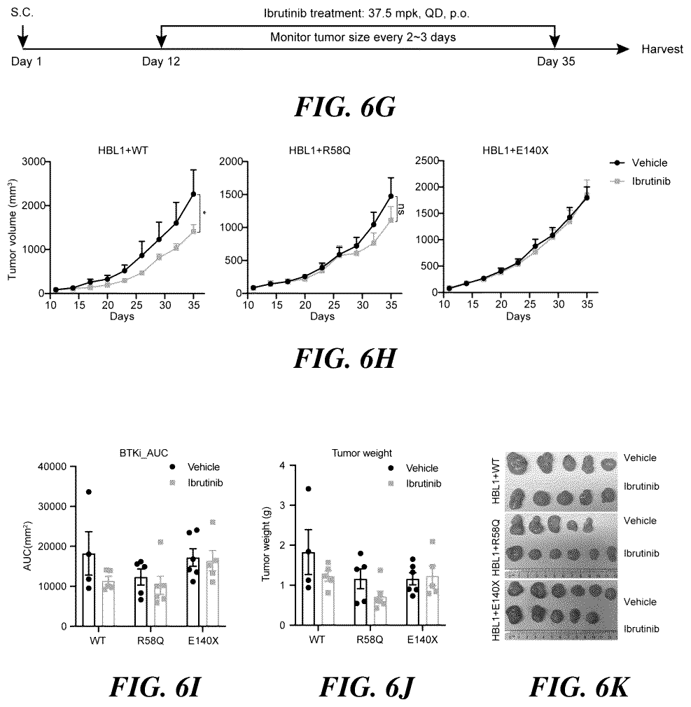

As shown in FIG. 6A-6C, BCL10^{R58Q} conferred at least a modest and often significant reduction in response to these drugs. In contrast, BCL10^{E140X} conferred far more dramatic resistance in almost all cases (FIG. 6A-6C). The IC50 values of the BTK inhibitors in human diffuse large B-cell lymphoma (HBL1) and in OCI-Ly10 (ABC-DLBCL tumor) cells that expressed wild type or mutant BCL10 are shown below.

| | Acalabrutinib | Zanubrutinib | Ibrutinib |
|---|---|---|---|
| | (nM) | (nM) | (nM) |
| HBL1^{WT} | 4.75 ± 2.11 | 1.46 ± 0.32 | 4 |
| HBL1^{R58Q} | 5.54 ± 0.32 | 1.58 ± 0.06 | 6.1 |
| HBL1^{E140X} | 28.2 ± 21.2 | 9.31 ± 6.22 | 11 |
| OCI-LY10^{WT} | 6.38 ± 0.35 | 1.74 ± 0.08 | 0.66 |
| OCI-LY10^{R58Q} | 7.69 ± 0.59 | 1.81 ± 0.33 | 2.2 |
| OCI-LY10^{E140X} | 5.39 ± 0.77 | 2.86 ± 0.13 | 3.3 |

As illustrated in FIG. 6D-6F, all three BTK inhibitors suppressed MALT1 activity in BCL10^{WT} ABC-DLBCL cells in a potent and dose dependent manner. However, isogenic BCL10^{R58Q} and BCL10^{E140X} ABC-DLBCL cells manifested significantly less impact on MALT1, especially in the case of BCL10^{E140X} (FIG. 6D-6F).

When the further downstream impact of the BTK inhibitors was measured on NF-κB reporter activity, significantly blunted NF-κB reporter activity was observed in cells expressing BCL10^{R58Q} and BCL10^{E140X}.

Ibrutinib (37mpk, oral gavage, Q.D.) was administered to mice bearing BCL10^{WT}, BCL10^{R58Q} and BCL^{E140X} expressing ABC-DLBCL xenografts and the tumor volumes in the different mice were measured. As shown in FIG. 6G, ibrutinib suppressed growth of BCL10^{WT} ABC-DLBCL tumors, but had no significant anti-tumor effect against the two mutant forms. FIG. 6H-6K further illustrate the effects (or lack thereof) of ibrutinib on mice bearing BCL10^{WT}, BCL10^{R58Q} and BCL10^{E140X} expressing ABC-DLBCL xenografts, with the BCL10^{E140X}-expressing xenografts being most resistant to ibrutinib. Collectively, BCL10^{R58Q} and BCL10^{E140X} confer distinct levels of resistance to BTK inhibition, consistent with their different mechanisms of action and impact on MALT1 activation.

### Example 9: BCL10 Truncating Mutant Lymphomas are Hypersensitive to MALT1 Protease Inhibitor

The fact that BCL10 mutants drive potent MALT1 activation even in the absence of CARD11 and that these BCL10 mutants confer reduced responses to ibrutinib led the inventors to hypothesize that cells expressing BCL10 mutants might be especially dependent on MALT1 and hence highly responsive to MALT1 inhibitors.

To evaluate this hypothesis, the impact of three chemically and mechanistically distinct MALT1 inhibitors was tested against the inventors' set of isogenic ABC-DLBCL cells. The MALT1 inhibitors tested included:

C3, a potent and specific compound that covalently inactivates the MALT1 catalytic pocket (Fontan et al., J Clin Invest 128:4397-4412 (2018));

MLT-748, a reversible allosteric compound that binds MALT1 Trp580 side chain thus to lock protease inactive (Quancard et al., Nat Chem Biol 15:304-313 (2019)); and

JNJ-67690246, an allosteric MALT1 inhibitor, having the structure shown below.

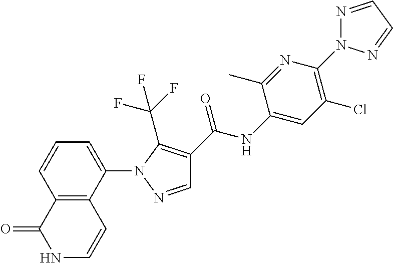 [SMILES: Cc1nc(-n2nccn2)c(Cl)cc1NC(=O)c1cnn(-c2cccc3c(=O)[nH]ccc23)c1C(F)(F)F]

JNJ-67690246 potently inhibits MALT1 enzymatic activity (IC50=15 nM) in biochemical assays and cytokine secretion of IL6/10 (IC50=60 nM) in OCI-Ly3 cellular assays, as shown by the IC50 values in the table below.

| | JNJ-67690246 | | IC₅₀ (nM) |
|---|---|---|---|
| MALT1 enzyme | | | 15 |
| | OCI-Ly3 | IL6 | 60 |
| | | IL10 | 60 |
| MALT1 enzyme | | | 15 |
| | OCI-Ly3 | IL6 | 60 |
| | | IL10 | 60 |

Isogenic BCL10^{WT}, BCL10^{R58Q} and BCL10^{E140X} ABC-DLBCLs were exposed to increasing concentrations of each of these compounds for 96 hours.

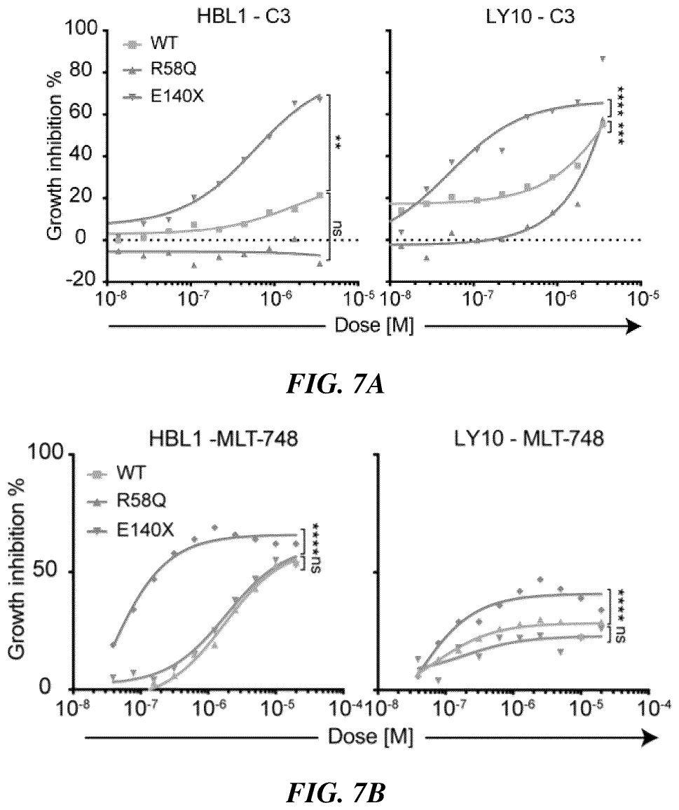

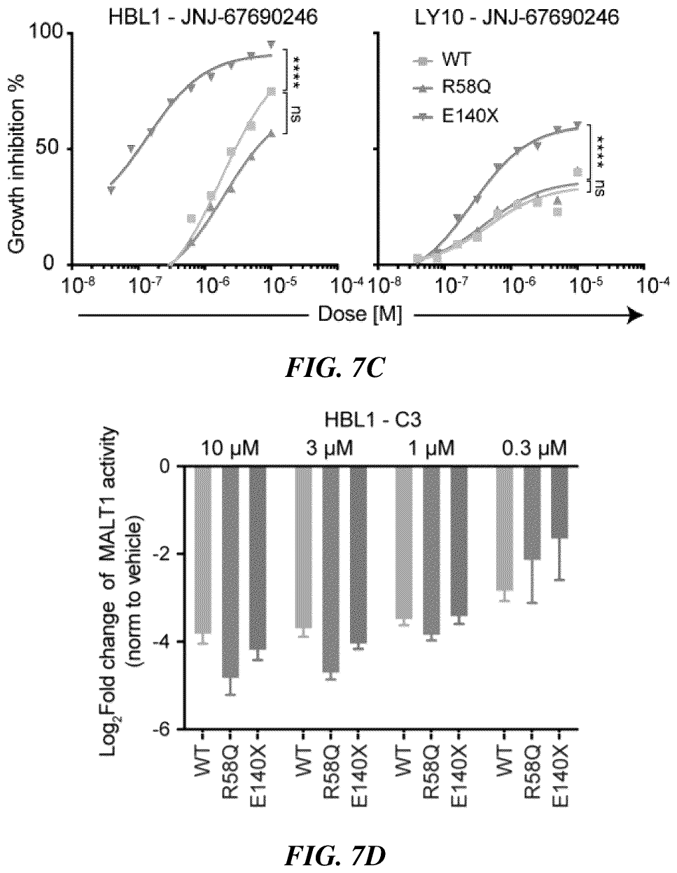

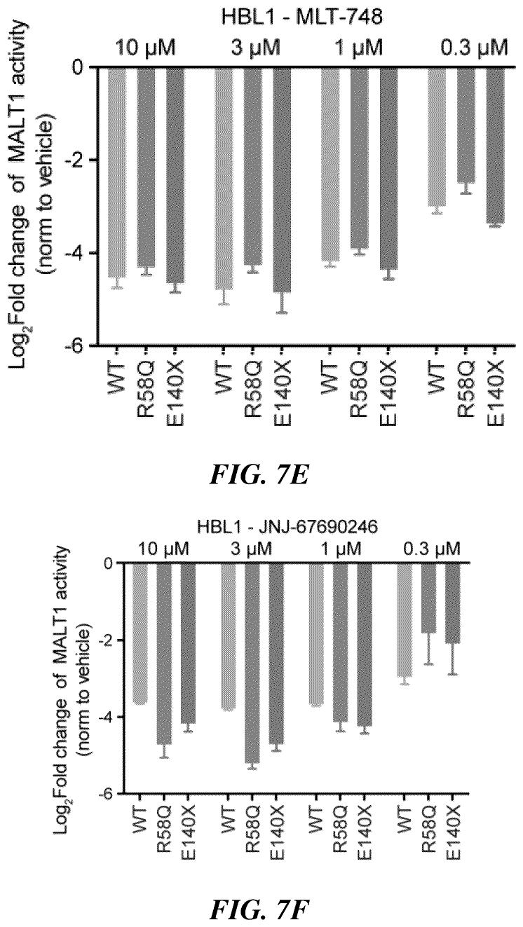

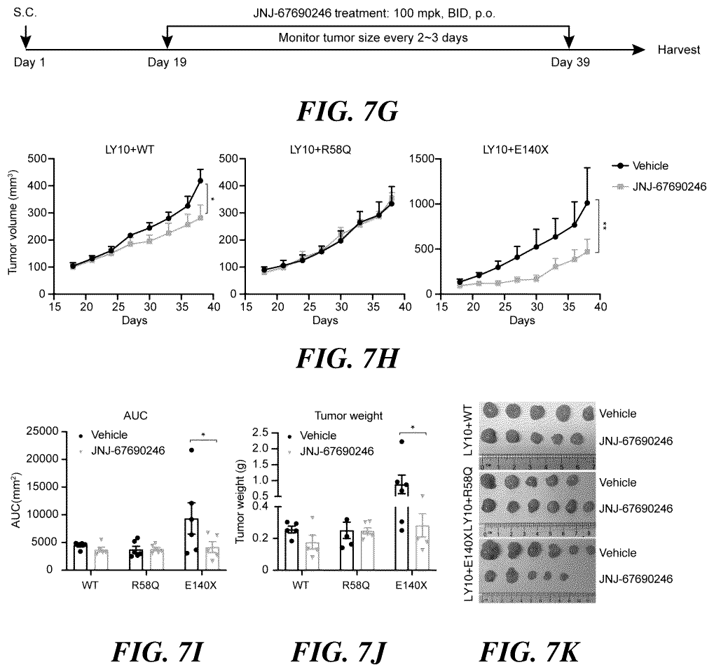

As shown in FIG. 7A-7C, there were striking differences in the response profiles of BCL10^{E140X} vs BCL10^{WT} and BCL10^{R58Q}. The table below summarize the IC50 values for the MALT1 inhibitors in the different cell types.

| | MLT-748 (uM) | JNJ-67690246 (uM) | C3 (uM) |
|---|---|---|---|
| HBL1^{WT} | 1.94 ± 0.2 | 1.58 ± 0.5 | 1.7 |
| HBL1^{R58Q} | 3.32 ± 1.46 | 5.77 ± 3.02 | NA |
| HBL1^{E140X} | 0.54 ± 0.5 | 0.15 ± 0.02 | 0.59 |
| OCI-LY10^{WT} | 0.07 ± 0.006 | 0.32 ± 0.07 | 7.3 |
| OCI-LY10^{R58Q} | 0.24 ± 0.05 | 0.78 ± 0.2 | NA |
| OCI-LY10^{E140X} | 0.09 ± 0.06 | 0.27 ± 0.02 | 0.05 |

Both WT cells and BCL10^{R58Q} cells were generally sensitive to the allosteric inhibitors (MLT-748 and JNJ-67690246), but BCL10^{R58Q} cells were less sensitive than wild type to C3 (FIG. 7A-7C). In marked contrast, all three inhibitors were markedly more effective in BCL10^{E140X} cells, leading to significantly greater MALT1 inhibition at lower doses. This differential effect was not due to variation in the degree of MALT1 inhibition, because MALT1 activity was equivalently suppressed by all three drugs in BCL10^{WT}, BCL10^{R58Q} and BCL10^{E140X} ABC-DLBCL cells (FIG. 7D-7F).

Analysis of NF-κB reporter activity showed significantly greater impairment in MALT1 inhibitor treated BCL10^{E140X} cells as compared to either BCL10^{WT} or BCL10^{R58Q}. Cells expressing BCL10^{R58Q} exhibited even less impairment of the NF-κB activity than in wild type cells. These results indicate that some other pathway may be maintaining NF-κB and hence conferring less dependency on MALT1 than in BCL10^{E140X} cells.

Mice having BCL10^{WT}, BCL10^{R58Q} and BCL10^{E140X} ABC-DLBCL xenografts were treated in vivo with JNJ-67690246, using the OCI-Ly10 cell line, which is generally less sensitive to MALT1 inhibition (FIG. 7A-7C, 7G), for greater stringency. As shown in FIG. 7H-7K, significant reduction in growth of BCL10^{E140X} lymphomas was observed but not BCL10^{WT} or BCL10 lymphomas. These results confirm the increased dependency on MALT1 activity for these MALT1 inhibitors, and illustrating that MALT1 inhibitors can be most useful for patients bearing BCL10^{R58Q} and BCL10^{E140X} mutations.

### Results

BCL10, one of the most frequently mutated genes in DLBCL, is a bona fide genetic driver of lymphomagenesis. Importantly, structure-function studies described herein reveal that specific mutations occur in at least two biochemically distinct classes within DLBCL patients: missense mutations of the CARD domain and truncation mutations of the C-terminal tail. These classes of mutations seem to affect distinct aspects of BCL10 functionality and lead to biologically distinct outcomes as indicated by their differential downstream effects on MALT1 and NF-κB signaling as well as vulnerability to targeted therapies. Many of the BCL10 truncating mutations cluster between amino acid positions 135 to 174, and representatives of these mutations manifested the most powerful activation of NF-κB activity. BCL10 truncation mutants such as BCL10^{E140X} manifested a striking increase in its ability to polymerize into its filamentous form, accompanied by potent activation of MALT1 protease activity. This tendency to polymerize, indicated for example by its lower concentration threshold may help to explain the reduced CARD11 dependency of lymphoma cells expressing BCL10^{E140X}. Although CARD11 serves to nucleate BCL10 polymerization, CARD11 association with BCL10 filaments can be further stabilized by nascent helical BCL10 polymers, which is consistent with results showing increased association of BCL10 and CARD11 in lymphoma cells expressing BCL10^{E140X} in view of this mutant's greater tendency to polymerize, while at the same time being consistent with its reduced requirement for CARD11 to induce filament formation and reduced biological dependency on CARD11 in BCL10^{E140X} expressing DLBCL cells.

Binding of the MALT1 death domain to BCL10 was unperturbed in filaments composed of BCL10^{E140X}. This MAL T1-BCL10 molecular association is mediated through the BCL10 CARD domain and proximal regions that are not affected by C-terminal truncation mutations. However, co-immunoprecipitation experiments paradoxically indicated reduced interaction between BCL10 and MALT1. This prompted the inventors to examine other modes of BCL10-MALT1 association, leading to the identification of a novel direct interaction site between MALT1 Ig1-Ig2 region and BCL10 amino acid positions 165 to 208, a region that is lost in a majority of truncation mutants except for a cluster deleting the extreme C-terminal Ser/Thr rich tail. Importantly, the results described herein show that MALT1 impairs BCL10 polymerization through this interaction surface, thus constituting a novel CBM negative regulatory mechanism preventing spurious polymerization of BCL10. This explains the dramatically increased filament formation observed for BCL10^{E140X} in vitro, and greatly enhanced ability of the BCL10^{E140X} mutant to recruit MALT1 into the polymerized CBM complex, given that loss of monomeric BCL10 and MALT1 binding would increase the pool of MALT1 to associate with filaments. The events that occur due to the presence of BCL10^{E140X} thus appear to constitute a positive feedback loop that ultimately causes potent MALT1 protease activation and biological dependency on MALT1 catalytic function.

The more distal set of C-terminal truncating mutations such as Q208X, L209X and L225X retain the MALT1 Ig1-Ig2 interacting region and hence would not be expected to escape from this MALT1 inhibitory binding. Accordingly, when expressed in cells, they did not induce greater NF-κB activity than wild type BCL10, suggesting that there may be additional ways in which BCL10 function could be perturbed, perhaps due to specific loss of certain as of yet undiscovered post-translational modifications. Interestingly, a BCL10-L225X mutant produces a truncated form of BCL10 similar to the MALT1 protease dependent cleavage form of BCL10 R228X observed in activated T-cells as well as in ABC-DLBCL cells with chronic active BCR signaling and showed similar functional effects to wild type BCL10 overexpression in NF-κB reporter assays, perhaps due to retaining both intact MALT1 biding sites.

In contrast, missense mutations of the BCL10 CARD domain seem to have distinct functional effects. Many of the BCL10 residues affected by mutations (e.g. R58, K63) are localized within the core of BCL10 helical structures where they make important intrastrand (Type III) and interstrand (Type I) interactions that needed for filament formation. An R53Q mutation, affecting type III interactions, might be predicted to disrupt intrastrand interactions and caused a severe defect in BCL10 CARD domain polymerization. This was however proven to be incorrect, and the sole CARD domain hotspot mutant residue Q58 engages in a novel form of type III interaction resulting in apparent formation of a hydrogen bonded glutamine network. The consequence is a shift in BCL10 polymerization kinetics, favoring the polymerized state but without dramatically altering binding to CARD11 or MALT1 and yielding a more modest gain of function phenotype. Missense mutations such as K63Q might functionally resemble R58Q since they also locate at the central core of the BCL10 CARD filament, and have positively charged residues switched to glutamine to potentially enhance, rather than disrupt, interactions.

These biochemical features of the BCL10^{R58Q} mutant result in a hypomorphic phenotype compared to truncation mutant where they induced less potent MALT1 and NF-κB activation than BCL10^{E140X}. However, the results shown herein also indicate that BCL10^{R58Q} may engage in additional gain of function effects. Cells expressing BCL10^{R58Q} seemed relatively resistant to loss of NF-κB activity upon exposure to MALT1 inhibitors as compared to that on BCL10-WT or BCL10^{E140X}, whereas in contrast, reduction in NF-κB activity in response to the upstream BTK inhibitors was similar between BCL10^{R58Q} and BCL10^{E140X}. These data prompt us to hypothesize that other functions may derive from the stability or bundling of R58Q filament. For example, more stable BCL10 filaments might enhance MALT1 recruitment and activation of TRAF6, which could partially support NF-κB independently from the MALT1 proteolytic function, or activate NF-κB through other alternative means such as linear ubiquitylation (LUBAC) associated mechanisms. Engagement of these or other MALT1 paracaspase independent biochemical effects would be consistent with BCL10^{R58Q} DLBCL cells retaining greater dependency on CARD11 and hence upstream signaling and responsiveness to BTK inhibitors.

Overall, the data provided herein illustrate the complexities involved in developing precision therapies for DLBCLs and other tumors. The identification of chronic active BCR signaling as a characteristic of ABC-DLBCLs has led to intense efforts to integrate BTK inhibitors (BTKi) into multi-modality regimens. However to date, it is clear that many patients still do not benefit from the addition of such compounds. The results provided herein point to biochemical mechanisms that might help to explain this, as exemplified most clearly by the BTKi resistance conferred by BCL10 truncation mutants, indicating that such patients should not be treated with BTKi containing regimens. Instead, patients with BCL10 truncation mutations would likely best be served by incorporating MALT1 inhibitors. Several MALT1 inhibitors are already in clinical trials and can be used for this purpose. Similar considerations may apply to other (but not all) mutations downstream of BTK, as exemplified by the case of CARD11 coiled-coil domain mutants, which induce resistance to BTK inhibitors but not to MALT1 inhibitors. However, the fact that BCL10 CARD domain mutations may still retain BTK inhibitor responsiveness further underlines the need for rigorous study of signaling pathway mutations such as those described herein.

Example 10 ABC-DLBCLs have unfavorable outcomes and chronic activation of CBM signal amplification complexes that form due to polymerization of BCL10 subunits, which is affected by recurrent somatic mutations in ABC-DLBCLs. Herein, it is shown that BCL10 mutants fall into at least two functionally distinct classes: missense mutations of the BCL10 CARD domain and truncation of its C-terminal tail. Truncating mutation abrogated a novel motif through which MALT1 inhibits BCL10 polymerization, trapping MALT1 in its activated filament-bound state. CARD missense mutation enhanced BCL10 filament formation; forming glutamine network structures that stabilize BCL10 filaments. Mutant forms of BCL10 were less dependent on upstream CARD activation and thus manifested resistance to BTK inhibitors, whereas BCL10 truncating but not CARD mutants were hypersensitive to MALT1 inhibitors. Therefore, BCL10 mutations are potential biomarkers for BTK inhibitor resistance in ABC-DLBCL and further precision can be achieved by selecting therapy based on specific biochemical effects of distinct mutation classes.

ABC-DLBCLs feature frequent mutations of signaling mediators that converge on the CARD11-BCL10-MALT1 (CBM) complex. Herein structure-function approaches were used to reveal that BCL10 mutations fall into at least two distinct biochemical classes. Importantly both classes confer resistance to BTK inhibitors, whereas BCL10 truncating mutants confer hyper-responsiveness to MALT1 inhibitors, providing a roadmap for precision therapies for ABC-DLBCLs.

### Introduction

The CARD11/BCL10/MALT1 complex plays a role integrating signaling pathways involved in immunity and inflammation in a broad repertoire of cell types. In B-cells and T-cells, the CBM complex is activated downstream of B-cell receptor (BCR) or T-cell receptor (TCR) signaling and serves to amplify such signals leading to powerful phenotype responses conferred by downstream mediators. Accordingly, aberrant CBM function has been shown to play roles in diseases such as B-cell lymphoma and auto-immunity. Upon antigen receptor engagement, the CARD11 subunit is phosphorylated by protein kinase C (PKC), which activates its function by reducing interaction of its auto-inhibitory coiled coil domain to its CARD domain. The activated form CARD11 then can interact with BCL10 and facilitate its forming of large macromolecular filaments, providing a large scaffold for binding and activation of MALT1, which is the enzymatic paracaspase subunit of the CBM complex that results in further downstream activation of a variety of effector molecules.

Like other supramolecular organizing center (SMOC) mediated signaling transduction, such as toll-like receptor (TLR) triggering Myddosome, RIG-I like receptor sensing intracellular viral RNA and activating mitochondrial antiviral signaling protein (MAVS) filament formation, the BCL10 filament formation is also important for BCR/TCR signaling amplification and robust downstream NF-xB activation. BCL10 is composed of an N-terminal CARD domain, and a long C-terminal unstructured region containing a distal Ser and Thr rich region. Structure guided studies showed that BCL10 filament polymerizes in a unidirectional manner through CARD-CARD interactions, providing a surface for cooperative binding of MALT1 through its N-terminal Death Domain. Upon BCL10 filament binding, MALT1 is immediately dimerized and incorporates TRAF6 to form higher ordered assembly leading to all-or-none activation of downstream pathways including NF-xB and JNK. Binding to BCL10 also activates MALT1 paracaspase activity and cleavage of substrate proteins. BCL10 filament formation is dynamic in activated T lymphocytes and precisely regulated by disassembly and degradation through BCL10 K63 polyubiquitination and p62 dependent selective autophagy-lysosomal proteolysis system. Hence dynamic BCL10 filament turnover might be important to precisely tune its effect on downstream signaling pathways such as NF-xB.

Chronic active NF-xB signaling is a hallmark of the highly aggressive activated B cell-like diffuse large B-cell lymphomas (ABC-DLBCLs), due to somatic mutations of BCR and Toll-like receptors (TLR) subunits such as CD79b and MYD88, as well as activating mutations of CARD11 and amplifications of MALT1. Collectively these mutations induce chronic activation of the CBM complex to maintain robust and sustained NF-xB and other downstream pathway activation. The involvement of these signaling pathways in highly aggressive tumors has inspired development of targeted therapies disrupting oncogenic BCR/TLR activity. However, the position where mutations happen in the BCR pathway may be important for assigning potential precision therapy to patients. For example, mutations in the most upstream BCR proteins like CD79B confer sensitivity to BTK inhibitors, whereas downstream mutations like PLCγ2 and CARD11 confer resistance. Hence mechanistic study of oncogenic mutations is beneficial to guide targeted therapy in B cell lymphomas. Recent genomic sequencing studies in DLBCLs and other lymphomas have revealed recurrent and widely spread somatic mutations of BCL10, However, the functionality and mechanism of BCL10 mutations in DLBCL have not been studied. Whereas it is evident how mutations causing constitutive activation of CARD11 or increased abundance of MALT1 might result in enhanced CBM function, it is not immediately clear how these BCL10 mutations might function. Therefore, the structure and function of BCL10 mutations in DLBCL were explored by identifying distinct classes of mutant proteins with different biochemical effects and distinct impact on response to targeted therapies. These studies have implications for selecting targeted therapy agents for lymphoma precision therapy.

### Results

### BCL10 Mutations are Genetic Drivers and Occur in Two Broad Classes.

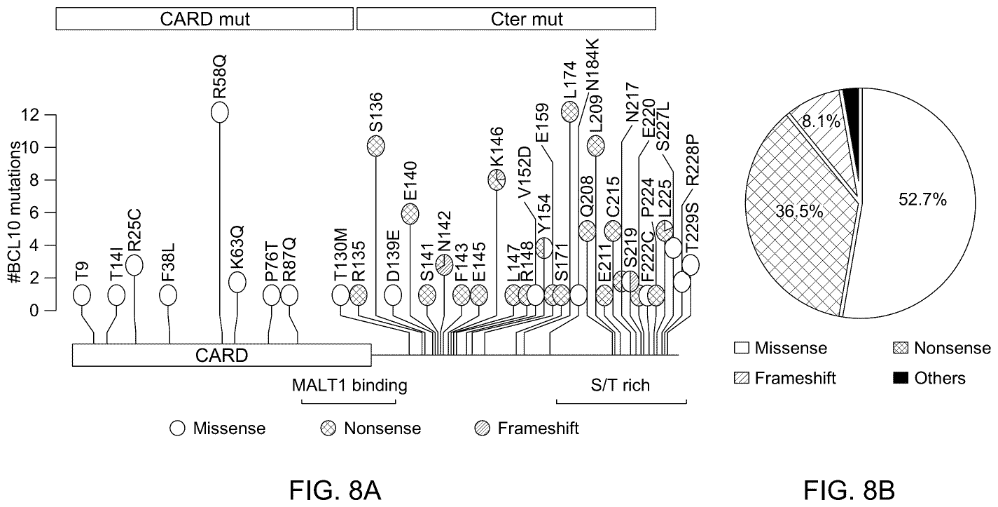

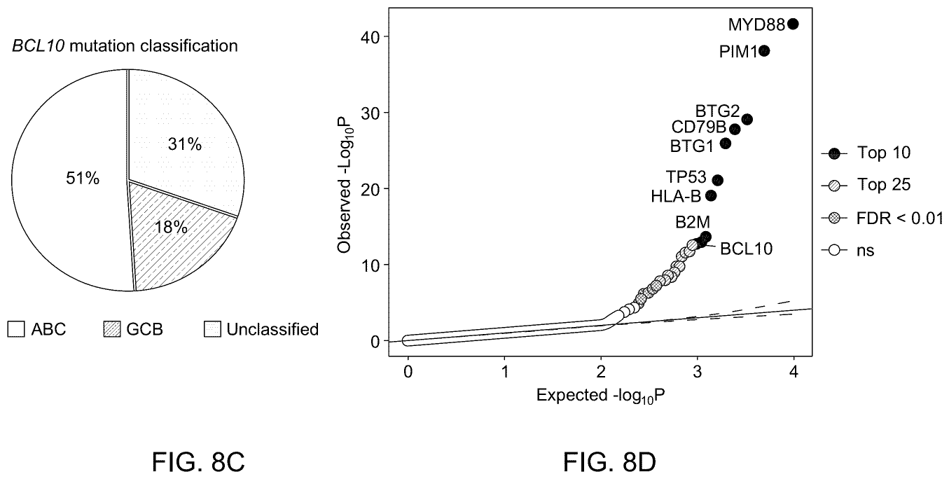

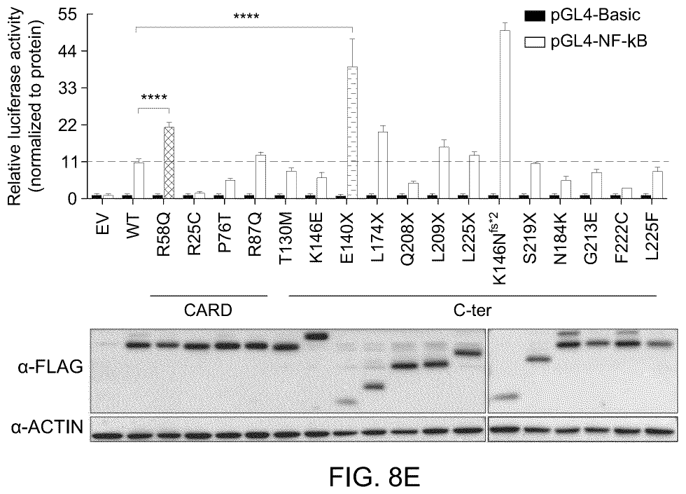

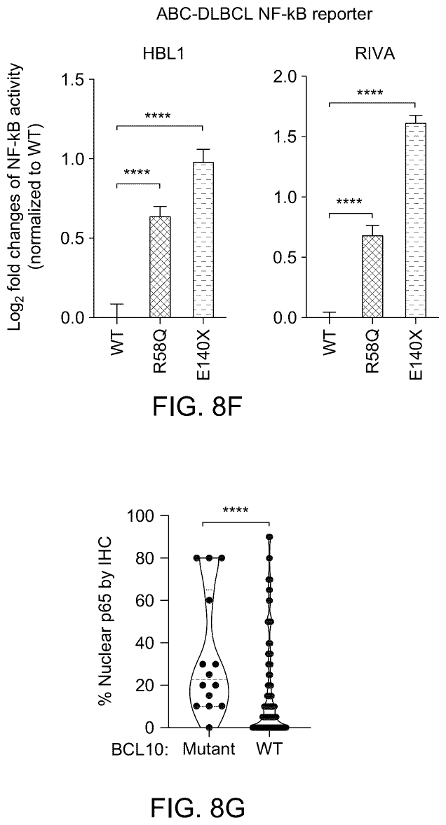

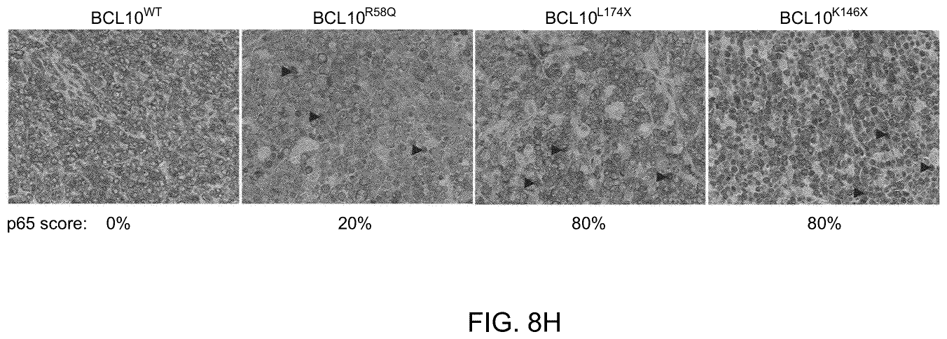

As a first approach to explore structure-function of BCL10 mutations DLBCL sequencing databases were merged (n=2255) and BCL10 mutant patients identified. BCL10 contains a structured CARD domain at its N-terminal half that mediates interaction with CARD11 and polymerization of BCL10 into fibrils. The C-terminal region is unstructured and contains serine and threonine residues targeted by post-translational modifications. BCL10 mutations affected both regions. Although mutations in the CARD were all missense mutations with a prominent hotspot at Arginine 58, in contrast a majority of those in the C-terminal region were truncating (nonsense or frameshift) mutations with a number of hotspot residues observed (FIG. 8A-B).

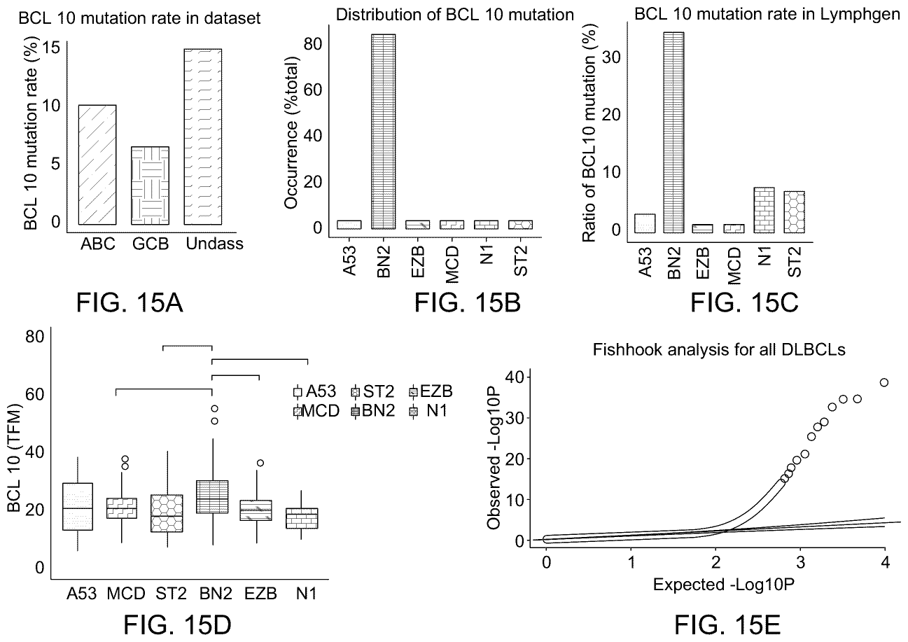

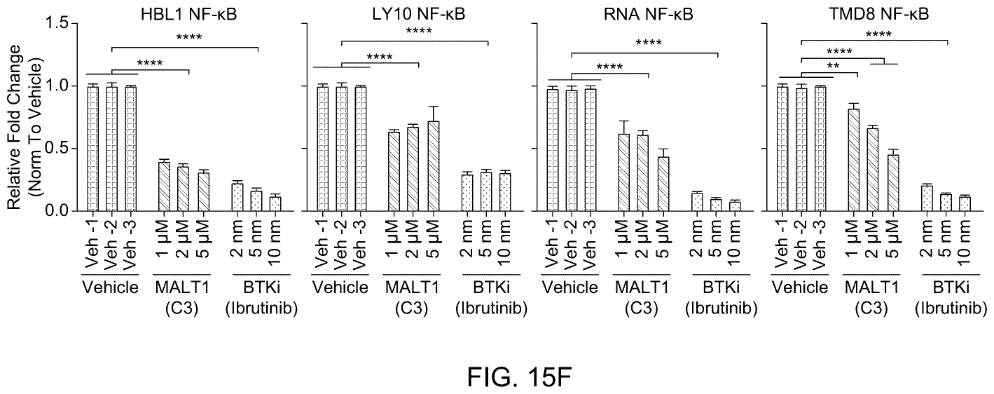

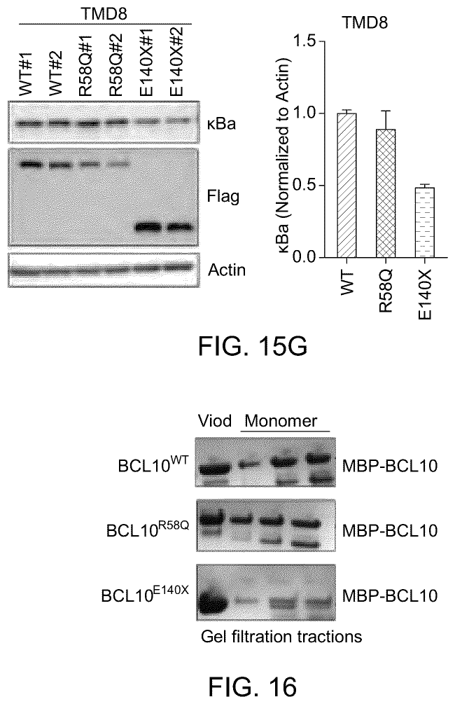

Examining BCL10 mutations in a cohort of patients with rigorous cell of origin and genetic cluster information, it was observed that 51% occurred in ABC-DLBCLs, 31% in unclassifiable cases, and 18% in GCB-DLBCLs (FIG. 8C). However, the incidence of BCL10 mutation was highest in the unclassifiable patients, followed by the ABC-DLBCLs (FIG. 15A). As reported previously using the LymphGen classification system, most BCL10 mutations were observed in the BN2 class of DLBCLs (FIG. 15B), which had the highest incidence of these lesions (35% of patients, FIG. 15C). BCL10 expression was also highest among the BN2 cases (FIG. 15D).

To determine whether BCL10 somatic mutations were likely to be robust genetic drivers of ABC-DLBCLs, a rigorous genomic co-variate “Fish-hook” analysis controlling for gene size, as well as GC B-cell gene expression profiles, activating promoter histone marks, chromatin accessibility profiles and others were performed. This analysis captured BCL10 as one of the top 10 driver mutations in ABC-DLBCL, along with genes such as MYD88, CD79B, PIM1 and TP53 (FDR<0.01, FIG. 8D). BCL10 mutations were still among the top 15 drivers when considering all DLBCLs (FIG. 15E).

Next, to survey whether the different classes of BCL10 mutations had a functional impact on NF-xB signaling, a panel of CARD and C-terminal mutants were expressed, as well as wild-type BCL10 together with an NF-xB luciferase reporter in 293T cells. As expected, WT BCL10 was able to induce NF-xB activity (FIG. 8E). Most of the CARD and C-terminal mutations also induced NF-xB activity. However, the hotspot missense mutant BCL10^{R58Q} showed significantly higher NF-xB activity compared to wildtype BCL10, as did the truncating mutants BCL10^{E140X} and BCL10^{K146Nfs*2 (FIG}. 8E).

Focusing the studies on representative CARD and C-terminal mutations, this markedly increased NF-κB reporter induction even occurred in ABC-DLBCL cells that already have chronic BCR activation including both MCD (HBL1) and BN2-DLBCL cells (RIVA) (FIG. 8F), also validating that this reporter reacts as expected to disruption of NF-κB signaling (FIG. 15F). Hence, both missense and truncating BCL10 mutations yield a significant gain of function effect on NF-κB activation. Using an additional MCD cell line TMD8, it was also noted reduction in IκBα abundance in cells expressing BCL10^{E140X} truncating mutant (FIG. 15G). Finally, to determine if such findings could be validated in primary human DLBCLs, immunohistochemistry staining of p65 was performed in a set of tissue microarrays containing biopsies from 298 genetically annotated DLBCL patients. It was found that BCL10 mutant DLBCLs manifested significantly increased p65 nuclear staining scores compared to BCL10^{WT} cases (Mann-Whitney p<0.0001, FIG. 8G). BCL10 truncating mutations associated with the highest nuclear p65 scores, whereas BCL10^{R58Q} CARD mutants less abundance of nuclear p65 that was still higher than in BCL10^{WT} cases (FIG. 811).

### BCL10 Polymerization is Greatly and Moderately Enhanced Respectively by E140X and R58Q Mutants.

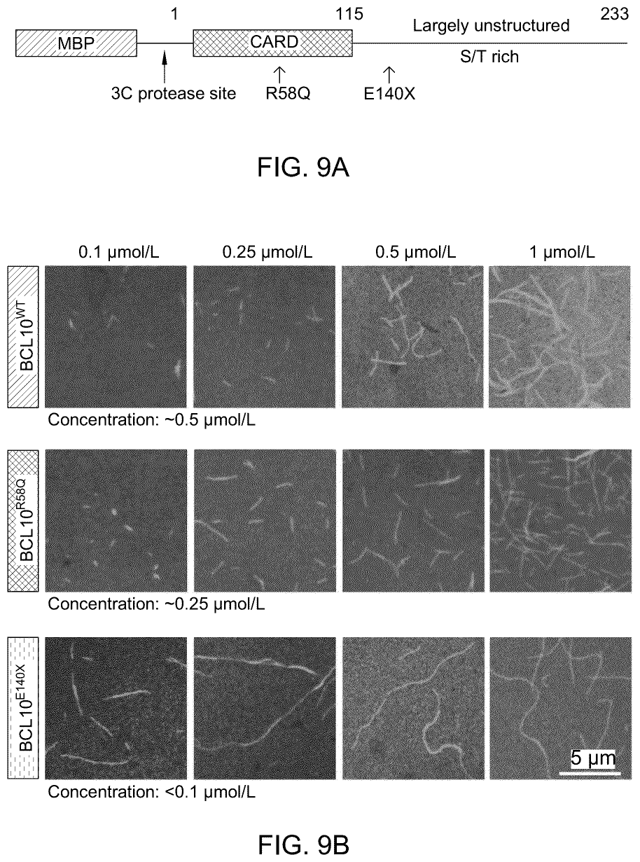

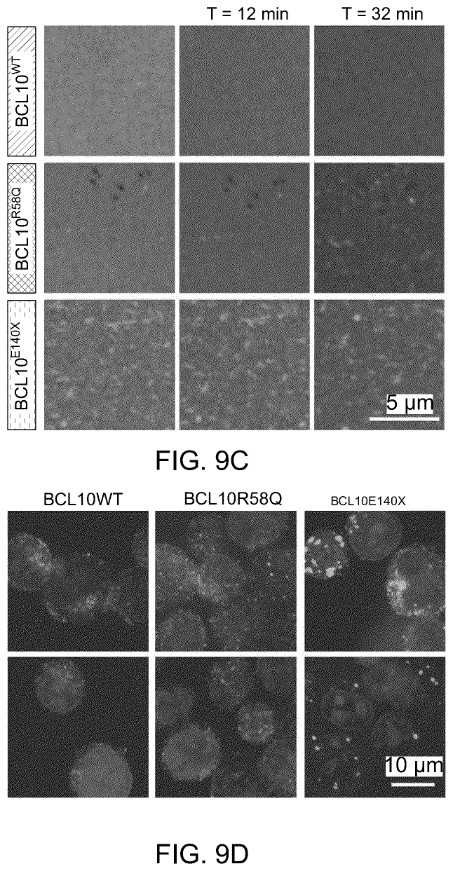

To determine the mechanism through which BCL10 mutants might confer a biochemical gain of function, full-length BCL10^{WT}, BCL10^{E140X} and BCL10^{R58Q} fused to a 3C protease-cleavable maltose binding protein (MBP) at the N-terminus to keep the proteins in a monomeric state were expressed and purified (FIG. 9A). The purified proteins were labeled with Alexa488 through cysteine residues in the unstructured C-terminal region. Spontaneous filament formation was then assessed by confocal fluorescence microscopy as a function of BCL10 concentration 4 h after mixing with the 3C protease to remove the MBP tag. Judging from these images, BCL10^{WT} had a concentration of polymerization at ˜0.5 M while BCL10^{R58Q} initiated filament formation at a somewhat lower concentration of ˜0.25 M (FIG. 9B). Strikingly, BCL10^{E140X} started to form filaments even at the lowest concentration tested of 0.1 μM, indicating greatly enhanced ability to polymerize spontaneously (FIG. 9B). Of note, even MBP-fused BCL10^{E140X}, in which MBP should suppress filament formation sterically, had a significant polymerized fraction during protein purification; in contrast, we did not observe this phenomenon for BCL10^{WT} or BCL10^{R58Q} (FIG. 16B). The kinetics of filament formation by these proteins was imaged at 1 M concentration, which is above the concentration of polymerization for WT and these mutant BCL10 proteins using time lapse confocal fluorescence microscopy. It was found that BCL10^{E140X} was already extensively polymerized at 2 min post-mixing with 3C protease, the earliest time point we could image (FIG. 9C). While filament formation kinetics of BCL10^{WT} and BCL10^{R58Q} were both slower than that of BCL10^{E140X}, BCL10^{R58Q} showed more apparent filament formation at 32 min after addition of the 3C protease than BCL10^{WT} (FIG. 9C), suggesting that BCL10^{E140X} and to a lesser extent BCL10^{R58Q} accelerated BCL10 polymerization threshold and kinetics.

Activated C8M complexes manifest as puncta when visualized through confocal microscopy of living cells. ABC-DLBCL cells (HBL1) engineered for constitutive expression of FLAG-tagged BCL10^{WT}, BCL10^{E140X} and BCL10^{R58Q}, respectively, were imaged. These experiments revealed large, striking aggregates of BCL10^{E140X}, in comparison with the much smaller puncta of BCL10^{WT} or BCL10^{R58Q} (FIG. 9D), supporting the in vitro findings using recombinant proteins. These aggregates are reminiscent of the large puncta of an oncogenic, gain-of-function CARD11 mutant suggesting that these aggregates represent active CBM complexes.

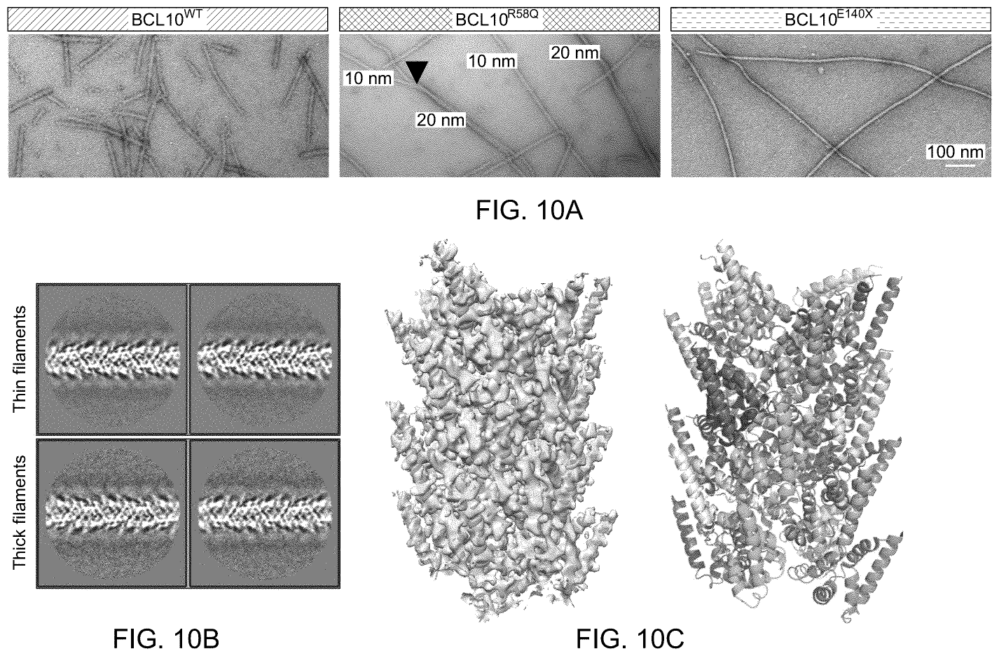

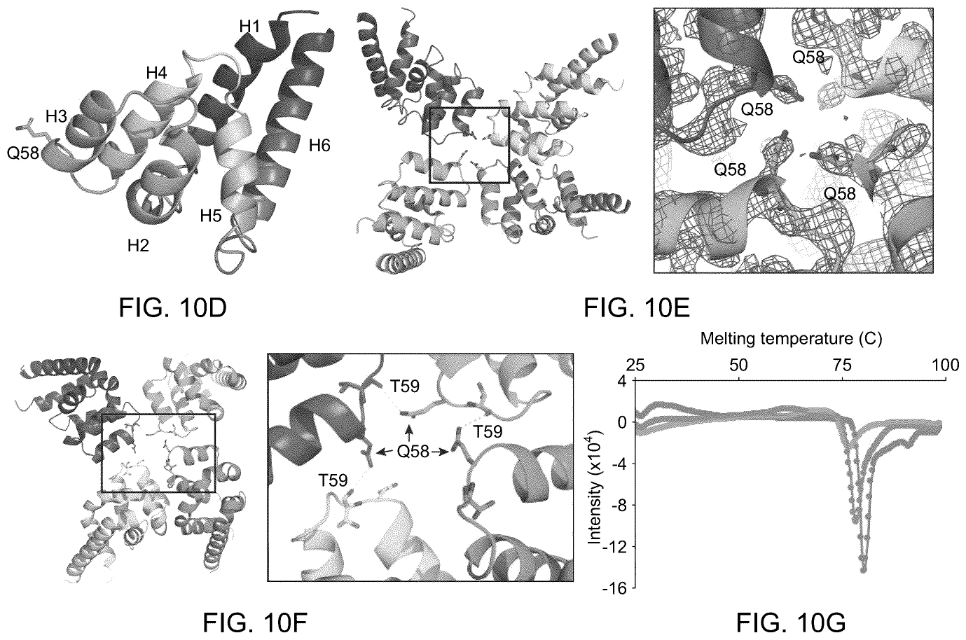

The R58Q mutant forms filaments with a glutamine ladder, enhanced stability and tendency to bundle. To better visualize these filaments, 3C protease-treatment induced filaments of BCL10w, BCL10^{R58Q} and BCL10^{E140X} were purified by collecting the void fraction (i.e., polymerized BCL10) from a Superose 6 gel filtration column, and imaged using scanning electron microscopy (EM) (FIG. 10A). Surprisingly, whereas BCL10^{WT} and BCL10^{E140X} formed the classical 10 nM filaments of CARDs, BCL10^{R58Q} formed both 10 nm filaments and thicker 20 nM filaments (FIG. 10A). Closer inspection of the nM filaments suggested that they are bundled 10 nM filaments because these thinner filaments were observed to merge into thicker filaments (FIG. 10A).

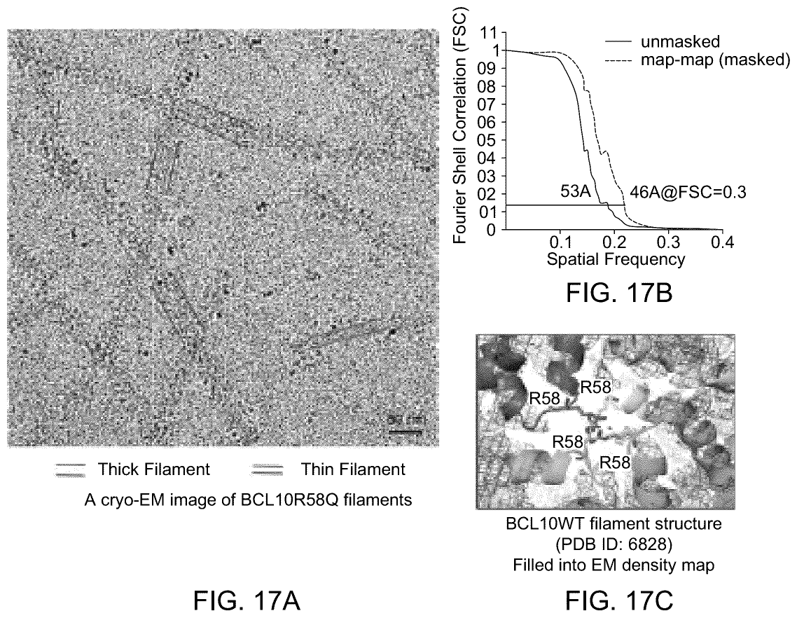

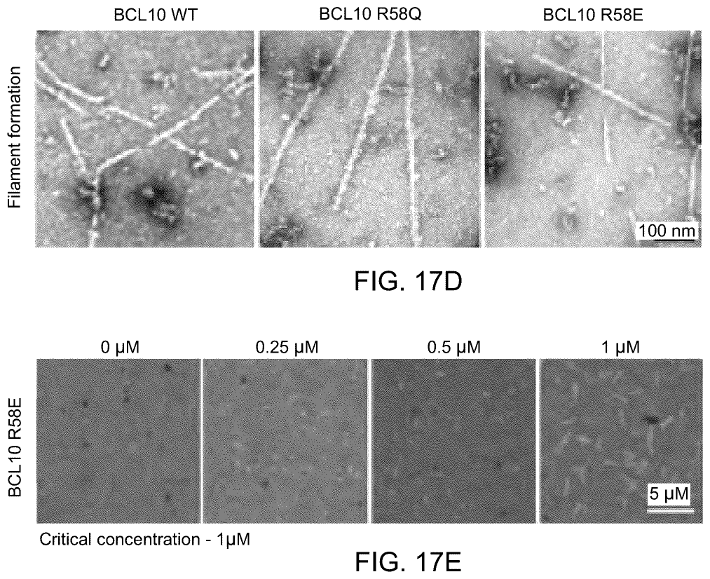

To determine if the BCL10^{R58Q} filaments are structurally different from BCL10^{WT} or BCL10^{E140X} filaments and how the bundling occurs, cryo-EM data was collected using an Arctica microscope operating at 200 keV and a K2 electron counting direct detection camera (FIG. 17A). Thin and thick filaments were manually selected from the cryo-EM micrographs and noted that 2D classification revealed average filaments of similar thickness (FIG. 10B). These data suggested that the association between filaments within the bundle is not specific so that only one thin filament within each thick filament could be aligned and the other filament was averaged out. Thus, the thick filaments consist of randomly bundled thin filaments (FIG. 10B).

Using 3D reconstruction, the cryo-EM structure of the BCL10^{R58Q} filament at 4.6 Å resolution was determined, assessed by gold-standard Fourier shell correlation (FSC) (FIG. 17B). The overall structure was similar to that of the BCL10T filament with only the CARD domain ordered (FIG. 10C). Q58 resides in helix 3 (H3) of the six-helical bundle fold of the CARD domain (FIG. 10C). Within the BCL10^{R58Q} filament structure, Q58 localizes near the center and its side chain density is well defined (FIG. 10E), which contrasts with the poor density of the equivalent wild type R58 in the BCL10^{WT} filament structure (FIG. 17C). While the resolution of the structure is limited, it is tempting to speculate that Q58 residues at the center of the filament formed stacks of glutamine residues, with direct and possibly water-mediated interactions. Indeed in the center of the filament, we found that Q58 side chain (NE2 atom) of one protomer in the filament forms a potential hydrogen bond with the carbonyl oxygen of T59 of the next protomer in the helical filament (FIG. 10F). To further demonstrate the potential role of the hydrogen bonding network in Bcl10 filament assembly in the R58Q mutant, the R58E mutant which does not have the NE2 atom (e.g., Q58 side chain) for hydrogen bond formation was generated and its biochemical and biophysical properties characterized. There was no enhancement of filament formation for R58E (FIG. 17D) and the concentration for R58E polymerization is ˜1 μM was even higher than WT BCL10 (FIG. 17E), confirming that the hydrogen bond formed by Q58 is important for its filament formation.

These Q58-mediated interactions prompted us to ask whether there might be a difference in the stability of the BCL10^{R58Q} filament in comparison to BCL10^{WT} and BCL10^{E140X} filaments. To assess this possibility, thermal melt assays were performed on purified BCL10^{WT}, BCL10^{R58Q} and BCL10^{E140X} filaments, which revealed that BCL10^{R58Q}, and to a lesser degree BCL10^{E140X}, yielded more stable filaments, with thermal melting temperatures of 80.8° C. and 78.8° C. respectively, in comparison with 76.6° C. for the BCL10^{WT} (FIG. 3G). Thus, while BCL10^{E140X} shows enhanced polymerization, BCL10^{R58Q} forms more stable filaments, which may explain its tendency to bundle.

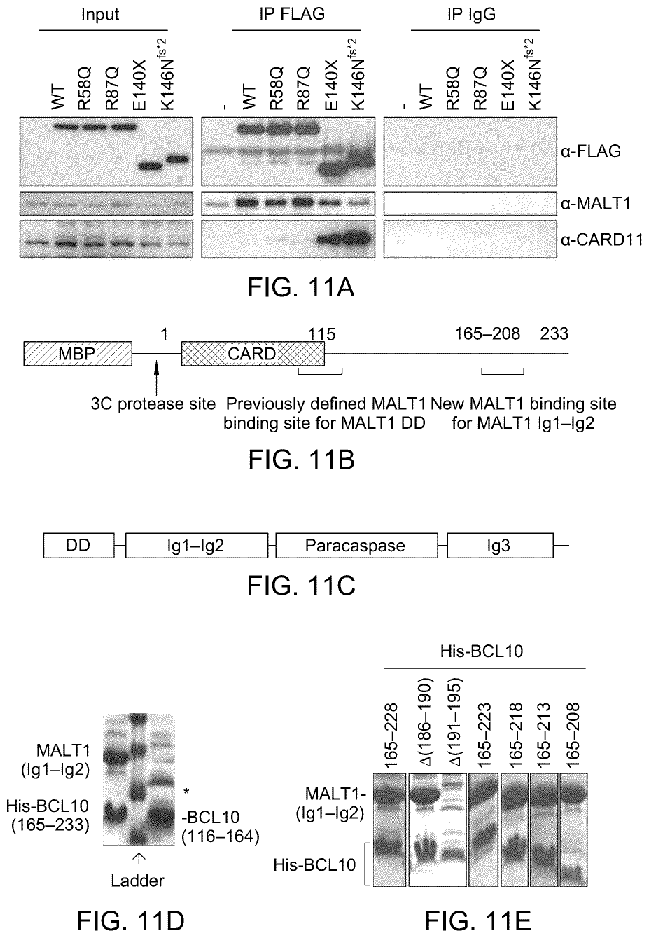

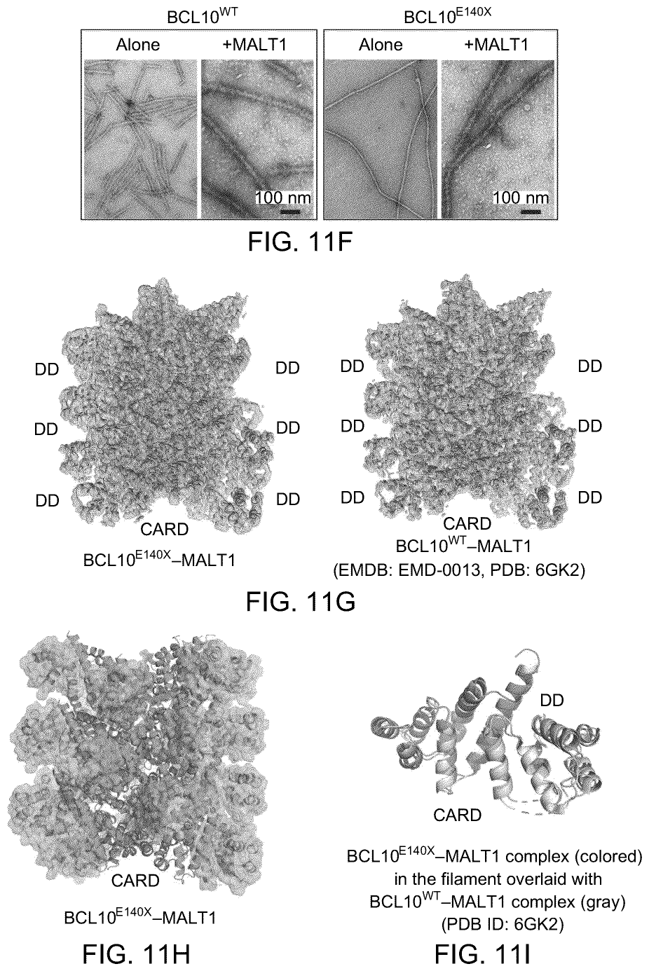

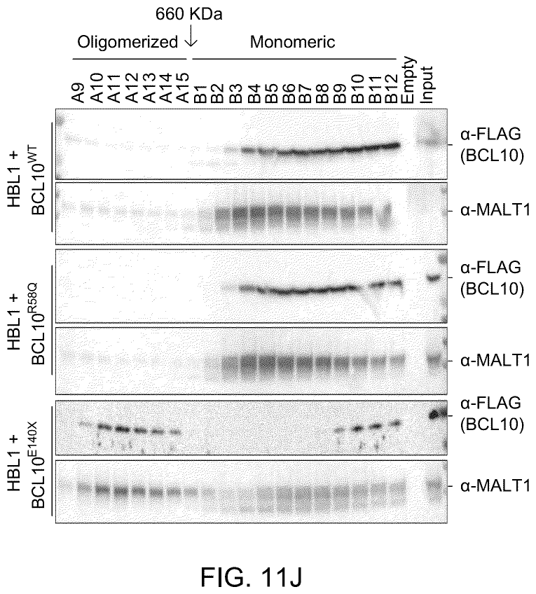

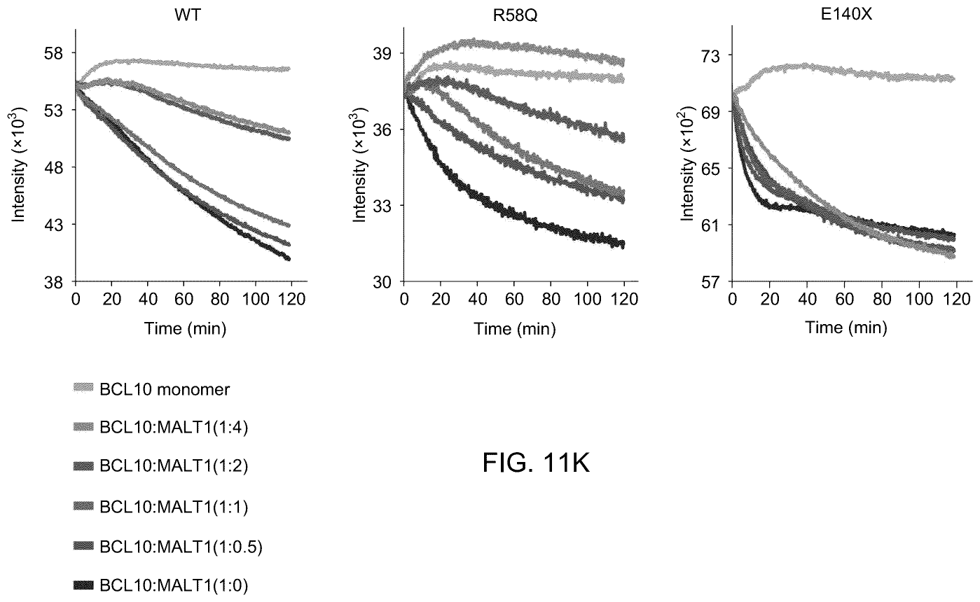

Loss of basal MALT1 binding promotes spontaneous polymerization of BCL10^{E140X}. To investigate how the truncation mutants might affect CBM complex formation, Flag-tagged WT and mutant forms of BCL10 were expressed in Raji cells, which lack constitutive BCR signaling. Performing anti-Flag co-immunoprecipitations, equivalent enrichment for MALT1 in WT and BCL10^{R58Q} as well as in another CARD missense mutant BCL10^{R58Q} was observed. In contrast, there was less binding of MALT1 to BCL10^{E140X}, as well as the similar BCL10^{K146Nfs*2} truncation mutant (FIG. 11A). While BCL10 CARD mutants interacted with CARD11 marginally better than BCL10w, the C-terminal mutants manifested much greater CARD11 interaction, which is likely due to the increased spontaneous polymerization of truncated BCL10, since BCL10 polymers were shown to enhance interaction with CARD11. The observed weaker recruitment of MALT1 by BCL10 truncation mutants was surprising as previous studies have mapped BCL10 CARD as MALT1 interacting domain (FIG. 11B) shown by mutagenesis and cryo-EM structure of the BCL10-MALT1 filamentous complex.

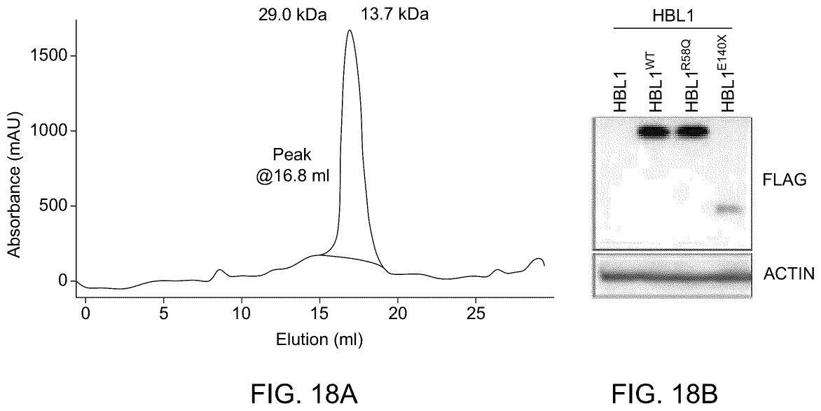

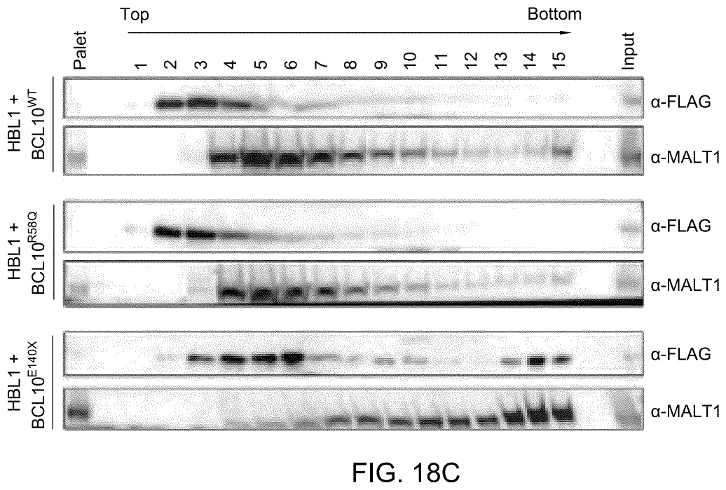

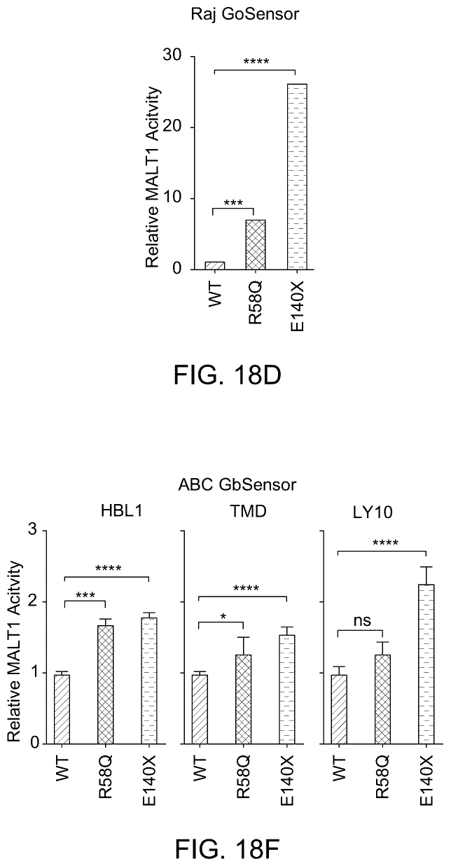

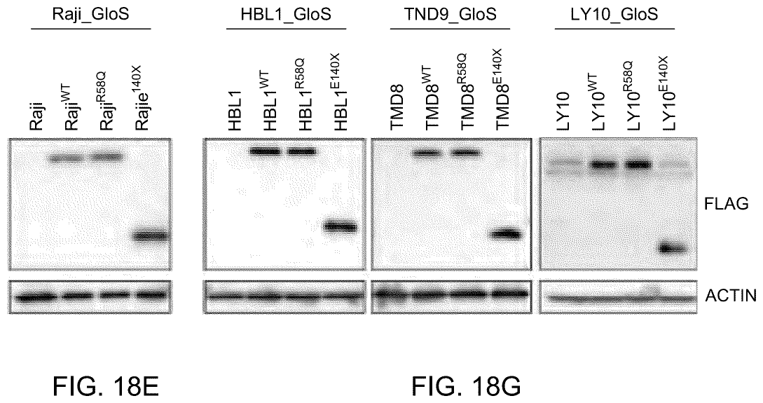

MALT1 has multiple domains (FIG. 11A) and in the reported cryo-EM structure, only the MALT1 death domain (DD) was ordered and interacted with the BCL10 CARD (residues 1-115), whereas the MALT1 immunoglobulin-like domains (Ig1-Ig2) and the paracaspase domain were not visible. Given that a previous mapping study suggested that the Ig1-Ig2 domains of MALT1 also interact with BCL10, it was unclear if there were additional MALT1-binding sites on BCL10 at its largely unstructured C-terminus. The C-terminal region was divided into two halves, and it was found that the second half (residues 165-233), but not the first half (residues 116-164), pulled down the Ig1-lg2 construct of MALT1 when co-expressed in E. coli shown by Coomassie blue-stained SOS-PAGE gel (FIG. 11D). Further truncations of the BCL10 165-233 fragment showed that a construct containing residues 165-208 was sufficient to pull down MALT1 Ig1-Ig2 (FIG. 11E), confirming this second MALT1-binding site (FIG. 11B). Of note, unlike the interaction between BCL10 CARD and MALT1 DD in the filamentous form, the interaction between BCL10 C-terminal region and MALT1 Ig1-Ig2 is monomeric as assessed by gel filtration chromatography of the complex (FIG. 18).

Given that this new MALT1 binding domain is deleted from BCL10^{E140X} we next explored whether there was any impairment in MALT1 recruitment to BCL10 filaments, by generating BCL10^{WT} and BCL10^{E140X} filaments in vitro and incubating them with purified, full length MALT1 followed by negative staining EM. These experiments showed equivalent patterns of MALT1 decorating the surface of WT and BCL10^{E140X} filaments, suggesting that MALT1 recruitment was intact in each case (FIG. 11F). However, given that this analysis does not have sufficient resolution to show whether MALT1 distribution on BCL10 polymers was altered, we next collected cryo-EM data on a Titan Krios microscope operating ats 300 keV and equipped with a Falcon II direct electron detector, and determined the cryo-EM structure of BCL10^{E140X} filaments in complex with MALT1 at 4.3 Å resolution. The structure of the BCL10^{E140X} filament with MALT1 was highly similar to that of the BCL10^{WT} filament with MALT1 at 4.9 Å resolution, in which the DDS of MALT1 bind the CARD of BCL10 and decorate the outside of the core CARD filament (FIG. 11G-I). The conserved structure confirmed that the truncation did not affect the association of BCL10 filaments with MALT1, but that BCL10^{E140X} is nonetheless defective in interacting with monomeric MALT1.

To further investigate these associations, gel filtration analysis was performed from lysates of ABC-DLBCL cells expressing FLAG-tagged BCL10^{WT}, BCL10^{R58Q} and BCL10^{E140X}, respectively (FIG. 18B). Cell fractionation of these lysates revealed a relatively small proportion of BCL10^{WT} or BCL^{R58Q} in high molecular weight fractions corresponding to filaments, along with a small fraction of MALT1, whereas most BCL10 and MALT1 proteins were in low molecular weight fractions (FIG. 11J). In marked contrast, BCL10^{E140X} was present at higher abundance in high molecular weight fractions, with corresponding enrichment of MALT1 (FIG. 11J). Reciprocally, there was reduced abundance of BCL10^{E140X} and a notable reduction of MALT1 in the lower molecular weight complexes. Similar findings were observed by performing sucrose gradient experiments (FIG. 18C).

The association between enhanced polymerization and lack of MALT1 monomeric interaction of BCL10^{E140X} prompted the hypothesis that MALT1 binding to the C-terminal region of BCL10 might inhibit BCL10 polymerization. To investigate whether this was the case Alexa488-labeled, MBP-fused BCL10^{WT}, BCL10^{R58Q} or BCL10^{E140X} were incubated with increasing concentrations of purified MALT1, treated the reactions with C3 protease to remove the MBP moiety, and monitored polymerization kinetics using fluorescence quenching (FIG. 11K). For BCL10^{WT} and BCL^{R58Q}, increasing doses of MALT1 suppressed BCL10^{E140X} filament formation in a dose dependent manner. By contrast, there was little suppression of BCL10^{E140X} polymerization by MALT1 at any dose. Taken together these data suggest that BCL10^{E140X}, and likely other similar truncation mutations, favor BCL10 polymerization in cells not only by reducing its intrinsic concentration threshold (FIG. 9B-C), but also by abrogating a novel MALT1 inhibitory effect mediated through interaction with the novel BCL10 C-terminal region binding site. By the same token loss of C-terminal tail binding would increase the pool of MALT1 that is available to bind to BCL10 polymers, suggesting that the end result would be potent enhancement of BCL10 filament formation and MALT1 activity.

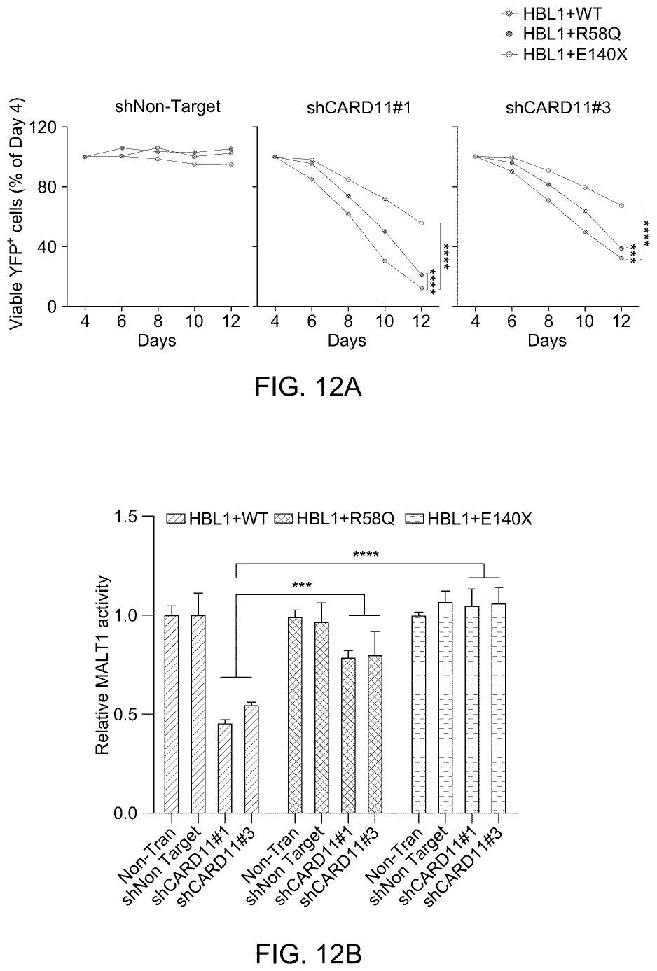

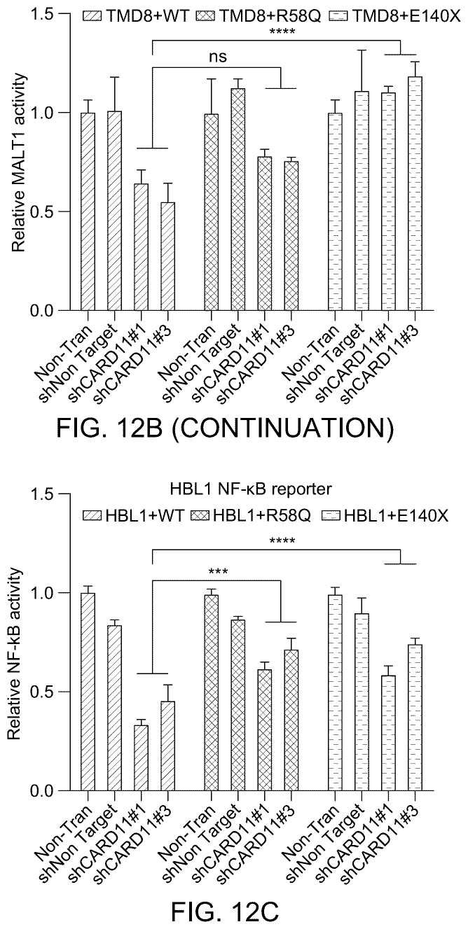

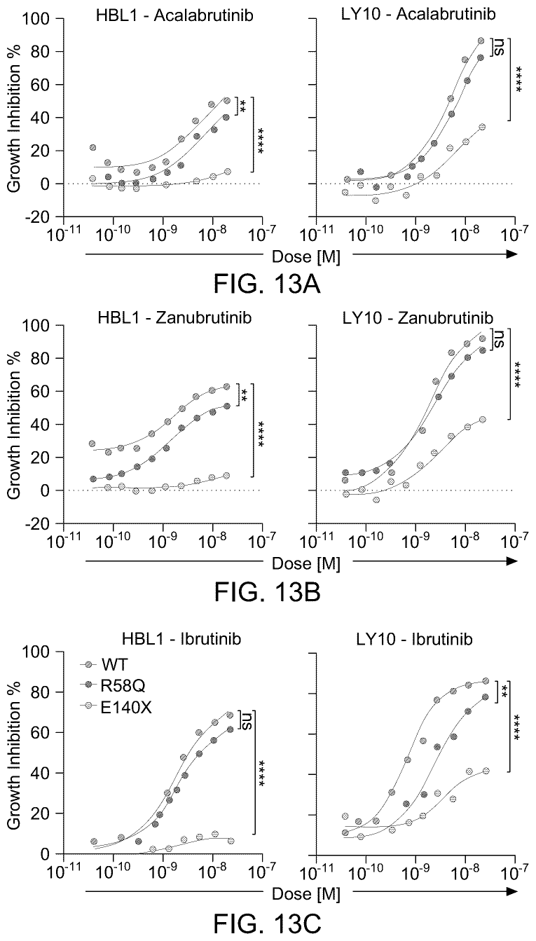

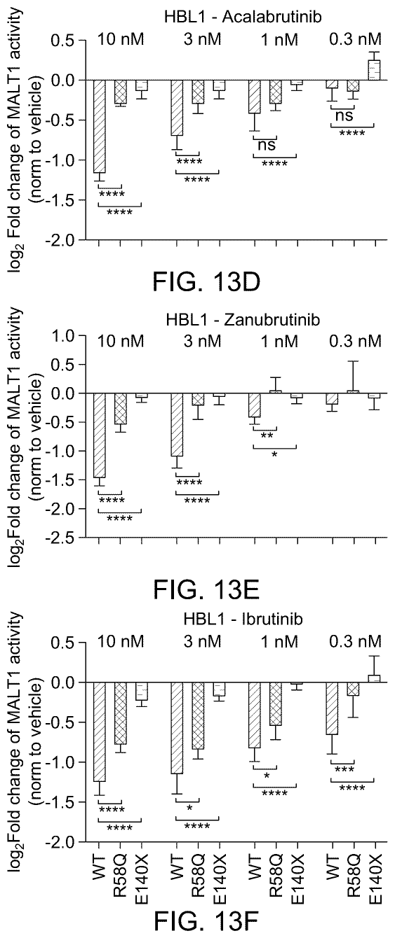

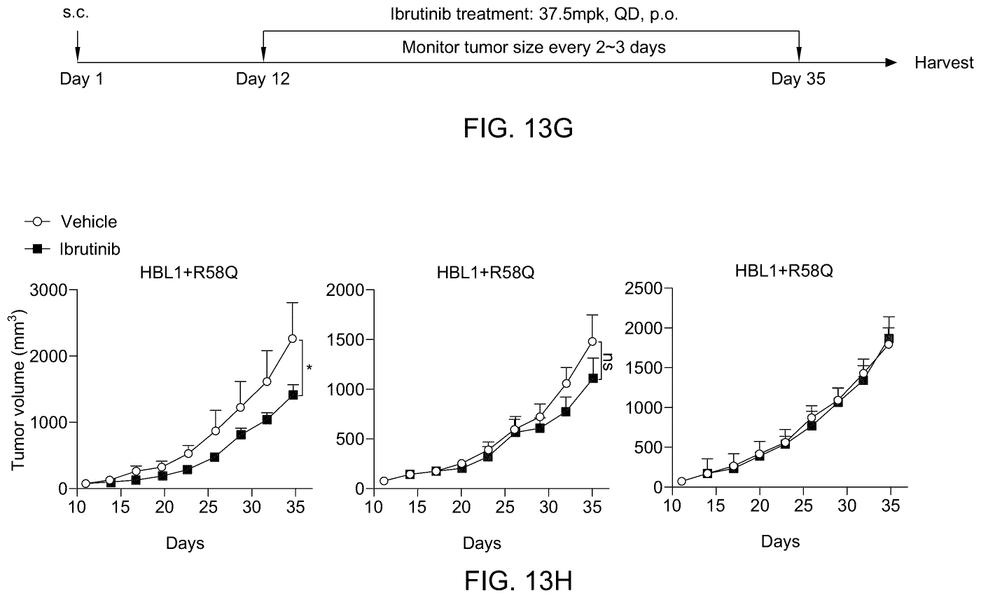

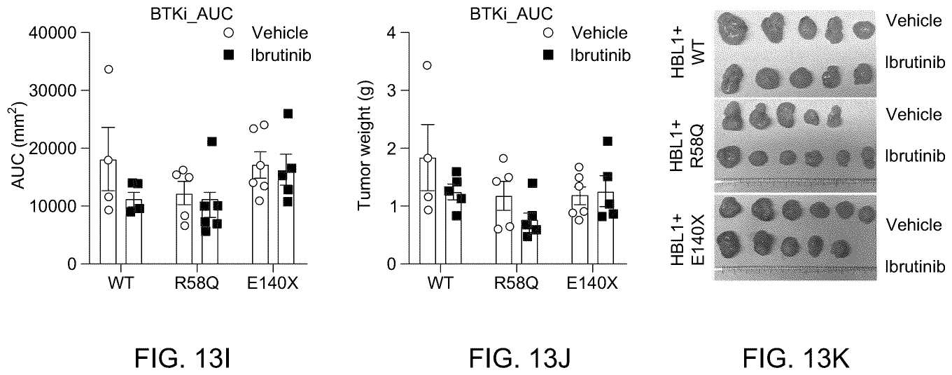

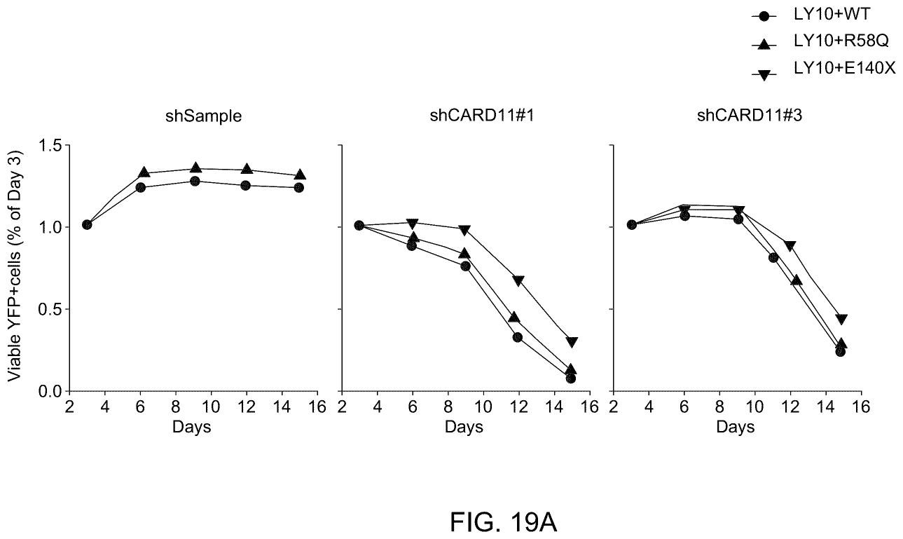

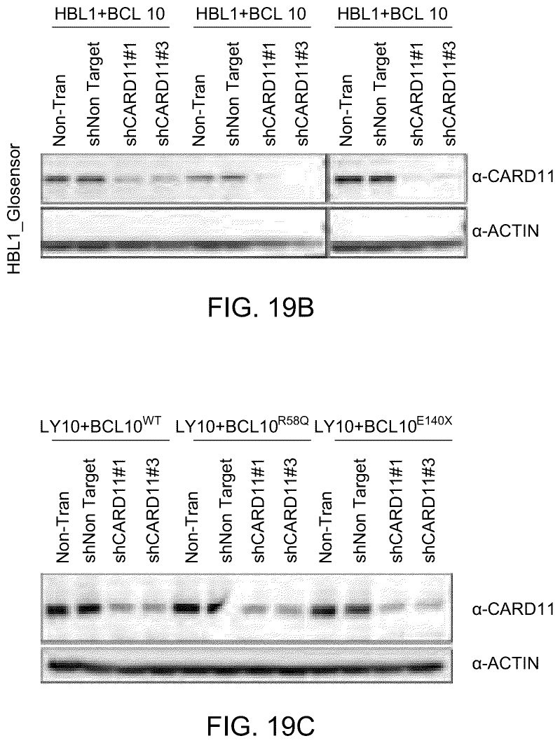

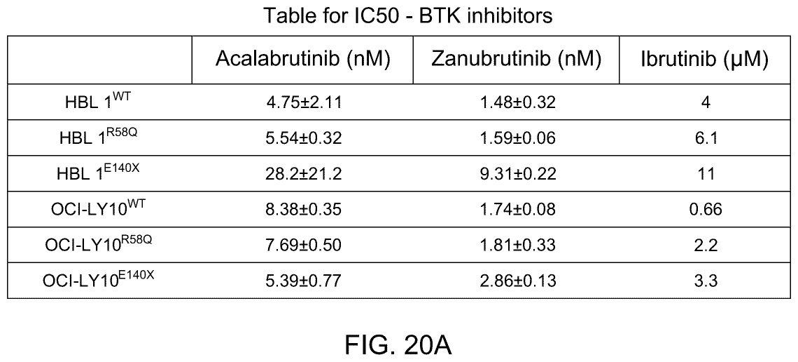

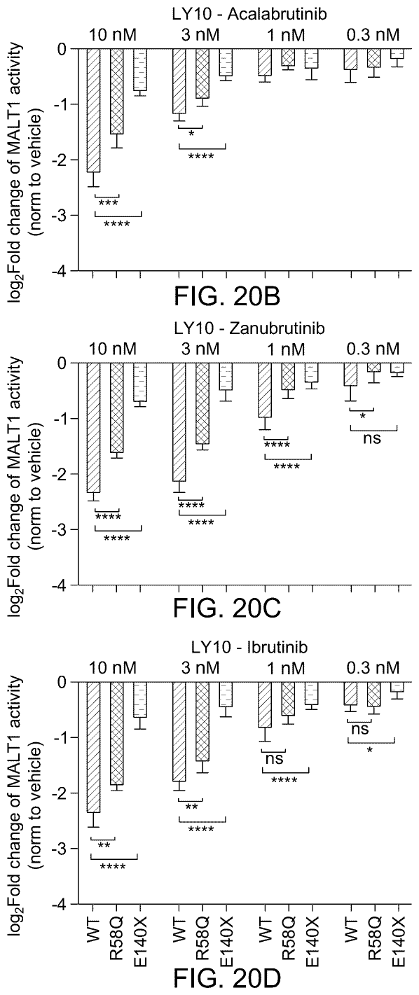

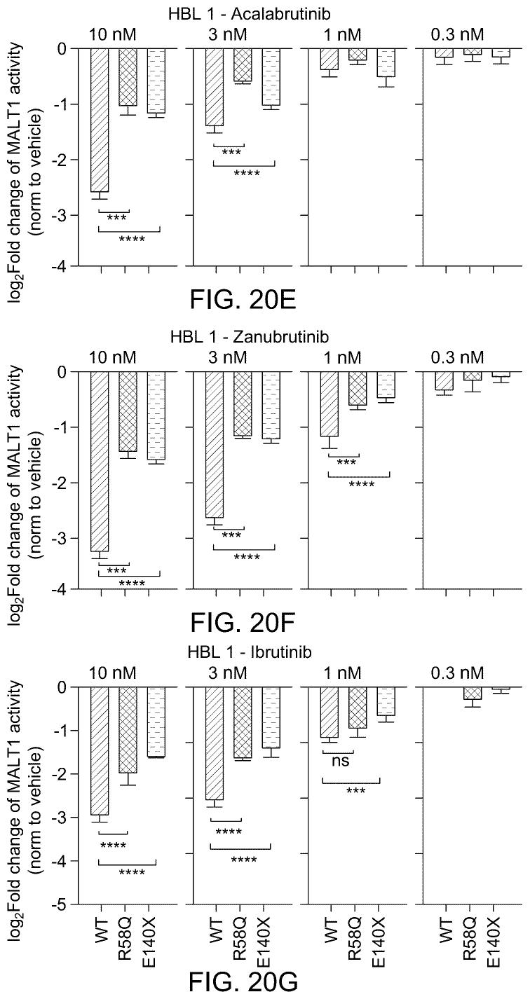

Differential activation of MALT1 by BCL10^{E140X} vs BCL10^{R58Q}. Because MALT1 dimerization on BCL10 filaments activates its proteolytic function, it was wondered whether skewing of MALT1 cellular pools towards the BCL10 polymer bound state in the BCL10^{E140X} setting might lead to higher cellular levels of MALT1 activity. The effect of BCL10 mutants on MALT1 activity within cells in the absence of basal BCR signaling was investigated. For this, MALT1 enzymatic reporter assays were performed using a GloSensor protein construct engineered with a specific MALT1 cleavage site stably transduced in Raji cells expressing BCL10^{WT}, BCL10^{E140X} or BCL10^{R58Q}(FIG. 18E). Significantly greater MALT1 enzymatic activation in BCL10^{E140X} cells was observed compared to both BCL10^{WT} or BCL10^{R58Q} (FIG. 18D). There was also greater MALT1 activity when comparing BCL10R to BCL10^{WT} albeit to lesser extent, consistent with the slightly enhanced polymerization kinetics of BCL10^{R58Q}. Finally, similar MALT1 protease reporter assays were performed in ABC-DLBCL cell lines expressing BCL10^{WT}, BCL10^{E140X} or BCL10^{R58Q}, where there is constitutive activation of signaling to the CBM complex. Again E140X generally yielded the strongest MALT1 activation (FIG. 18F-G), and BCL10^{R58Q} generally yielded greater MALT1 activity than WT BCL10. Collectively, these data indicate that both BCL10^{R58Q} and especially BCL10^{E140X}, through distinct mechanisms, lead to aberrantly increased MALT1 activity.
BCL10^{E140X} confers reduced dependency on CARD11 for CBM activation. Normally, active CARD11 is required to nucleate the formation of BCL10 filaments. However, it was wondered whether the requirement for CARD11 might be diminished in ABC-DLBCL cells expressing BCL10^{E140X}, given its greater tendency to polymerize and loss of MALT1 inhibitory interactions. CARD11 shRNA knockdown experiments were performed in isogenic ABC-DLBCL cells expressing BCL10^{WT}, BCL10^{R58Q} and BCL10^{E140X} CARD11 depletion is known to cause proliferation arrest of ABC-DLBCL cells and accordingly it was observed significant growth suppression induced by CARD11 knockdown in the presence of wild type BCL10 (FIG. 12A and FIG. 19A-C). However, this effect was significantly blunted in the presence of BCL10^{E140X} and to a lesser extent by BCL10^{R58Q}. To determine how this effect might relate to CBM complex function, the impact of CARD11 knockdown on MALT1 activity was tested using GloSensor reporter assays. MALT1 activity was highly impaired after CARD11 knockdown in the presence of WT BCL10, whereas this effect was completely rescued in BCL10^{E140X} cells and partially rescued by BCL^{R58Q}(FIG. 12B). CARD11 knockdown also reduced NF-κB reporter activation in BCL10^{WT} ABC-DLBCL cells, an effect that was blunted in CARD11 depleted in ABC-DLBCL cells expressing BCL10^{R58Q} and BCL10^{E140X} (FIG. 12C). Hence an additional perturbation explaining BCL10^{E140X} activation of MALT1 may link to its reduced requirement for CARD11 to induce filament formation, consistent with the data showing markedly greater activity in unstimulated B-cells. BCL10^{R58Q} which does not polymerize as readily as BCL10^{E140X} and is still inhibited by MALT1 monomers retains a greater degree of CARD11 dependency.
BCL10^{R58Q} and BCL10^{E140X} confer distinct levels of resistance to ibrutinib BTK inhibitors have emerged as a precision therapy modality for ABC-DLBCLs. However, lymphoma cells with inherent or acquired mutations in activating proteins downstream of BTK (e.g., CARD11 mutations that induce potent MALT1 activation) are often resistant to such treatments. Given the distinct functional profiles, CARD11 dependencies, and MALT1 activation effects of BCL10 CARD and truncation mutants, it was wondered whether and to what extent they might confer BTK inhibitor resistance. The isogenic ABC-DLBCL cells expressing BCL10^{WT}, BCL10^{R58Q} and BCL10^{E140X} proteins were treated with escalating doses of three chemically distinct covalent BTK inhibitors: ibrutinib, acalabrutinib or zanubrutinib, and tested their proliferation rates using an ATP fluorescence assay after 96 hours of drug exposure. BCL10^{R59Q} conferred at least a modest and often significant reduction in response to these drugs (FIG. 13A-C. FIG. 20A). In contrast, BCL10^{E140X} conferred far more dramatic resistance in almost all cases.

All three BTK inhibitors yielded potent and dose dependent suppression of MALT1 activity in BCL10^{WT} ABC-DLBCL cells (FIG. 13D-F, FIG. 20B-D). However, isogenic BCL10^{R58Q} and BCL1^{E140X} ABC-DLBCL cells manifested significantly less impact on MALT1, especially in the case of BCL10^{E140X}. Analyzing the further downstream impact of the BTK inhibitors on NF-κB reporter activity revealed significant impairment in BCL10^{WT}, which was significantly blunted in the presence of BCL10^{R58Q} and BCL10^{E140X} (FIG. 20E-G). Finally, ibrutinib (37mpk, oral gavage, Q.D.) was administered to mice bearing BCL10w, BCL10^{R58Q} and BCL10^{E140X} expressing ABC-DLBCL xenografts (FIG. 13G). lbrutinib yielded the expected growth suppression of BCLIO ABC-DLBCL tumors, but had no significant anti-tumor effect against the two mutant forms, which was most clearly evident in the case BCL10^{E140X} (FIG. 13H-K). Collectively, BCL^{R58Q} and BCL10^{E140X} confer distinct levels of resistance to BTK inhibition, consistent with their different mechanisms of action and impact on MALT1 activation.

BCL10 Truncating Mutant Lymphomas are Hypersensitive to MALT1 Protease inhibitor.

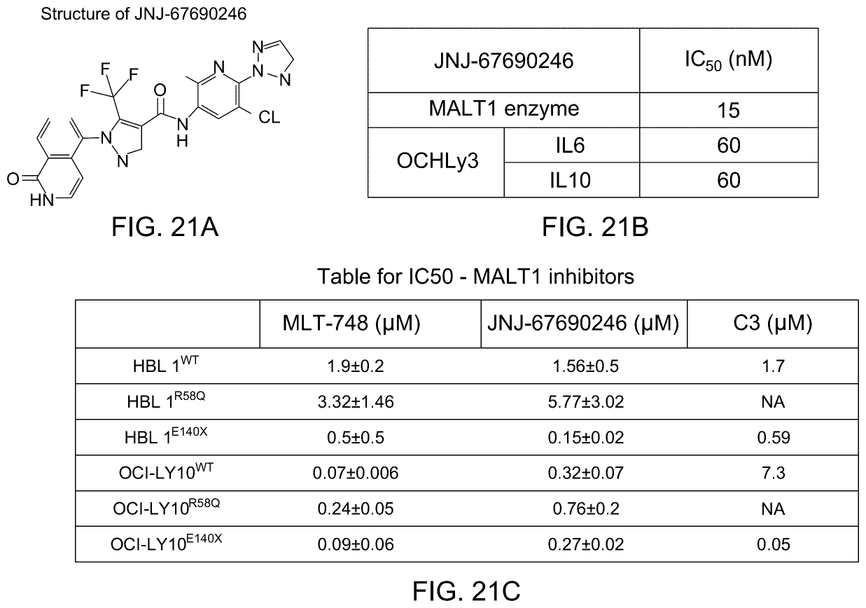

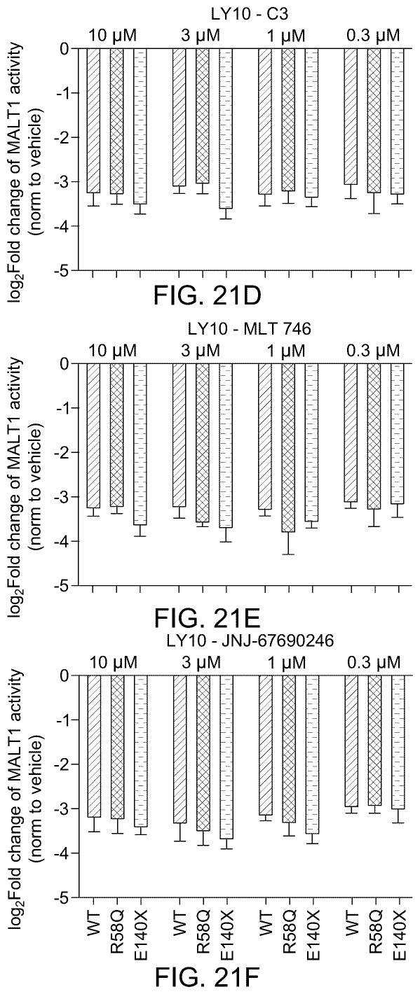

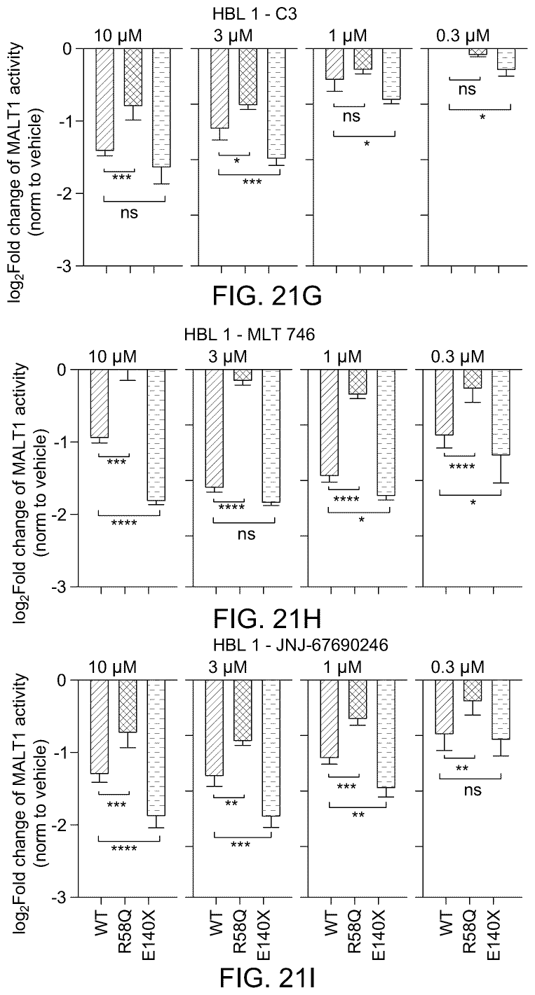

The fact that BCL10 mutants drive potent MALT1 activation even in the absence of CARD11 and confer reduced response to ibrutinib led to the hypothesis that these cells might be especially dependent on MALT1 and hence highly responsive to MALT1 inhibitors. To explore this question, the impact of three chemically and mechanistically distinct MALT1 inhibitors were tested against the set of isogenic ABC-DLBCL cells. These included: C3, a potent and specific compound that covalently inactivates the MALT1 catalytic pocket; MLT-748, a reversible allosteric compound that binds MALT1 Trp580 side chain thus to lock protease inactive and JNJ-67690246, an allosteric MALT1 inhibitor (FIG. 21A). JNJ-67690246 potently inhibits MALT1 enzymatic activity (IC50 =15 nM) in biochemical assays and cytokine secretion of 1L6/10 (IC50=60 nM) in OCI-Ly3 cellular assays (FIG. 21B).

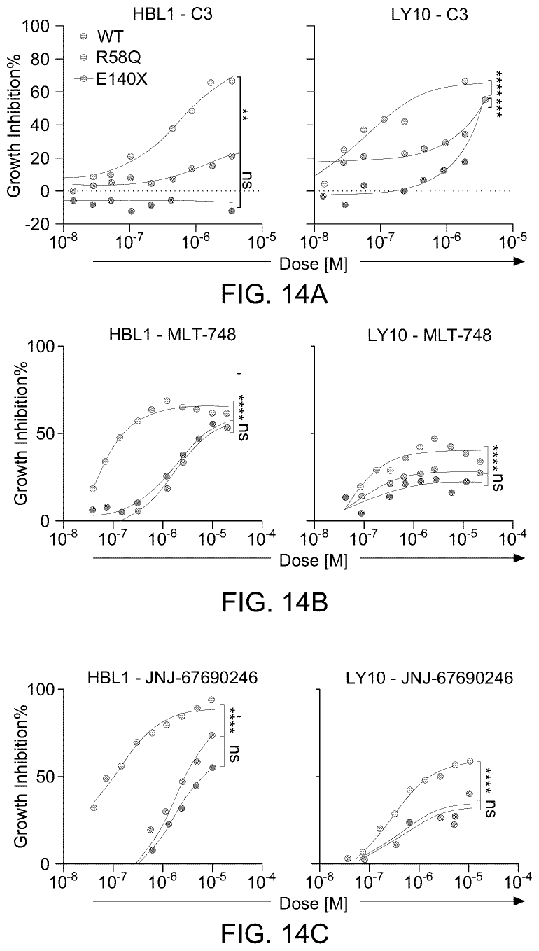

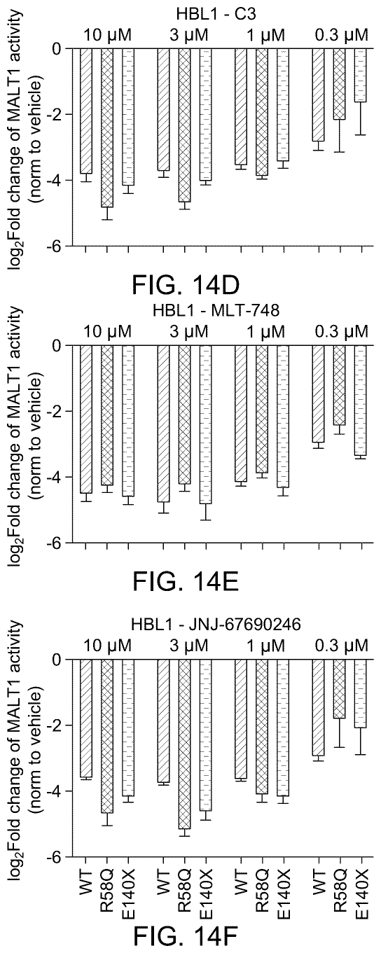

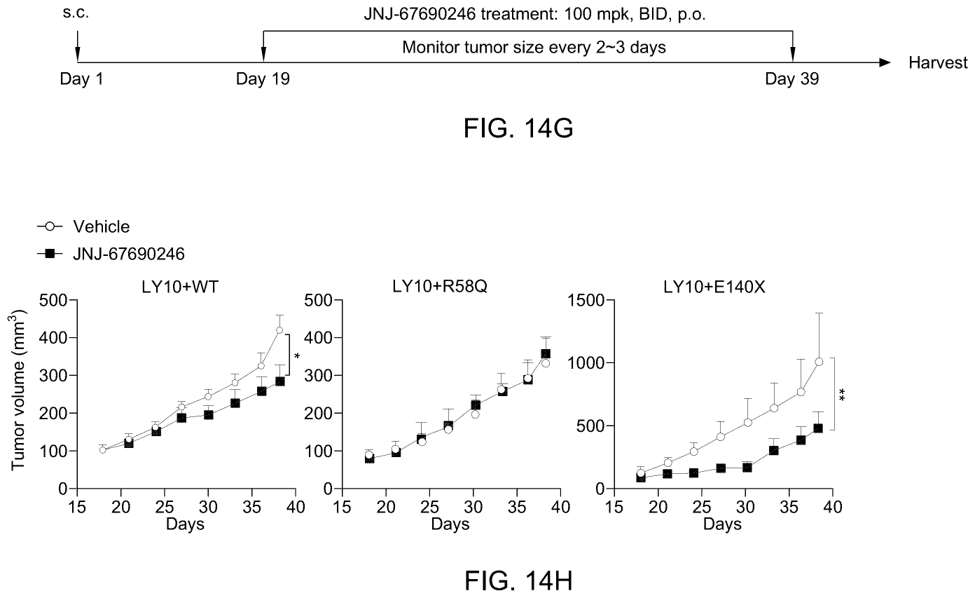

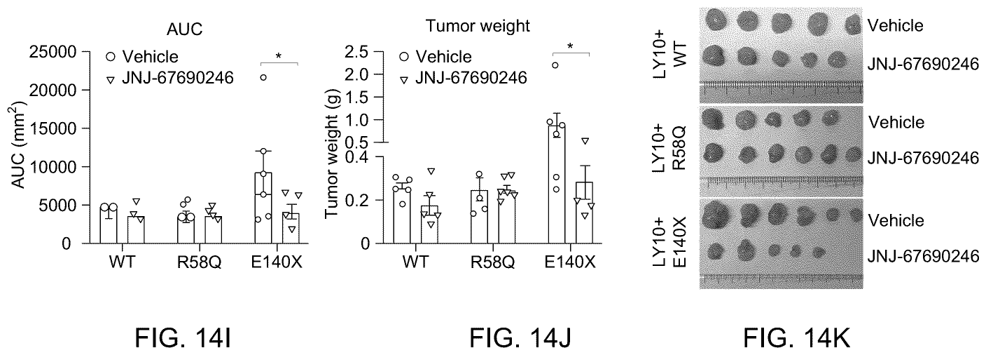

Isogenic BCL10^{WT}, BCL10^{R58Q} and BCL10^{E140X} ABC-DLBCLs were exposed to increasing concentrations of each of these compounds for 96 h, revealing striking differences in the response profiles of BCL10^{E140X} vs BCL10^{WT} and BCL10^{R58Q} (FIG. 14A-C, FIG. 21C). Both WT and BCL15% were generally sensitive to the allosteric inhibitors, whereas BCL10^{R58Q} cells were less sensitive than WT to C3. In marked contrast, BCL10^{E140X} manifested significantly greater response to all three MALT1 inhibitors. This differential effect was not due to variation in the degree of MALT1 inhibition, since MALT1 activity was equivalently suppressed by all three drugs in BCL10^{WT}, BCL10^{R58Q} and BCL10^{E140X} ABC-DLBCL cells (FIG. 14D-F, FIG. 21D-F). Analysis of NF-κB reporter activity showed significantly greater impairment in MALT1 inhibitor treated BCL10^{E1} OX cells as compared to either BCL10^{WT} or BCL10^{R58Q} (FIG. 21G-I), with the latter even showing less impairment of the NF-κB activity than in WT cells, suggesting that some other pathway may be maintaining NF-κB and hence conferring less dependency on MALT1 than in BCL10^{E140X} cells. Finally, BCL10^{WT}, BCL10^{R58Q} and BCL10^{E140X} ABC-DLBCL xenografts were treated with JNJ-67690246 in vivo, using the OCI-Ly10 cell line which is generally less sensitive to MALT1 inhibition, for greater stringency (FIG. 14A-C, G). A significant reduction in growth of BCL10^{E140X} but not BCL10^{WT} or BCL10^{R58Q} lymphomas (FIG. 14H-K) was observed, confirming their increased dependency on MALT1 activity and the potential for MALT1 inhibitors to be most useful for patients bearing such mutations.

### Discussion

Herein, it is shown that BCL10, one of the most frequently mutated genes in DLBCL, is a bona fide genetic driver of lymphomagenesis. Importantly, the structure-function studies reveal that these mutations occur in at least two biochemically distinct classes: missense mutations of the CARD domain and truncation mutations of the C-terminal tail. These classes of mutations seem to affect distinct aspects of BCL10 functionality and lead to biologically distinct outcomes as indicated by their differential downstream effects on MALT1 and NF-xB signaling as well as vulnerability to targeted therapies. Many of the BCL10 truncating mutations cluster between AA 135 to 174, and representatives of these mutations manifested the most powerful activation of NF-xB activity. BCL10 truncation mutants such as BCL10^{E140X} manifested a striking increase in its ability to polymerize into its filamentous form, accompanied by potent activation of MALT1 protease activity. This tendency to polymerize, indicated for example by its lower concentration threshold may help to explain the reduced CARD11 dependency of lymphoma cells expressing BCL10^{E140X}, and is consistent with previous studies showing that BCL10 CARD domain alone can undergo spontaneous polymerization in vitro. Although it is generally understood that CARD11 serves to nucleate BCL10 polymerization, it has been suggested that CARD11 association with BCL10 filaments is further stabilized by nascent helical BCL10 polymers. This may explain why increased association of BCL10 and CARD11 in lymphoma cells expressing BCL10^{E140X} was observed given its greater tendency to polymerize, while at the same time being consistent with its reduced requirement for CARD11 to induce filament formation and reduced biological dependency on CARD11 in BCL10^{E140X} expressing DLBCL cells.

Binding of the MALT1 death domain to BCL10 was unperturbed in filaments composed of BCL10^{E140X} which is not surprising since this molecular association is mediated through the BCL10 CARD domain and proximal regions that are not affected by truncation mutation. Yet Co-IP experiments paradoxically indicated reduced interaction between BCL10 and MALT1. This prompted us to examine other modes of BCL10-MALT1 association, leading to the identification of a novel direct interaction site between MALT1 Ig1-Ig2 region and BCL10 AAs 165 to 208, a region that is lost in a majority of truncation mutants except for a cluster deleting the extreme C-terminal Ser/Thr rich tail. Importantly, MALT1 impairs BCL10 polymerization through this interaction surface, thus constituting a novel CBM negative regulatory mechanism preventing spurious polymerization of BCL10. This in turn likely explains the dramatically increased filament formation by BCL10^{E140X} in vitro, and its greatly enhanced ability to recruit MALT1 into the polymerized CBM complex, given that loss of monomeric BCL10 and MALT1 binding would increase the pool of MALT1 to associate with filaments. These events occur due to the presence of BCL10^{E140X} thus appear to constitute a positive feedback loop that ultimately causes potent MALT1 protease activation and biological dependency on MALT1 catalytic function. The more distal set of C-terminal truncating mutations such as Q208X, L209X and L225X retain the MALT1 Ig1-Ig2 interacting region and hence would not be expected to escape from this MALT1 inhibitory binding. Accordingly, when expressed in cells, they did not induce greater NF-xB activity than WT BCL10, suggesting that there may be additional ways in which BCL10 function could be perturbed, perhaps due to specific loss of certain as of yet undiscovered post-translational modifications. Interestingly, L225X produces a truncated form of BCL10 similar to the MALT1 protease dependent cleavage form of BCL10 R228X observed in activated T-cells as well as in ABC-DLBCL cells with chronic active BCR signaling and showed similar functional effects to WT BCL10 overexpression in NF-xB reporter assays, perhaps due to retaining both intact MALT1 biding sites. Notably, the cleaved BCL10 R228X form was shown to mediate migratory function in T-cells. BCL10 truncating mutations, translocation and amplification were also shown to occur in marginal zone lymphomas including MALT lymphomas.

In contrast, missense mutations of the BCL10 CARD domain seem to have distinct functional effects. Many of the BCL10 residues affected by mutations (e.g., R58, K63) are localized within the core of BCL10 helical structures where they make important intrastrand (Type III) and interstrand (Type I) interactions that are involved in filament formation. Along these lines an R53Q mutation affecting type III interactions might be predicted to disrupt intrastrand interactions and caused a severe defect in BCL10 CARD domain polymerization. This was however proven to be incorrect, and the sole CARD domain hotspot mutant residue Q58 engages in a novel form of type III interaction resulting in apparent formation of a hydrogen bonded glutamine network. The consequence is a shift in BCL10 polymerization kinetics, favoring the polymerized state but without dramatically altering binding to CARD11 or MALT1 and yielding a more modest gain of function phenotype. Missense mutations such as K63Q might functionally resemble R58Q since they also locate at the central core of the BCL10 CARD filament, and have positively charged residues switched to glutamine to potentially enhance, rather than disrupt, interactions.

These biochemical features of the BCL10^{R58Q} mutant result in a hypomorphic phenotype compared to truncation mutant where they induced less potent MALT1 and NF-κB activation than BCL10^{E140X}. However, the results also suggest that BCL10^{R58Q} may engage in additional gain of function effects. This is suggested by the fact that cells expressing BCL10^{R58Q} seemed relatively resistant to loss of NF-κB activity upon exposure to MALT1 inhibitors as compared to that on BCL10-WT or BCL10^{E140X}, whereas in contrast, reduction in NF-κB activity in response to the upstream BTK inhibitors was similar between BCL10^{R58Q} and BCL10^{E140X}. These data prompt us to hypothesize that other functions may derive from the stability or bundling of R58Q filament. For example, more stable BCL10 filaments might enhance MALT1 recruitment and activation of TRAF6, which could partially support NF-κB independently from the MALT1 proteolytic function, or activate NF-κB through other alternative means such as linear ubiquitylation (LUBAC) associated mechanisms. Engagement of these or other MALT1 paracaspase independent biochemical effects would be consistent with BCL10^{R58Q} DLBCL cells retaining greater dependency on CARD11 and hence upstream signaling and responsiveness to BTKi.

Overall, our data spotlight the complexities involved in developing precision therapies for DLBCLs and other tumors. The identification of chronic active BCR signaling as a characteristic of ABC-DLBCLs has led to intense efforts to integrate BTKi into multi-modality regimens. However, to date, it is clear that many patients still do not benefit from the addition of such compounds. The data point to biochemical mechanisms that might help to explain this, as exemplified most clearly by the BTKi resistance conferred by BCL10 truncation mutants and this suggests that such patients should not be treated with BTKi containing regimens.

Instead, patients with BCL10 truncation mutations would likely best be served by incorporating MALT1 inhibitors. This concept is feasible since several MALT1 inhibitors are already in clinical trials. Although these findings could also be relevant to acquired BTK inhibitor resistance, as of yet BCL10 mutations have not been identified in this setting. Similar considerations may apply to other (but not all) mutations downstream of BTK, as exemplified by the case of CARD11 coiled-coil domain mutants, which induce resistance to BTKi but not MALT1i. However, the fact that BCL10 CARD domain mutations may still retain BTKi responsiveness further underlines the need for rigorous study of signaling pathway mutations such as these, and perhaps eventually the need for targeted sequencing studies to provide a precision therapy “map” of these tumors allowing selection (or even combination) of the targeted therapies (e.g., BTKi vs MALT1 i) appropriate to their specific signaling scenarios.

### Materials and Methods

### Cell Culture

Raji, HBL1, TMD8 and RIVA were cultured in RPMI supplemented with 10% FBS, 2 mM L-glutamine and 10 mM HEPES. OCI-Ly10 was cultured in Iscove's medium supplemented with 20% FBS, 2 mM L-glutamine. 293T was cultured in Dulbecco's Modified Eagle Medium with 10% FBS. All cell lines were authenticated by University of Arizona Genetic Core, grown in presence of 1% penicillin G and streptomycin and at 37° C. in a humidified atmosphere of 5% CO₂. HBL1 and RIVA was obtained from Jose A. Martinez-Climent (Universidad de Navarra, Pamplona, Spain); TMD8 was obtained from Louis M. Staudt (National Cancer Institute, Bethesda, Maryland, USA); OCI-Ly1 O cell lines were obtained from the Ontario Cancer Institute (OCI).

### Virus Production and Transduction

Lentiviruses were produced in 293T cells by co-transfecting shRNA (short hairpin sequences are: shNonTargeting: CAACAAGATGAAGAGCACCAA (SEQ ID NO:7); shCARD11#1: GGACGACAACTACAACTTAGC (SEQ ID NO:8); shCARD11#3: TGGTCAAGAAGCTGACGATTC (SEQ ID NO:9)) or overexpression vectors with packaging vectors psPax2 (Addgene #12260, RRID: Addgene_12260) and psMD2.g (Addgene #12259, RRID: Addgene_12259) at the 4:3:1 ratio in serum free media. The supernatant containing virus particles were harvested 48 h and 72 h after transfection, filtered through 0.45 μm filter and then concentrated with PEG-it according to manufacturer's instructions (LV825 Å-1, System Biosciences). Virus was resuspended with PBS containing 25 μM HEPES and added to cells for overnight infection. Cells were selected 24 h post transfection by adding puromycin (Sigma), blasticidin (Invivogen) or G418 (Life Technologies) for at least 48 h.

### Xenograft

All mice experiments were approved by Institutional Animal Care & Use Committee (IACUC) at Weill Cornell Medicine and were performed following the IACUC guidelines. Eight to ten weeks of female NOD.Cg-prkdc^{scid} Il2rg^{tm1Wj1}/SzJ (NSG, RRID: IMSR_JAX: 005557) mice were obtained from The Research Animal Resource Center (RARC) at Weill Cornell Medicine. 5×10⁶ HBL1 or 10⁷ million OCI-Ly and their derived engineered cells were resuspended with PBS/Matrigel (1:1) and subcutaneously injected to the right flank of mice. Treatments were started when tumor volume reached an average of 100 mm³. Ibrutinib was prepared in corn oil with 10% (v/v) DMSO or 0.5% methylcellulose in water and administrated p.o. with 25 or 37.5 mg/kg once per day. JNJ-67690246 was prepared in PEG400 with 10% (w/v) PVPVA64 and administrated p.o. with 100 mg/kg twice per day. Tumor volume was monitored 2-3 times/week with digital caliper and calculated using the following formula: smallest diameter²× largest diameter×0.5.

### Growth Inhibition Assay

DLBCL cell lines were cultured in exponential condition and the cell growth was determined by CellTiter Glo (Promega). 3000-5000 cells were seeded and cultured in each well of 384 well plate for 96 h and treated with compounds every 48 h. Luminescence was read at the endpoint with the Synergy NEO microplate reader (BioTek). The value of compound treated cells was normalized to their vehicle treated controls and then used to calculate IC50 in GraphPad Prism (RRID:SCR_002798).

### NF-κB Reporter Assay

For NF-κB reporter assay in 293T, the plasmids expressing different BCL10 mutations were transiently transfected into 293T cells using Lipofectamine (Invitrogen). Renilla luciferase plasmid was co-transfected as an internal control. 24 h after transfection, cells were collected and luciferase activities were measured in Synergy NEO microplate reader (BioTek) with the Dual Luciferase Reporter Assay System (Promega) according to the manufacturer's instructions and normalized to Renilla luciferase activity.

To generate stable NF-κB reporter cells, the lentivirus expressing 3×NF-κB response element followed by a luciferase firefly was made and infect the parental cells. Puromycin was then added for antibiotic selection 24 h after infection. Reporter cells were further validated by BTK inhibitor and MALT1 protease inhibitor treatment and PMA/1O stimulation. NF-κB reporter cells expressing BCL10 were generated by infecting reporter cells with different lentiviral BCL10 isoforms (co-expressing GFP), followed by sorting out GFP+ cells. Stable NF-κB reporter cells were harvested at the indicated conditions and lysed with 1× passive lysis buffer at room temperature for 20 min. The lysate was briefly centrifuged and the supernatant was collected for luciferase activity. All the assays were presented as mean±SEM of three independent experiments.

### MALT1 GloSensor Assay

The generation of Raji MALT1 GloSensor reporter cell has been described All other GloSensor reporter cells were generated by infecting parental cells with lentiviral MALT1-GloSensor (pLex306 backbone), followed by antibiotic (blasticidin) selection. All derived GloSensor cells were further validated by MALT1 protease inhibitor treatment and PMA/IO stimulation.

### Immunoprecipitation

10⁸ lymphoma cells were collected, washed with cold PBS and resuspended with lysis buffer (1% NP40, 10% glycerol, 150 mM NaCl, 20 mM Tris-HCL pH 7.5, and freshly added protease inhibitors). The lysates were centrifuged at 15,000 g, 4° C. for 15 min and the supernatant was then collected and incubated with 50 μL equilibrated anti-Flag magnetic beads (Sigma-Aldrich Cat #M8823, RRID:AB_2637089) at 4° C. for 3 h. The beads were washed 3 times with lysis buffer and followed by 3 times washing with the lysis buffer without NP40. SDS loading buffer without non-reducing reagent was added and boiled at 95° C. for 5 min. The elution was added for 0-mercaptoethanol (final 10%), and ready to run western blot after boiling at 95° C. for 5 min.

### Western Blotting

Whole cell lysates extracted with RIPA buffer or IP elution were separated by SDS-PAGE gels and followed by transferring to PVDF membranes. Membranes were incubated with indicated primary antibodies: anti-Flag (Sigma-Aldrich Cat #F3165, RRID:AB_259529), anti-MALT1 (Santa Cruz Biotechnology Cat #sc-46677, RRID:AB_627909), anti-CARD11 (Abcam Cat #ab113409, RRID:AB_10861854), anti-O-Actin (AC-15, Sigma-Aldrich), and then mouse/rabbit peroxidase-conjugated secondary antibodies (Cell Signaling Technology). Protein intensity was detected with enhanced chemiluminescence using ChemiDoc imaging system (Bio Rad).

### Driver Mutation Analysis

The Driver mutation analysis is performed using Fishhook (https.//github.com/mskilab/fishHook) on a total of 243 ABC-DLBCL cases from NCI cohort. Fishhook is a model built with mutational calls, a set of hypothesis intervals, eligible genomic ranges and a set of genomic covariates that identifies the depletion and enrichment of genomic interval statistically. The model used a gamma-Poisson regression to implement the maximum likelihood approximation with consideration of user assigned covariates and expected mutation density to the hypothesis. With this approach, the model helps us to identify enriched mutations with consideration like chromatin features, sequence context composition and gene expression.

Eligible region is defined using genecode v19 and fractional coverage of hg19 positions provided by Agilent exome coverage. We also fed the model covariates that defines B cell specific transcriptional states and chromatin state information for the model. The covariates of ABC specific transcriptional states are generated by number of the overlap between the TSS site of genes TPM >2 in the half the ABC-DLBCL cases from the same NCI cohort within 10 kb the eligible regions. The covariates of B-cell specific chromatin states are generated by number of the overlap between H3K27Ac Peaks that previously reported in the B cell within 100 kb of the eligible regions and the ATAC peaks of the B cells within 10 kb of the eligible. A total of 3 covariates was fed to the fishhook model. Here we noted genes of FDR<0.05 and BCL10 have an FDR of 1.4e⁻¹⁰. QQ-plot is plotted by pairing observed −log 10 transformed quantiles of observed P values (y-axis) with their corresponding −log 10 transformed quantiles from the uniform distribution (x-axis).

### Biochemical Evaluation of MALT1 Protease Activity

MALT1 protease activity was assessed in vitro using full-length MALT1 protein (Strep-MALT1 (1-824)-His) purified from baculovirus-infected insect cells. The tetrapeptide LRSR is coupled to 7-amino-4-methylcoumarin (AMC) and provides a quenched, fluorescent substrate for the MALT1 protease (SM Biochemicals). Cleavage of AMC from the arginine residue results in an increase in coumarin fluorescence measured at 460 nm (excitation 355 nm). Diluted compounds were pre-incubated with MALT1 enzyme for 50 minutes at room temperature (RT). Substrate was subsequently added and the reaction was then incubated for 4 h at RT, after which fluorescence was measured.

### Il-6/10 Secretion Assay Using DLBCL Cell Line

Secretion of the IL-6 and IL-10 cytokines by OCI-Ly3 ABC-DLBCL cells was measured using a Mesoscale assay (MSD). MALT1 inhibition results in a decrease of IL-6/10 secretion. OCI-Ly3 cells were treated with diluted compounds for 24 h at 37° C. and 5% CO2. After 24 h of incubation, 50 μL of the supernatant was transferred to an MSD plate (V-Plex Proinflammation Panel 1 [human] kit) and incubated for 2 h at RT followed by a 2 h incubation with IL-6/10 antibody solution. Plates were read on a SECTOR imager.

### Protein Expression and Purification

All constructs of BCL10, MALT1 are from human sequences. Full-length WT and mutant BCL10 constructs with N-terminal MBP tag were generated in vector pDB-His-MBP with a 3C protease site between MBP and BCL10. Full length His-tagged MALT1 cloned into pET29b was purchased from Addgene (RRID:Addgene_48968) and was expressed in E. coli.

All proteins were purified by either Ni-NTA resin (Qiagen) or Amylase resin followed by gel filtration chromatography (Superdex 200 10/300 GL, GE Healthcare). BCL10 FL and mutant filaments were purified by MBP affinity column in binding buffer containing 25 mM Tris at pH 7.5, 300 mM NaCl, 1 mM TCEP, followed by Superdex 200 gel filtration chromatography in buffer containing 20 mM Tris at pH 7.5, 150 mM NaCl and 1 mM TCEP, resulted in isolation of a monomeric fraction of BCL10. Then monomeric MBP-BCL10 was cleaved by 3C protease and incubated at RT for 2 hours in order to allow filaments formation. This step was followed by another Superdex 200 gel filtration chromatography and BCL10 filaments were isolated at the void peak for structure determination and thermostability assay. Isolation of BCL10 (1-140)-MALT1 complex was performed in a similar way in which BCL10 and MALT1 were purified separately. BCL10 pre-formed filaments after 3C cleavage were added mixed together with FL MALT1 to form a complex.

### Negative Stained Electron Microscopy

Copper grids coated with layers of plastic and thin carbon film were glow-charged before 5 μI of purified complexes were applied. Samples were left on the grids for 1 minute followed by negative staining with 1% uranyl formate for 30 seconds and air dried. In vitro BCL10^{WT} and mutants, BCL10/MALT1 and CBM were imaged with JEOL 1200EX or Tecnai G² Spirit BioTWIN at Harvard Medical School EM facility operating at 80 keV.

### Cryo-Electron Microscopy (Cryo-EM) Data Collection

Cryo grids for BCL10 R58Q and BCL10 E140X/MALT1 filaments were prepared by applying 3 μl of protein sample on a c-flat (1.2/1.3) 300 mesh grids. Grids were plunged by using vitrobot (FEI) at 4° c. with 3 sec blotting and force 4. For BCL10R58Q data collection, 3439 movies were collected at super resolution mode with using Arctica microscope at UMASS facility, operated at 200 kv facility with k2 camera. The movies were collected automatically using SerialEM data collection at a nominal magnification of 36,000 and a pixel size of 0.435 Å, with a total dose of 38 e/Å2 which was fractionated into 40 movie frames, with defocus range of −1-2.5 μm. For BCL10 E140X-MALT1 collection, 700 movies were collected at super resolution mode with using 300 kV FEI Titan Krios microscope equipped with FEI Falcon II detector at PNCC cryo-EM facility, operated at 300 kv with Falcon3 camera. The movies were collected automatically using SerialEM data collection at a nominal magnification of 47,000 and a pixel size of 0.4 Å, with a total dose of 55 e/Å2 which was fractionated into 40 movie frames.

### Cryo Electron Data Processing

For helical reconstruction of BCL10 R58Q and BCL10 E140X/MALT1, Motioncor2 was used for drift correction and Micrographs were CTF corrected by using CTFFIND4. Data was processed with using Relion (3.1). The resolutions of the reconstruction were determined by FSC to 4.6 Å and 4.3 Å, respectively. Model building was performed in program Coot36. Refinement was performed against the using Phenix refine50. Structural presentations were generated using Pymol (Delano Scientific) and Chimera.

### Confocal Imaging

Time lapsed movies of full length labeled Alexa488-BCL10 FL, BCL10 R58Q and BCL10 E140X were recorded with using Nikon spinning disk confocal microscope at Harvard Micron facility for periods of 30 min-1 hr with 1 minute interval, with using ×100 objective. 3C was added at a sub-molar ratio to allow MBP cleavage to occur within 2-3 minutes in order to provide ample time for setting up the microscope and starting the recording.

For concentration determination, labeled Alexa488-BCL10 FL, R58Q and E140X filaments were formed at increasing concentrations ranging between 0.1 M-1 M. 4 h after cleavage with 3C and incubation at RT, samples were placed on a 35 mm bottom glass dish and imaged with using spinning disk confocal microscope with using ×100 objective.

### Fluorescence Quenching Assay

Purified full length MBP-BCL10, BCL10 R58Q and BCL10 E140X were mixed with 5-fold molar excess of Alexa-488-C5-maleimide (Invitrogen) and incubated at 4° C. temperature for O/N. Gel filtration chromatography (Superdex 200, GE Healthcare) was used to remove free dyes. Fluorescence polarization assay was performed at 18° C. in buffer containing 20 mM Tris at pH 7.5, 150 mM NaCl, and 0.5 mM TCEP and in 20 μI volume. 3 μM of labeled MBP-BCL10 were cleaved with 3C in the presence of increasing amount of MALT1. The fluorescence quenching was measured right after 3C addition for 2 h by using NEO plate reader (Biotek) using excitation/emission wavelengths of 495 nm/519 nm.

### Protein Stability

Purified BCL10 FL, R58Q and E140X filaments purified from the void peak of Superdex200 were mixed with 1-fold protein thermal shift dye (Thermo Fisher Scientific). Thermal scanning (25 to 95° C. at 1° C./min) was performed and melting curves were recorded on a StepOne RT-PCR machine. Data analysis was done by Protein Thermal Shift™ Software (Thermo Fisher Scientific).

### IF

HBL1 cells cultured in fresh media were mixed in 1:1 ratio with cytospin. Cells were spined at 800×g for 5 min. Cell pellets were resuspended with cytospin and plated on CELLview 4-compartment dishes (Greiner Bio-One). Cells were left at RT o/n and were fixed with 100% cold methanol for 5 minutes at −20° C., followed by cell permeabilization with 0.1% Triton X-100 in PBS-Tween (PSST) for 10 minutes. Cells were incubated with blocking buffer containing 3% BSA for 3 h, in order to minimize non-specific binding. After blocking. Cells were incubated overnight at 4° C. with FLAG primary antibody (Sigma-Aldrich Cat #F1804, RRID:AB_262044). After incubation, cells were washed with PBST 3 times and incubated with AlexaFluor488-conjugated anti-mouse IgG (Abcam Cat #ab150113, RRID:AB_2576208) 5 for 1 h at room temperature. After incubation, cells were washed with PBS and then stained with Hoechst for 10 minutes (1:500, Immunochemistry Technologies, Cat #639). Cells were imaged using spinning disk confocal microscope with using ×100 objective.

IHC staining of p65

Formalin-fixed paraffin-embedded (FFPE) tissue sections of 4 μm thickness were cut from a tissue microarray composed of duplicate 0.6 mm cores from 298 cases of de nova DLBCL. Slides were processed using standard immunohistochemistry protocols and stained with an antibody against NF-xB p65 (Cell Signaling Technology Cat #8242, RRID:AB_10859369, 1:500 dilution). Appropriate staining was verified in sections of benign tonsil, heart and liver. Stained slides were assessed by an expert hematopathologist for nuclear expression of p65 in DLBCL tumor cells, scored as a percentage of tumor cell nuclei.

### REFERENCES

Aalipour & Advani, Ther. Adv. Hematol., 5:121 (2014).
Alizadeh et al., Nature, 403:503 (2000).
Caeser et al., JCO Precis. Oncol., 5:145 (2021).
Campanello et al., PLoS Comput. Biol., 17:e1007986 (2021).
Chan et al., Mol. Cell. Biol., 33:429 (2013).
Chapuy et al., Nat. Med., 24:679 (2018).
Cheng et al., Cell. Immunol., 355:104158 (2020).
David et al., Proc. Natl. Acad. Sci. U.S.A., 115:1499 (2018).
Davis et al., Nature, 463:88 (2010).
Du et al., Blood 95:3885 (2000).
Dubois et al., Blood, 123:2199 (2014).
Fontan et al., J. Clin. Invest., 128:4397 (2018).
Hailfinger et al., Proc. Natl. Acad. Sci. U.S.A., 106:19946 (2009).
Hendriks et al., Nat. Rev. Cancer, 14:219 (2014).
Imielinski et al., Cell, 168:460 (2017).
Johansson et al., Oncotarget, 2:62627 (2016).
Juilland & Thome, Curr. Opin. Hematol., 23:402 (2016).
Karube et al., Leukemia, 32:675 (2018).
Koseki et al., J. Biol. Chem., 274:9955 (1999).
Lacy et al., Blood, 135:1759 (2020).
Lamason et al., Biochemistry, 49:8240 (2010).
Lenz et al., Science, 319:1676 (2008).
Lu et al., in ACS Spring 2021 (12:45 pm-01:25 pm USA/Canada-Eastern—Apr. 9, 2021).
Lucas et al., J. Biol. Chem., 276:19012 (2001).
McCully & Pomerantz, Mol. Cell. Biol., 28:5668 (2008).
Meininger & Krappmann, Biol. Chem., 397:1315 (2016).
Morin et al., Blood, 122:1256 (2013).
Netea et al., Nat. Immunol., 13:535 (2012).
Ngo et al., Nature, 470:115 (2011).
Paul et al., Immunity, 36:947 (2012).
Pettersen et al., J. Comput. Chem., 25:1605 (2004).
Philippar et al., Cancer Res., 80:_(2020).
Qiao et al., Mol. Cell. 51:766 (2013).
Quancard et al., Nat. Chem. Biol., 15:304 (2019)
Rawling & Pyle, Curr. Opin. Struct. Biol., 25:25 (2014).
Rebeaud et al., Nat. Immunol., 9:272 (2008).
Reddy et al., Cell, 171:481 (2017).
Rossman et al., Mol. Biol. Cell., 17:2166 (2006).
Sanchez-Izquierdo et al., Blood, 101:4539 (2003).
Schlauderer et al., Nat. Commun., 9:4041 (2018).
Schmitz et al., N. Engl. J. Med., 378:1396 (2018).
Shinohara et al., J. Exp. Med., 204:3285 (2007).
Song & Lee, Immunol. Rev., 250:216 (2012).
Stinson et al., Cell. Immunol., 353:104129 (2020).
Thome et al., Cold Spring Harb. Perspect. Biol., 2:a003004 (2010).
Thys et al., Front Oncol., 8:498 (2018).
Traver et al., Methods Mol. Biol., 1584:101 (2017).
Turvey et al., J. Allergy Clin. Immunol., 134:276 (2014).
Vela et al., Hematol. Oncol., 38:284 (2020).
Vicente-Duenas et al., Proc. Natl. Acad. Sci. U.S.A., 109:10534 (2012).
Willis et al., Cell, 96:35 (1999).
Wilson et al., Nat. Med., 21:922 (2015).
Woyach et al., N. Engl. J. Med., 370:2286 (2014).
Wright et al., Cancer Cell., 37:551 (2020).
Yan et al., J. Biol. Chem., 274:10287 (1999).
Yang et al., Cancer Discov., 4:480 (2014).
Young et al., Semin. Hematol., 52:77 (2015).
Zhou et al., Nature, 427:167 (2004).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following Statements summarize aspects and features of the invention.

### Statements:

1. A method comprising administering at least one MALT1 inhibitor to a subject having one or more genomic mutations in a coding region of at least one BCL10 allele.
2. The method of statement 1, wherein the one or more genomic mutations comprise:
   a. a mutation in a BCL10 CARD domain;
   b. a BCL10 C-terminal deletion; or
   c. a combination thereof.
3. The method of statement 1 or 2, wherein the subject has a genomic mutation in at least one BCL10 coding region comprising 1-10 amino acids encompassing position 58 of SEQ ID NO:1.
4. The method of any of statements 1-3, wherein the subject has a genomic mutation in at least one BCL10 coding region at an amino acid position equivalent to position 58 of SEQ ID NO:1.
5. The method of any of statements 1-4, wherein the subject has a C-terminal deletion in at least one BCL10 allele at positions equivalent to amino acid positions 135 to 174 of SEQ ID NO:1.
6. The method of any of statements 1-6, wherein one or more of the mutations in at least one BCL10 allele is a missense mutation within a position corresponding to position 58 of SEQ ID NO:1 or within a position within 1-10 amino acids of position 58 of SEQ ID NO:1.
7. The method of any of statements 1-7, wherein one or more of the mutations in at least one BCL10 allele is a deletion of at least two, at least three, at least four, at least 5, at least 7, at least 10, at least 15, at least 20, at least 25, or at least 30 nucleotides within positions equivalent to amino acid positions 135 to 174 of SEQ ID NO:1.
8. The method of any one of statements 1-7, wherein the subject is not administered a BTK inhibitor.
9. The method of any one of statements 1-7, wherein the subject was previously administered a BTK inhibitor.
10. The method of any one of statements 1-7, wherein the subject was previously not administered a BTK inhibitor.
11. The method of any of statements 8-10, wherein the BTK inhibitor is Ibrutinib, Zanubrutinib, Acalabrutinib, CGI 1746, LCB 03-0110, LFM-A13, PCI 29732, PF 06465469, (−)-Terreic acid, DD 03-171, or a combination thereof.
12. The method of any one of statements 1-7, wherein the subject is administered at least one MALT1 inhibitor for at least 25 days, at least 30 days, at least 35 days, at least 40 days, at least 50 days, at least 60 days, at least 90 days, or at least 180 days.
13. The method of any of statements 1-12, wherein at least one MALT1 inhibitor is MLT-748, JNJ-67690246, MI-2, Mepazine, MLT-943, JNJ-67856633, MLT-985, (R)-MAL T1-IN-7, MLT-231, Z-VRPR-FMK (TFA), MLT-747, (S)-MALT1-IN-5, (R)-MALT1-IN-3, MALT1-IN-7, or any combination thereof.
14. The method of any one of statements 1-13, wherein the subject is suspected of having lymphoma, or may develop lymphoma.
15. The method of any one of statements 1-14, wherein tumor weight or tumor volume in the subject is reduced by at least 10%, or 20% or 30%, or 40%, or 50%, or 60%, or 75%, or 100% compared to an untreated control.
16. The method of any one of statements 1-15, wherein tumor weight or tumor volume in the subject is reduced by at least 1.2-fold, or 1.5-fold, or 2-fold, or 3-fold, or 5-fold, or 7-fold, or 10-fold compared to an untreated control.
17. A method comprising:
   a. testing at least one sample from at least one subject to identify one or more subjects that have one or more genomic mutations in a coding region of at least one BCL10 allele; and
   b. administering at least one MALT1 inhibitor to any subjects having one or more of the genomic mutations in a coding region of at least one of the BCL10 alleles.
18. The method of statement 17, wherein the one or more genomic mutations comprise:
   a. a mutation in a BCL10 CARD domain;
   b. a BCL10 C-terminal deletion; or
   c. a combination thereof.
19. The method of statement 17 or 18, wherein the one or more genomic mutations is in at least one BCL10 coding region comprising 1-10 amino acids encompassing position 58 of SEQ ID NO:1.
20. The method of any of statements 17-19, wherein the subject has a genomic mutation in at least one BCL10 coding region at an amino acid position equivalent to position 58 of SEQ ID NO:1.
21. The method of any of statements 17-20, wherein one or more of the mutations in at least one BCL10 allele is a missense mutation within a position corresponding to position 58 of SEQ ID NO:1 or within a position within 1-10 amino acids of position 58 of SEQ ID NO:1.
22. The method of any of statements 17-21, wherein one or more of the mutations in at least one BCL10 allele is a deletion of at least two, at least three, at least four, at least 5, at least 7, at least 10, at least 15, at least 20, at least 25, or at least 30 nucleotides within positions equivalent to amino acid positions 135 to 174 of SEQ ID NO:1.
23. The method of any of statements 17-22, wherein the subject has a C-terminal deletion in at least one BCL10 allele at positions equivalent to amino acid positions 135 to 174 of SEQ ID NO:1.
24. The method of any one of statements 17-23, wherein the subject is not administered a BTK inhibitor.
25. The method of any one of statements 17-24, wherein the subject was previously administered a BTK inhibitor.
26. The method of any one of statements 17-25, wherein the subject was previously not administered a BTK inhibitor.
27. The method of any of statements 23-26, wherein the BTK inhibitor is Ibrutinib, Zanubrutinib, Acalabrutinib, CGI 1746, LCB 03-0110, LFM-A13, PCI 29732, PF 06465469, (−)-Terreic acid, DD 03-171, or a combination thereof.
28. The method of any one of statements 17-27, wherein the subject is administered at least one MALT1 inhibitor for at least 25 days, at least 30 days, at least 35 days, at least 40 days, at least 50 days, at least 60 days, at least 90 days, or at least 180 days.
29. The method of any of statements 17-28, wherein at least one MALT1 inhibitor is MLT-748, JNJ-67690246, MI-2, Mepazine, MLT-943, JNJ-67856633, MLT-985, (R)-MALT1-IN-7, MLT-231, Z-VRPR-FMK (TFA), MLT-747, (S)-MALT1-IN-5, (R)-MALT1-IN-3, MALT1-IN-7, or any combination thereof
30. The method of any one of statements 17-29, wherein tumor weight or tumor volume in the subject is reduced by at least 10%, or 20% or 30%, or 40%, or 50%, or 60%, or 75%, or 100% compared to an untreated control.
31. The method of any one of statements 17-30, wherein tumor weight or tumor volume in the subject is reduced by at least 1.2-fold, or 1.5-fold, or 2-fold, or 3-fold, or 5-fold, or 7-fold, or 10-fold compared to an untreated control.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed is:

1. A method comprising administering at least one MALT1 inhibitor to a subject having one or more genomic mutations in a coding region of at least one BCL10 allele.

2. The method of claim 1, wherein the one or more genomic mutations comprise:
a. a mutation in a BCL10 CARD domain;
b. a BCL10 C-terminal deletion; or
c. a combination thereof.

3. The method of claim 1, wherein the subject has a genomic mutation in at least one BCL10 coding region comprising 1-10 amino acids encompassing position 58 of SEQ ID NO:1.

4. The method of claim 1, wherein the subject has a genomic mutation in at least one BCL10 coding region at an amino acid position equivalent to position 58 of SEQ ID NO:1.

5. The method of claim 1, wherein the subject has a C-terminal deletion in at least one BCL10 allele at positions equivalent to amino acid positions 135 to 174 of SEQ ID NO:1.

6. The method of claim 1, wherein one or more of the mutations in at least one BCL10 allele is a missense mutation within a position corresponding to position 58 of SEQ ID NO:1 or within a position within 1-10 amino acids of position 58 of SEQ ID NO:1.

7. The method of claim 1, wherein one or more of the mutations in at least one BCL10 allele is a deletion of at least two, at least three, at least four, at least 5, at least 7, at least 10, at least 15, at least 20, at least 25, or at least 30 nucleotides within positions equivalent to amino acid positions 135 to 174 of SEQ ID NO:1.

8. The method of claim 1, wherein the subject is not administered a BTK inhibitor.

9. The method of claim 1, wherein the subject was previously administered a BTK inhibitor.

10. The method of claim 1, wherein the subject was previously not administered a BTK inhibitor.

11. The method of claim 9, wherein the BTK inhibitor is lbrutinib, Zanubrutinib, Acalabrutinib, CGI 1746, LCB 03-0110, LFM-A13, PCI 29732, PF 06465469, (−)-Terreic acid, DD 03-171, or a combination thereof.

12. The method of claim 1, wherein the subject is administered at least one MALT1 inhibitor for at least 25 days, at least 30 days, at least 35 days, at least 40 days, at least 50 days, at least 60 days, at least 90 days, or at least 180 days.

13. The method of claim 1, wherein at least one MALT1 inhibitor is MLT-748, JNJ-67690246, MI-2, Mepazine, MLT-943, JNJ-67856633, MLT-985, (R)-MALT1-IN-7, MLT-231, Z-VRPR-FMK (TFA), MLT-747, (S)-MALT1-IN-5, (R)-MALT1-IN-3, MALT1-IN-7, or any combination thereof.

14. The method of claim 1, wherein the subject is suspected of having lymphoma, or may develop lymphoma.

15. The method of claim 1, wherein tumor weight or tumor volume in the subject is reduced by at least 10%, or 20% or 30%, or 40%, or 50%, or 60%, or 75%, or 100% compared to an untreated control.

16. The method of claim 1, wherein tumor weight or tumor volume in the subject is reduced by at least 1.2-fold, or 1.5-fold, or 2-fold, or 3-fold, or 5-fold, or 7-fold, or 10-fold compared to an untreated control.

17. A method comprising:
testing at least one sample from at least one subject to identify one or more subjects that have one or more genomic mutations in a coding region of at least one BCL10 allele; and
administering at least one MALT1 inhibitor to any subjects having one or more of the genomic mutations in a coding region of at least one of the BCL10 alleles.

18. The method of claim 17, wherein the one or more genomic mutations comprise:
d. a mutation in a BCL10 CARD domain;
e. a BCL10 C-terminal deletion; or
f. a combination thereof.

19. The method of claim 17, wherein the one or more genomic mutations is in at least one BCL10 coding region comprising 1-10 amino acids encompassing position 58 of SEQ ID NO:1, wherein the subject has a genomic mutation in at least one BCL10 coding region at an amino acid position equivalent to position 58 of SEQ ID NO:1, wherein one or more of the mutations in at least one BCL10 allele is a missense mutation within a position corresponding to position 58 of SEQ ID NO:1 or within a position within 1-10 amino acids of position 58 of SEQ ID NO:1, wherein one or more of the mutations in at least one BCL10 allele is a deletion of at least two, at least three, at least four, at least 5, at least 7, at least 10, at least 15, at least 20, at least 25, or at least 30 nucleotides within positions equivalent to amino acid positions 135 to 174 of SEQ ID NO:1, or wherein the subject has a C-terminal deletion in at least one BCL10 allele at positions equivalent to amino acid positions 135 to 174 of SEQ ID NO:1.

20. The method of claim 17, wherein at least one MALT1 inhibitor is MLT-748, JNJ-67690246, MI-2, Mepazine, MLT-943, JNJ-67856633, MLT-985, (R)-MALT1-IN-7, MLT-231, Z-VRPR-FMK (TFA), MLT-747, (S)-MALT1-IN-5, (R)-MALT1-IN-3, MALT1-IN-7, or any combination thereof.
